(19) United States
(12) Patent Application Publication
(10) Pub. No.: US 2019/0241548 A1
(43) Pub. Date: Aug. 8, 2019

(54) COMPOUND FOR ORGANIC ELECTRONIC ELEMENT, ORGANIC ELECTRONIC ELEMENT USING SAME, AND ELECTRONIC DEVICE COMPRISING SAME

(71) Applicant: DUK SAN NEOLUX CO., LTD., Cheonan-si, Chungcheongnam-do (KR)

(72) Inventors: Hyoung Keun PARK, Chuncheon-si, Gangwon-do (KR); Sun Hee LEE, Hwaseong-si, Gyeonggi-do (KR); Yun Suk LEE, Seongnam-si, Gyeonggi-do (KR); Ki Ho SO, Cheonan-si, Chungcheongnam-do (KR); Jong Gwang PARK, Ulsan (KR); Yeon Seok JEONG, Gangwon-do (KR); Soung Yun MUN, Cheonan-si, Chungcheongnam-do (KR); Jae Taek KWON, Cheonan-si, Chungcheongnam-do (KR)

(73) Assignee: DUK SAN NEOLUX CO., LTD., Cheonan-si, Chungcheongnam-do (KR)

(21) Appl. No.: 15/779,122

(22) PCT Filed: Nov. 10, 2016
(86) PCT No.: PCT/KR2016/012921

§ 371 Date: Oct. 29, 2018

(30) Foreign Application Priority Data
Nov. 26, 2015 (KR) 10-2015-0166001
Nov. 9, 2016 (KR) 10-2016-0148884

(51) Int. Cl. C07D 409/04; C07D 405/04; C07D 405/14; C07D 409/14; H01L 51/00

(52) U.S. Cl. CPC C07D 409/04; C07D 405/04; C07D 405/14; C07D 409/14; H01L 51/0058; H01L 51/5012; H01L 51/006; H01L 51/0061; H01L 51/0072; H01L 51/0073; H01L 51/0074; H01L 51/0067

(57) ABSTRACT

The present invention provides a novel compound capable of improving the luminous efficiency, stability and life of an element, an organic electronic element using the same, and an electronic device comprising same.

### BACKGROUND

### Technical Field

The present invention relates to compound for organic electronic element, organic electronic element using the same, and an electronic device thereof.

### Background Art

In general, organic light emitting phenomenon refers to a phenomenon that converts electric energy into light energy by using an organic material. An organic electric element using an organic light emitting phenomenon usually has a structure including an anode, a cathode, and an organic material layer interposed therebetween. Here, in order to increase the efficiency and stability of the organic electronic element, the organic material layer is often composed of a multi-layered structure composed of different materials, and for example, may include a hole injection layer, a hole transport layer, an emitting layer, an electron transport layer, an electron injection layer and the like.

A material used as an organic material layer in an organic electric element may be classified into a light emitting material and a charge transport material, such as a hole injection material, a hole transport material, an electron transport material, an electron injection material and the like depending on its function.

In the organic electroluminescent device, the most problematic is the lifetime and the efficiency. As the display becomes large, such efficiency and the lifetime problem must be solved.

Efficiency, life span, driving voltage and the like are related to each other. As the efficiency is increased, the driving voltage is relatively decreased, and as the driving voltage drops, the crystallization of the organic material due to joule heating generated during driving is reduced, and as a result, the life span tends to increase.

However, simply improving the organic material layer cannot maximize the efficiency. This is because, when the optimal combination of the energy level and T1 value between each organic material layer and the intrinsic properties (mobility, interface characteristics, etc.) of the material are achieved, long life and high efficiency can be achieved at the same time.

Further, recently, in organic electroluminescent devices, in order to solve the emission problem in the a hole transport layer, an electron blocking layer must be present between the hole transport layer and an emitting layer, and it is necessary to develop an electron blocking layer commonly used for each of the emitting layers (R, G, B)

In general, electrons are transferred from the electron transport layer to the emitting layer, and holes are transferred from the hole transport layer to the emitting layer to generate excitons by recombination.

However, the material used for the hole transport layer has a low HOMO value and therefore has mostly low T1 value. As a result, the exciton generated in the emitting layer is transferred to the hole transport layer, resulting in charge unbalance in the emitting layer, and light is emitted at the interface of the hole transport layer.

When light is emitted from the interface of the hole transport layer, color purity and efficiency of the organic electronic device are lowered and the lifetime is shortened. Therefore, it is urgently required to develop an emitting auxiliary layer having a high T1 value and having a HOMO level between the HOMO energy level of the hole transport layer and the HOMO energy level of the emitting layer.

In addition, it is necessary to develop a hole injection layer material that retards penetration/diffusion of metal oxides from an anode electrode (ITO) into an organic material layer, which is one cause for the shortened life span of an organic electric element, and has stability against Joule heating generated during the operation of an organic electric element, that is, a high glass transition temperature. Also, it has been reported that a low glass transition temperature of a hole transport layer material has a great effect on the life span of an organic electric element because the uniformity of a thin film surface collapses during the operation of the element. In general, deposition is a main method of forming an OLED, and thus it is necessary to develop a material that is durable to such a deposition method, that is, a highly heat-resistant material.

That is, in order to sufficiently exhibit the excellent characteristics of the organic electric element, a material for forming an organic material layer in an element such as a hole injection material, a hole transport material, a light emitting material, an electron transport material, an electron injection material, an electron blocking layer material should be supported by stable and efficient materials. However, such a stable and efficient organic material layer material for an organic electric element has not been sufficiently developed yet. Therefore, development of new materials is continuously required, and development of materials for the electron blocking layer and the hole transport layer is urgently required.

A prior art reference is KR1020130076842A.

### DETAILED DESCRIPTION OF THE INVENTION

### SUMMARY

In order to solve the problems of the background art described above, an embodiment of the present invention has revealed a compound having a novel structure, and it has also been found that when the compound is applied to an organic electronic device, the luminous efficiency, stability and lifetime of the device can be greatly improved.

An object of the present invention is to provide a novel compound, an organic electric element using the same and an electronic device thereof.

### Technical Solution

The present invention provides a compound represented by Formula(1) below and a composition for a hole transport layer or an emitting-auxiliary layer comprising the same and an organic electric element characterized in having the same.

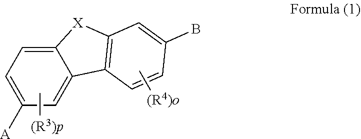

### Effects of the Invention

By using the compound according to the present invention, it is possible to achieve a high luminous efficiency, a low driving voltage, and a high heat resistance of the element, and can greatly improve the color purity and lifetime of the element.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is an illustration of an organic electric element according to the present invention.

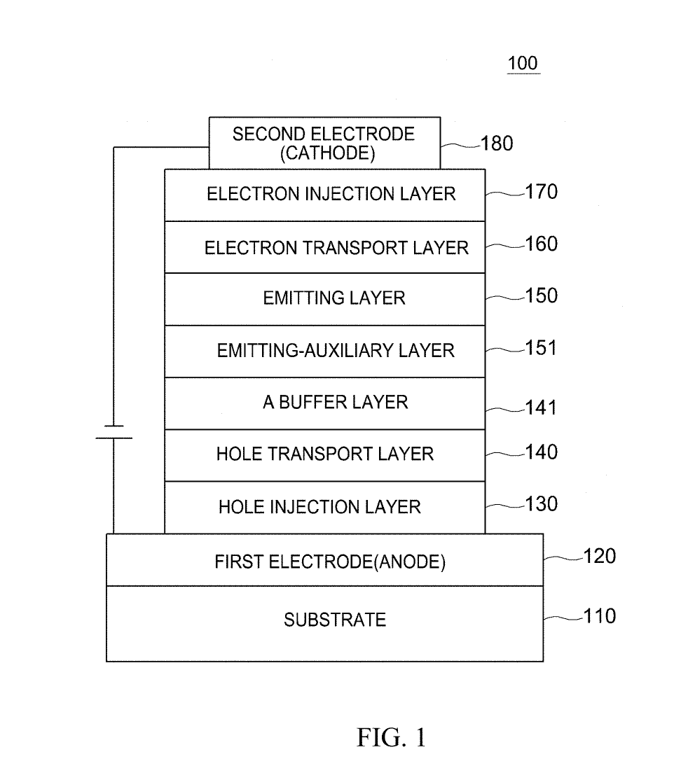

| | |
|---|---|
| 100: organic electric element, | 110: substrate |
| 120: the first electrode(anode), | 130: the hole injection layer |
| 140: the hole transport layer, | 141: a buffer layer |
| 150: the emitting layer, | 151: the emitting auxiliary layer |
| 160: the electron transport layer, | 170: the electron injection layer |
| 180: the second electrode(cathode) | |

FIG. 2 is an illustration of electronic cloud degree of HOMO and LUMO of Comparative compound and present compound.

### DETAILED DESCRIPTION

Hereinafter, some embodiments of the present invention will be described in detail. Further, in the following description of the present invention, a detailed description of known functions and configurations incorporated herein will be omitted when it may make the subject matter of the present invention rather unclear.

In addition, terms, such as first, second, A, B, (a), (b) or the like may be used herein when describing components of the present invention. Each of these terminologies is not used to define an essence, order or sequence of a corresponding component but used merely to distinguish the corresponding component from other component(s). It should be noted that if a component is described as being “connected”, “coupled”, or “connected” to another component, the component may be directly connected or connected to the other component, but another component may be “connected “, ” coupled” or “connected” between each component.

As used in the specification and the accompanying claims, unless otherwise stated, the following is the meaning of the term as follows.

Unless otherwise stated, the term “halo” or “halogen”, as used herein, comprises fluorine, bromine, chlorine, or iodine.

Unless otherwise stated, the term “alkyl” or “alkyl group”, as used herein, has a single bond of 1 to 60 carbon atoms, and means saturated aliphatic functional radicals including a linear alkyl group, a branched chain alkyl group, a cycloalkyl group (alicyclic), an cycloalkyl group substituted with a alkyl or an alkyl group substituted with a cycloalkyl.

Unless otherwise stated, the term “haloalkyl” or “halogen alkyl”, as used herein, comprises an alkyl group substituted with a halogen.

Unless otherwise stated, the term “heteroalkyl”, as used herein, means alkyl substituted one or more of carbon atoms consisting of an alkyl with hetero atom.

Unless otherwise stated, the term “alkenyl” or “alkynyl”, as used herein, has double or triple bonds of 2 to 60 carbon atoms, but is not limited thereto, and comprises a linear or a branched chain group.

Unless otherwise stated, the term “cycloalkyl”, as used herein, means alkyl forming a ring having 3 to 60 carbon atoms, but is not limited thereto.

Unless otherwise stated, the term “alkoxyl group”, “alkoxy group” or “alkyloxy group”, as used herein, means an oxygen radical attached to an alkyl group, but is not limited thereto, and has 1 to 60 carbon atoms.

Unless otherwise stated, the term “alkenoxyl group”, “alkenoxy group”, “alkenyloxy group” or “alkenyloxy group”, as used herein, means an oxygen radical attached to an alkenyl group, but is not limited thereto, and has 2 to 60 carbon atoms.

Unless otherwise stated, the term “aryloxyl group” or “aryloxy group”, as used herein, means an oxygen radical attached to an aryl group, but is not limited thereto, and has 6 to 60 carbon atoms.

Unless otherwise stated, the term “aryl group” or “arylene group”, as used herein, has 6 to 60 carbon atoms, but is not limited thereto. Herein, the aryl group or arylene group means a monocyclic and polycyclic aromatic group, and includes an aromatic ring formed by neighboring substituents participating in a bond or a reaction. Examples of “aryl group” may comprise a phenyl group, a biphenyl group, a fluorene group, or a spirofluorene group.

The prefix “aryl” or “ar” means a radical substituted with an aryl group. For example, an arylalkyl may be an alkyl substituted with an aryl, and an arylalenyl may be an alkenyl substituted with aryl, and a radical substituted with an aryl has a number of carbon atoms as defined herein.

Also, when prefixes are named subsequently, it means that substituents are listed in the order described first. For example, an arylalkoxy means an alkoxy substituted with an aryl, an alkoxylcarbonyl means a carbonyl substituted with an alkoxyl, and an arylcarbonylalkenyl also means an alkenyl substituted with an arylcarbonyl, wherein the arylcarbonyl may be a carbonyl substituted with an aryl.

Unless otherwise stated, the term “heteroalkyl”, as used herein, means alkyl containing one or more of hetero atoms. Unless otherwise stated, the term “heteroaryl group” or “heteroarylene group”, as used herein, means a C₂ to C₆₀ aryl containing one or more of hetero atoms or arylene group, but is not limited thereto, and comprises at least one of monocyclic and polycyclic rings, and may also be formed in conjunction with an adjacent group.

Unless otherwise stated, the term “heterocyclic group”, as used herein, contains one or more heteroatoms, but is not limited thereto, has 2 to 60 carbon atoms, comprises any one of monocyclic and Polycyclic rings, and may comprise heteroaliphadic ring and/or heteroaromatic ring. Also, the heterocyclic group may also be formed in conjunction with an adjacent group.

Unless otherwise stated, the term “heteroatom”, as used herein, represents at least one of N, O, S, P, or Si.

Also, the term “heterocyclic group” may comprise a ring containing SO₂ instead of carbon consisting of cycle. For example, “heterocyclic group” comprises compound below.

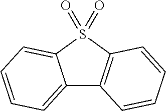 [SMILES: O=S1(=O)c2ccccc2-c2ccccc21]

Unless otherwise stated, the term “aliphatic”, as used herein, means an aliphatic hydrocarbon having 1 to 60 carbon atoms, and the term “aliphatic ring”, as used herein, means an aliphatic hydrocarbon ring having 3 to 60 carbon atoms.

Unless otherwise stated, the term “ring”, as used herein, means an aliphatic ring having 3 to 60 carbon atoms, or an aromatic ring having 6 to 60 carbon atoms, or a hetero ring having 2 to 60 carbon atoms, or a fused ring formed by the combination of them, and comprises a saturated or unsaturated ring.

Other hetero compounds or hetero radicals other than the above-mentioned hetero compounds contain, but are not limited thereto, one or more heteroatoms.

Unless otherwise stated, the term “carbonyl”, as used herein, is represented by —COR′, wherein R′ may be hydrogen, an alkyl having 1 to 20 carbon atoms, an aryl having 6 to 30 carbon atoms, a cycloalkyl having 3 to 30 carbon atoms, an alkenyl having 2 to 20 carbon atoms, an alkynyl having 2 to 20 carbon atoms, or the combination of these.

Unless otherwise stated, the term “ether”, as used herein, is represented by —R—O—R′, wherein R or R′ may be independently hydrogen, an alkyl having 1 to 20 carbon atoms, an aryl having 6 to 30 carbon atoms, a cycloalkyl having 3 to 30 carbon atoms, an alkenyl having 2 to 20 carbon atoms, an alkynyl having 2 to 20 carbon atoms, or the combination of these.

Unless otherwise stated, the term “substituted or unsubstituted”, as used herein, means that substitution is substituted by at least one substituent selected from the group consisting of, but is not limited thereto, deuterium, halogen, an amino group, a nitrile group, a nitro group, a C₁-C₂₀ alkyl group, a C₁-C₂₀ alkoxyl group, a C₁-C₂₀ alkylamine group, a C₁-C₂₀ alkylthiopen group, a C₆-C₂₀ arylthiopen group, a C₂-C₂₀ alkenyl group, a C₂-C₂₀ alkynyl group, a C₃-C₂₀ cycloalkyl group, a C₆-C₂₀ aryl group, a C₆-C₂₀ aryl group substituted by deuterium, a C₈-C₂₀ arylalkenyl group, a silane group, a boron group, a germanium group, and a C₂-C₂₀ heterocyclic group.

Unless otherwise expressly stated, the Formula used in the present invention, as used herein, is applied in the same manner as the substituent definition according to the definition of the exponent of the following Formula.

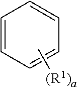

wherein, when a is an integer of zero, the substituent R¹ is absent, when a is an integer of 1, the sole substituent R¹ is linked to any one of the carbon constituting the benzene ring, when a is an integer of 2 or 3, each substituent R¹s may be the same and different, when a is an integer of 4 to 6, and is linked to the benzene ring in a similar manner, whereas the indication of hydrogen bound to the carbon forming the benzene ring is omitted.

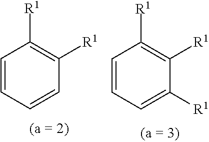

Hereinafter, a compound according to an aspect of the present invention and an organic electric element comprising the same will be described.

The present invention provides a compound represented Formula (1) below.

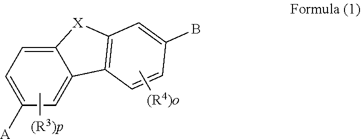

{In Formula (1),
1) A and B are each selected from the group of Formula(1-1), (1-2) and are different,

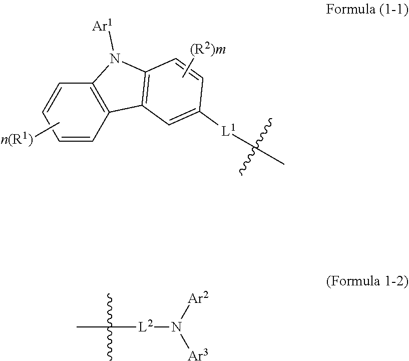

2) Ar¹, Ar² and Ar³ are each independently selected from the group consisting of a C₆-C₆₀ aryl group; a fluorenyl group; a C₂-C₆₀ heterocyclic group including at least one hetero atom of O, N, S, Si or P; a fused ring group of a C₃-C₆₀ aliphatic ring and a C₆-C₆₀ aromatic ring;
3) X is O or S,
4) L¹ and L² are independently selected from the group consisting of a single bond; a C₆-C₆₀ arylene group; and a fluorenylene group; a fused ring group of a C₃-C₆₀ aliphatic ring and a C₆-C₆₀ aromatic ring; and a C₂-C₆₀ heterocyclic group;

5) n is an integer of 0 to 4, and m, p and o are an integer of 0 to 3, when m, n, o or p are 1 or more, R¹, R², R³, and R⁴ are each independently selected from a hydrogen; a deuterium; a halogen; the group consisting of a C₆-C₆₀ aryl group; a fluorenyl group; a C₂-C₆₀ heterocyclic group including at least one hetero atom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; and -L′-N(Rₐ)(R_{b});(wherein, L′ is selected from the group consisting of a single bond; a C₆-C₆₀ arylene group; a fluorenylene group; a fused ring group of a C₃-C₆₀ aliphatic ring and a C₆-C₆₀ aromatic ring; and a C₂-C₆₀ heterocyclic, and Rₐ and R_{b} are be independently selected from the group consisting of a C₆-C₆₀ aryl group; a fluorenyl group; a fused ring group of a C₃-C₆₀ aliphatic ring and a C₆-C₆₀ aromatic ring; and a C₂-C₆₀ heterocyclic group containing at least one hetero atom of O, N, S, Si, or P), or an adjacent plurality of R¹ or a plurality of R² or a plurality of R³ or a plurality of R⁴ may be bonded to each other to form an aromatic or a heteroaromatic ring.

wherein, the aryl group, fluorenyl group, arylene group, heterocyclic group, fused ring group, alkyl group, alkenyl group, alkoxy group and aryloxy group may be substituted with one or more substituents selected from the group consisting of deuterium; halogen; a silane group substituted or unsubstituted with C₁-C₂₀ alkyl group or C₆-C₂₀ aryl group; siloxane group; boron group; germanium group; cyano group; nitro group; -L′-N(Rₐ)(R_{b}); a C₁-C₂₀ alkylthio group; C₁-C₂₀ alkoxyl group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂-C₂₀ heterocyclic group; C₃-C₂₀ cycloalkyl group; C₇-C₂₀ arylalkyl group and C₈-C₂₀ arylalkenyl group, and also, these substituents may combine each other and form a ring, wherein the term ‘ring’ means C₃-C₆₀ aliphatic ring or C₆-C₆₀ aromatic ring or a C₂-C₆₀ heterocyclic ring or a fused ring formed by the combination of thereof and comprises a saturated or unsaturated ring.}

Also, in one embodiment of the present invention, the compound represented by Formula (1) includes a compound represented by the following Formula (2) or (3).

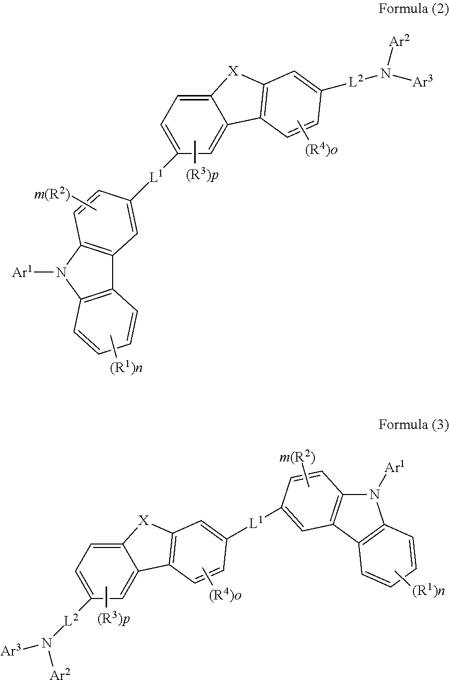

(In Formula (2) or Formula (3),
R¹, R², R³, R⁴, m, n, o, p, L¹, L², Ar¹, Ar², and Ar³ are the same as defined in Formula (1).)

As specific examples of the compound of the present invention, the compound represented by Formula (1) includes compounds represented by the following formulas.

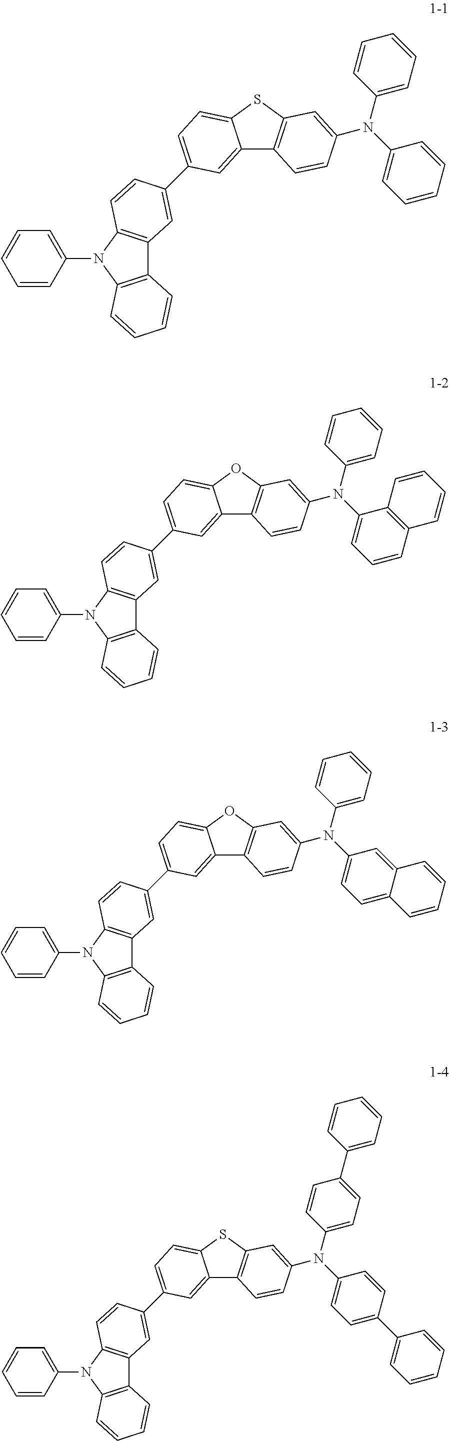 [SMILES: c1ccc(N(c2ccccc2)c2ccc3c(c2)sc2ccc(-c4ccc5c(c4)c4ccccc4n5-c4ccccc4)cc23)cc1 ; c1ccc(N(c2ccc3c(c2)oc2ccc(-c4ccc5c(c4)c4ccccc4n5-c4ccccc4)cc23)c2cccc3ccccc23)cc1 ; c1ccc(N(c2ccc3ccccc3c2)c2ccc3c(c2)oc2ccc(-c4ccc5c(c4)c4ccccc4n5-c4ccccc4)cc23)cc1 ; c1ccc(-c2ccc(N(c3ccc(-c4ccccc4)cc3)c3ccc4c(c3)sc3ccc(-c5ccc6c(c5)c5ccccc5n6-c5ccccc5)cc34)cc2)cc1]

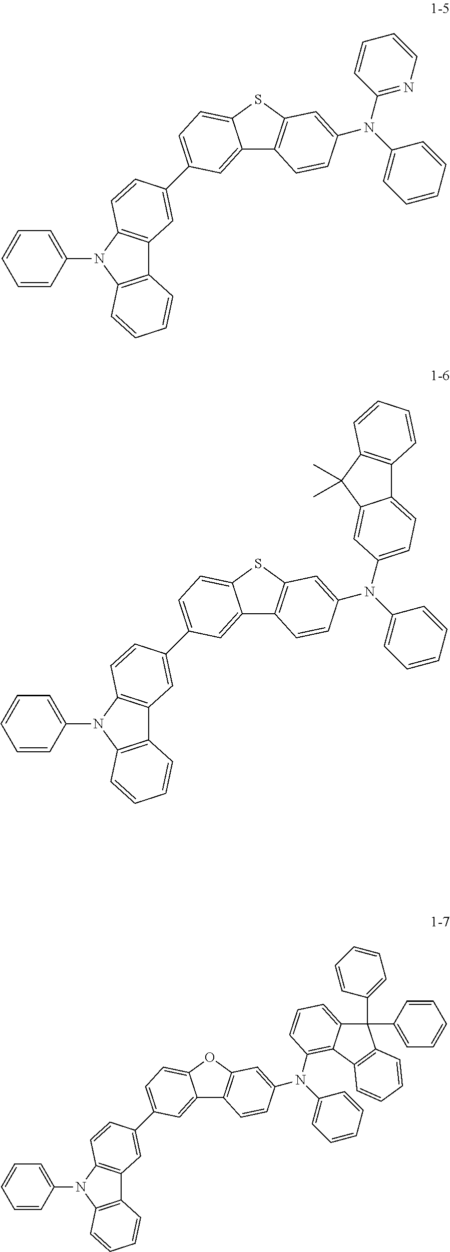 [SMILES: c1ccc(N(c2ccc3c(c2)sc2ccc(-c4ccc5c(c4)c4ccccc4n5-c4ccccc4)cc23)c2ccccn2)cc1 ; CC1(C)c2ccccc2-c2ccc(N(c3ccccc3)c3ccc4c(c3)sc3ccc(-c5ccc6c(c5)c5ccccc5n6-c5ccccc5)cc34)cc21 ; c1ccc(N(c2ccc3c(c2)oc2ccc(-c4ccc5c(c4)c4ccccc4n5-c4ccccc4)cc23)c2cccc3c2-c2ccccc2C3(c2ccccc2)c2ccccc2)cc1]

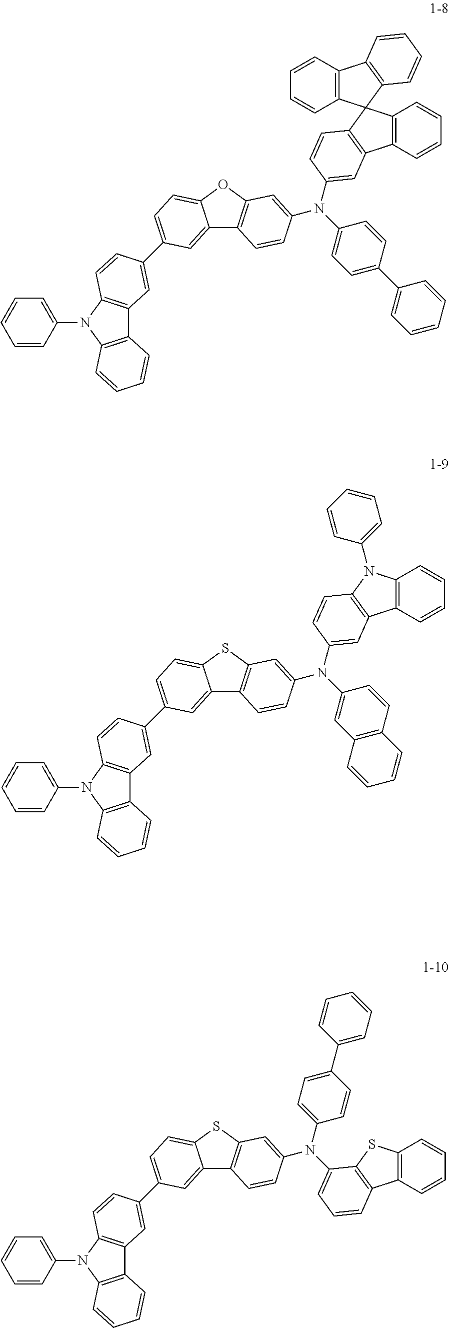 [SMILES: c1ccc(-c2ccc(N(c3ccc4c(c3)-c3ccccc3C43c4ccccc4-c4ccccc43)c3ccc4c(c3)oc3ccc(-c5ccc6c(c5)c5ccccc5n6-c5ccccc5)cc34)cc2)cc1 ; c1ccc(-n2c3ccccc3c3cc(-c4ccc5sc6cc(N(c7ccc8ccccc8c7)c7ccc8c(c7)c7ccccc7n8-c7ccccc7)ccc6c5c4)ccc32)cc1 ; c1ccc(-c2ccc(N(c3ccc4c(c3)sc3ccc(-c5ccc6c(c5)c5ccccc5n6-c5ccccc5)cc34)c3cccc4c3sc3ccccc34)cc2)cc1]

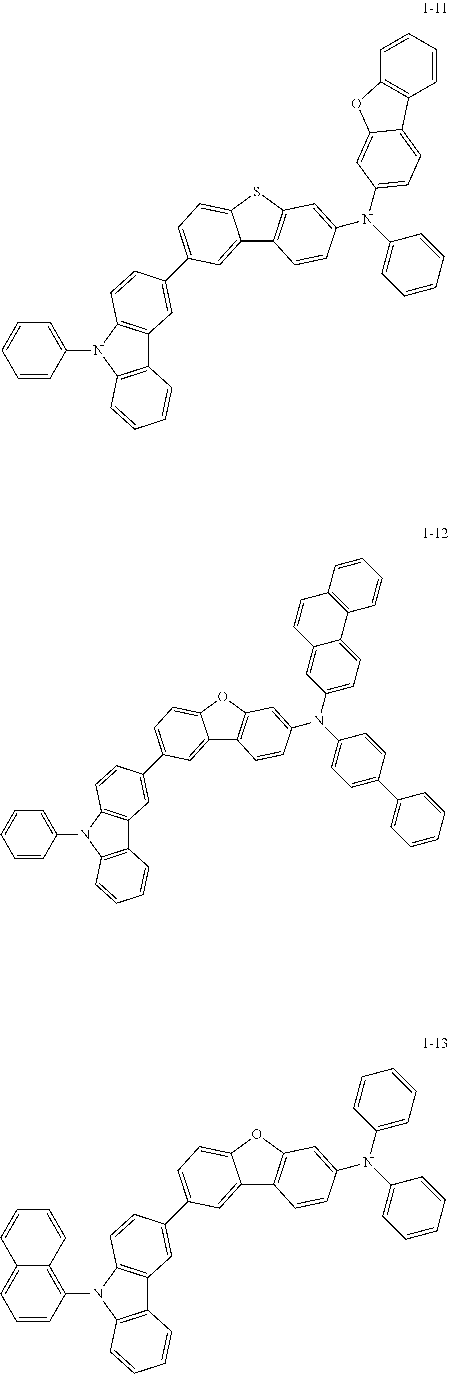 [SMILES: c1ccc(N(c2ccc3c(c2)oc2ccccc23)c2ccc3c(c2)sc2ccc(-c4ccc5c(c4)c4ccccc4n5-c4ccccc4)cc23)cc1 ; c1ccc(-c2ccc(N(c3ccc4c(ccc5ccccc54)c3)c3ccc4c(c3)oc3ccc(-c5ccc6c(c5)c5ccccc5n6-c5ccccc5)cc34)cc2)cc1 ; c1ccc(N(c2ccccc2)c2ccc3c(c2)oc2ccc(-c4ccc5c(c4)c4ccccc4n5-c4cccc5ccccc45)cc23)cc1]

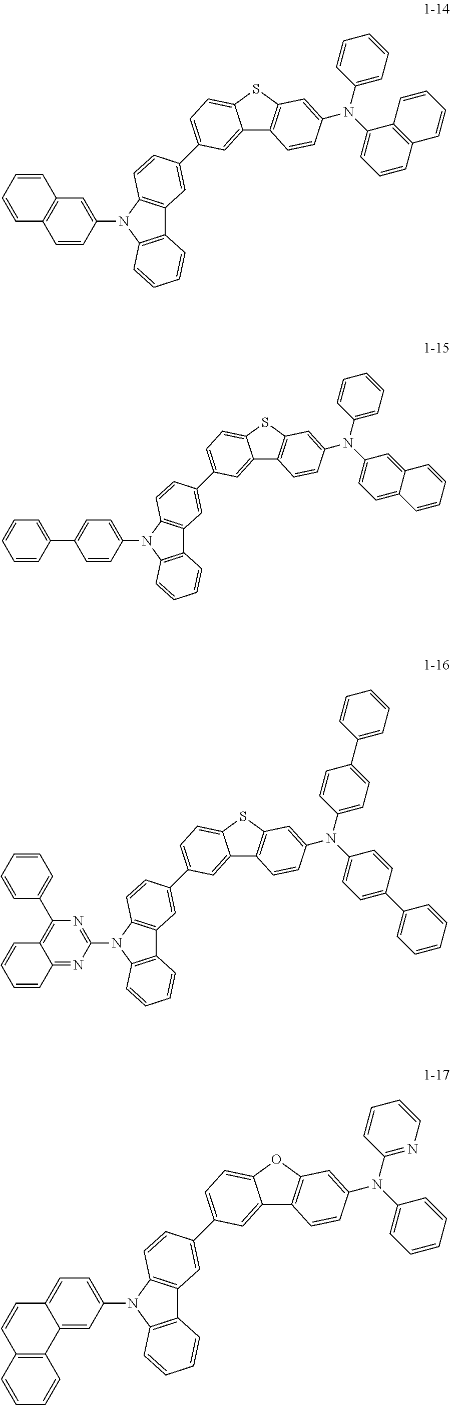 [SMILES: c1ccc(N(c2ccc3c(c2)sc2ccc(-c4ccc5c(c4)c4ccccc4n5-c4ccc5ccccc5c4)cc23)c2cccc3ccccc23)cc1 ; c1ccc(-c2ccc(-n3c4ccccc4c4cc(-c5ccc6sc7cc(N(c8ccccc8)c8ccc9ccccc9c8)ccc7c6c5)ccc43)cc2)cc1 ; c1ccc(-c2ccc(N(c3ccc(-c4ccccc4)cc3)c3ccc4c(c3)sc3ccc(-c5ccc6c(c5)c5ccccc5n6-c5nc(-c6ccccc6)c6ccccc6n5)cc34)cc2)cc1 ; c1ccc(N(c2ccc3c(c2)oc2ccc(-c4ccc5c(c4)c4ccccc4n5-c4ccc5ccc6ccccc6c5c4)cc23)c2ccccn2)cc1]

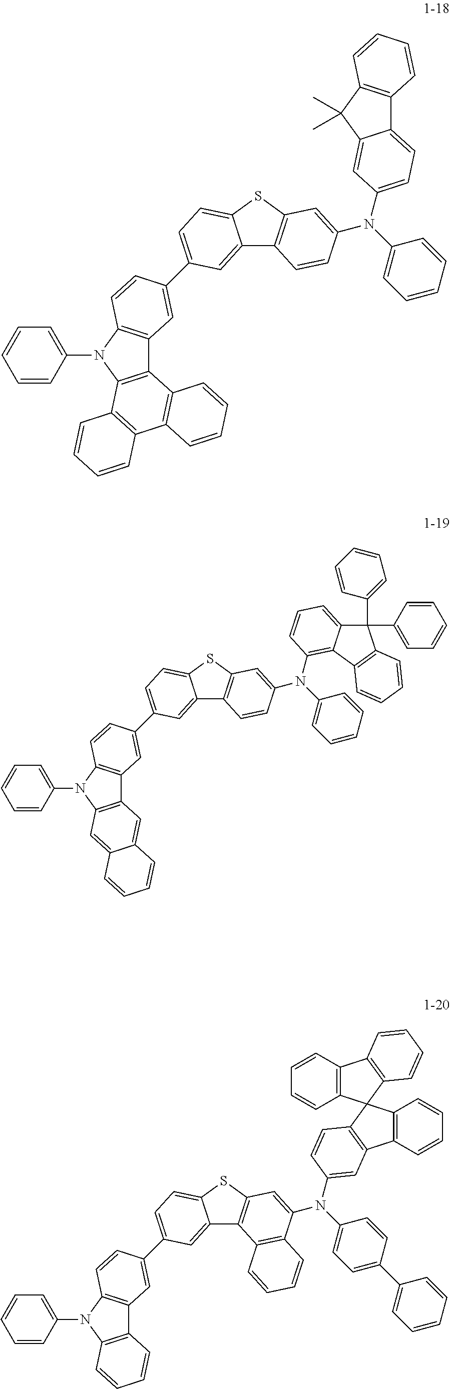 [SMILES: CC1(C)c2ccccc2-c2ccc(N(c3ccccc3)c3ccc4c(c3)sc3ccc(-c5ccc6c(c5)c5c7ccccc7c7ccccc7c5n6-c5ccccc5)cc34)cc21 ; c1ccc(N(c2ccc3c(c2)sc2ccc(-c4ccc5c(c4)c4cc6ccccc6cc4n5-c4ccccc4)cc23)c2cccc3c2-c2ccccc2C3(c2ccccc2)c2ccccc2)cc1 ; c1ccc(-c2ccc(N(c3ccc4c(c3)-c3ccccc3C43c4ccccc4-c4ccccc43)c3cc4sc5ccc(-c6ccc7c(c6)c6ccccc6n7-c6ccccc6)cc5c4c4ccccc34)cc2)cc1]

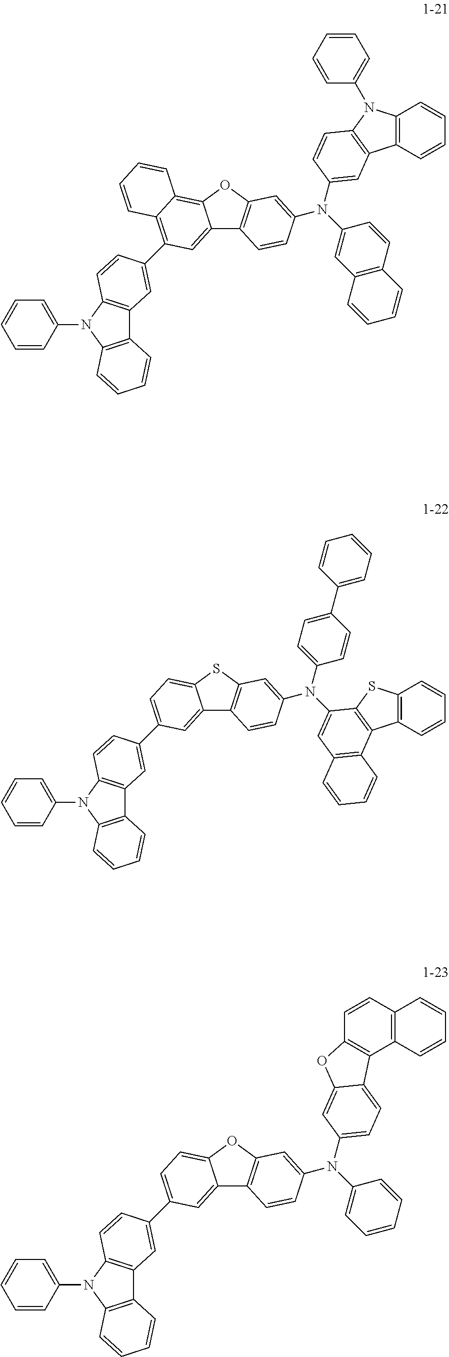 [SMILES: c1ccc(-n2c3ccccc3c3cc(-c4cc5c6ccc(N(c7ccc8ccccc8c7)c7ccc8c(c7)c7ccccc7n8-c7ccccc7)cc6oc5c5ccccc45)ccc32)cc1 ; c1ccc(-c2ccc(N(c3ccc4c(c3)sc3ccc(-c5ccc6c(c5)c5ccccc5n6-c5ccccc5)cc34)c3cc4ccccc4c4c3sc3ccccc34)cc2)cc1 ; c1ccc(N(c2ccc3c(c2)oc2ccc(-c4ccc5c(c4)c4ccccc4n5-c4ccccc4)cc23)c2ccc3c(c2)oc2ccc4ccccc4c23)cc1]

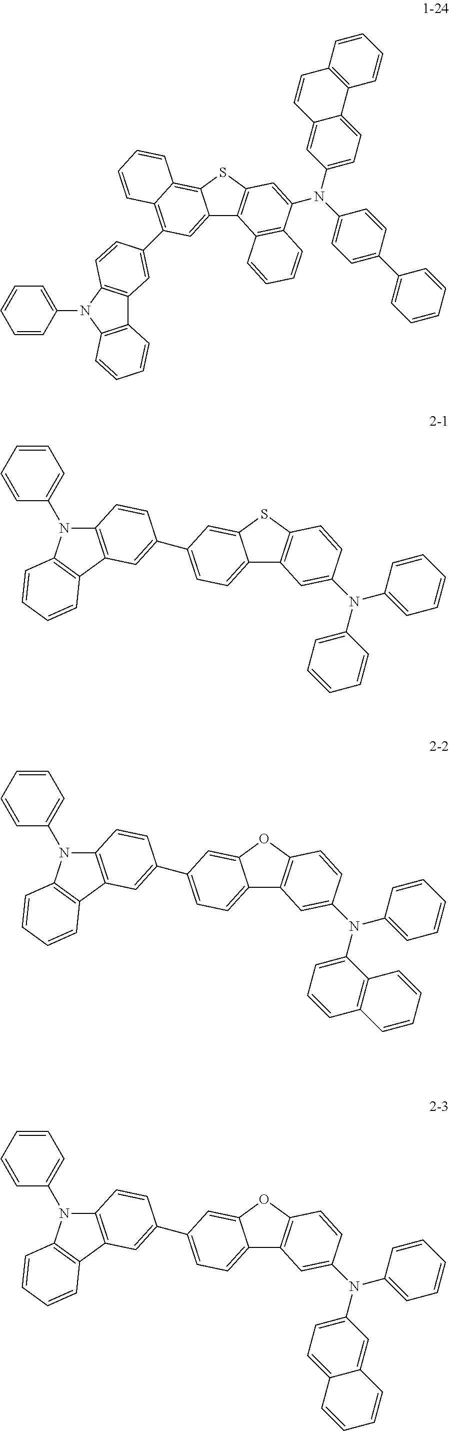 [SMILES: c1ccc(-c2ccc(N(c3ccc4c(ccc5ccccc54)c3)c3cc4sc5c6ccccc6c(-c6ccc7c(c6)c6ccccc6n7-c6ccccc6)cc5c4c4ccccc34)cc2)cc1 ; c1ccc(N(c2ccccc2)c2ccc3sc4cc(-c5ccc6c(c5)c5ccccc5n6-c5ccccc5)ccc4c3c2)cc1 ; c1ccc(N(c2ccc3oc4cc(-c5ccc6c(c5)c5ccccc5n6-c5ccccc5)ccc4c3c2)c2cccc3ccccc23)cc1 ; c1ccc(N(c2ccc3ccccc3c2)c2ccc3oc4cc(-c5ccc6c(c5)c5ccccc5n6-c5ccccc5)ccc4c3c2)cc1]

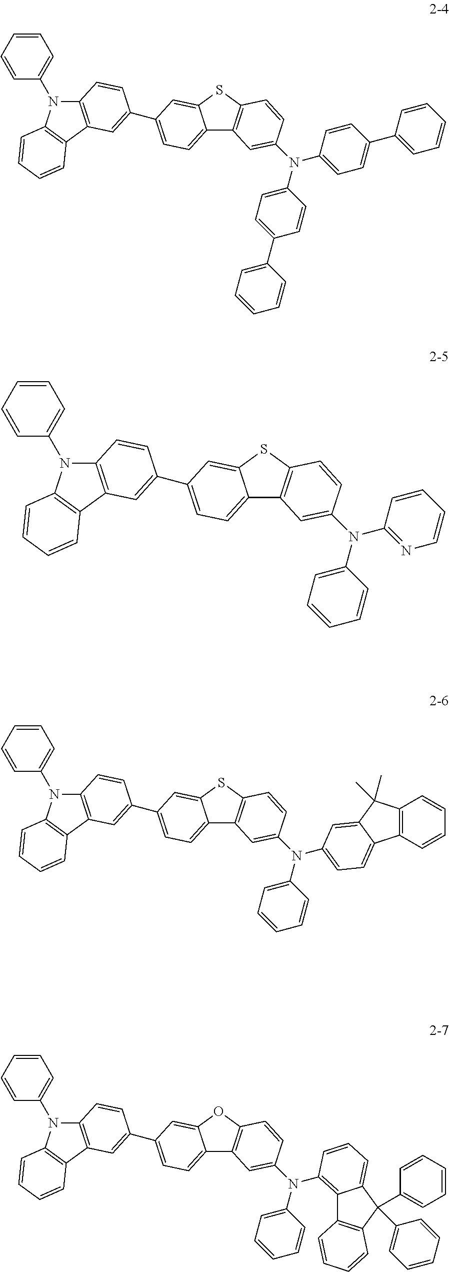 [SMILES: c1ccc(-c2ccc(N(c3ccc(-c4ccccc4)cc3)c3ccc4sc5cc(-c6ccc7c(c6)c6ccccc6n7-c6ccccc6)ccc5c4c3)cc2)cc1 ; c1ccc(N(c2ccc3sc4cc(-c5ccc6c(c5)c5ccccc5n6-c5ccccc5)ccc4c3c2)c2ccccn2)cc1 ; CC1(C)c2ccccc2-c2ccc(N(c3ccccc3)c3ccc4sc5cc(-c6ccc7c(c6)c6ccccc6n7-c6ccccc6)ccc5c4c3)cc21 ; c1ccc(N(c2ccc3oc4cc(-c5ccc6c(c5)c5ccccc5n6-c5ccccc5)ccc4c3c2)c2cccc3c2-c2ccccc2C3(c2ccccc2)c2ccccc2)cc1]

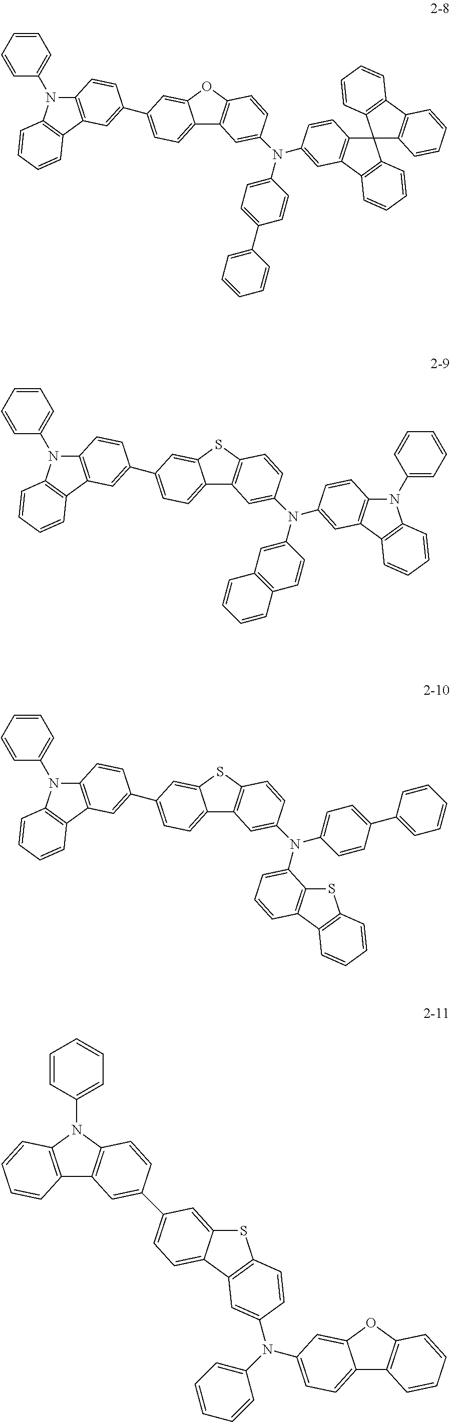 [SMILES: c1ccc(-c2ccc(N(c3ccc4c(c3)-c3ccccc3C43c4ccccc4-c4ccccc43)c3ccc4oc5cc(-c6ccc7c(c6)c6ccccc6n7-c6ccccc6)ccc5c4c3)cc2)cc1 ; c1ccc(-n2c3ccccc3c3cc(-c4ccc5c(c4)sc4ccc(N(c6ccc7ccccc7c6)c6ccc7c(c6)c6ccccc6n7-c6ccccc6)cc45)ccc32)cc1 ; c1ccc(-c2ccc(N(c3ccc4sc5cc(-c6ccc7c(c6)c6ccccc6n7-c6ccccc6)ccc5c4c3)c3cccc4c3sc3ccccc34)cc2)cc1 ; c1ccc(N(c2ccc3c(c2)oc2ccccc23)c2ccc3sc4cc(-c5ccc6c(c5)c5ccccc5n6-c5ccccc5)ccc4c3c2)cc1]

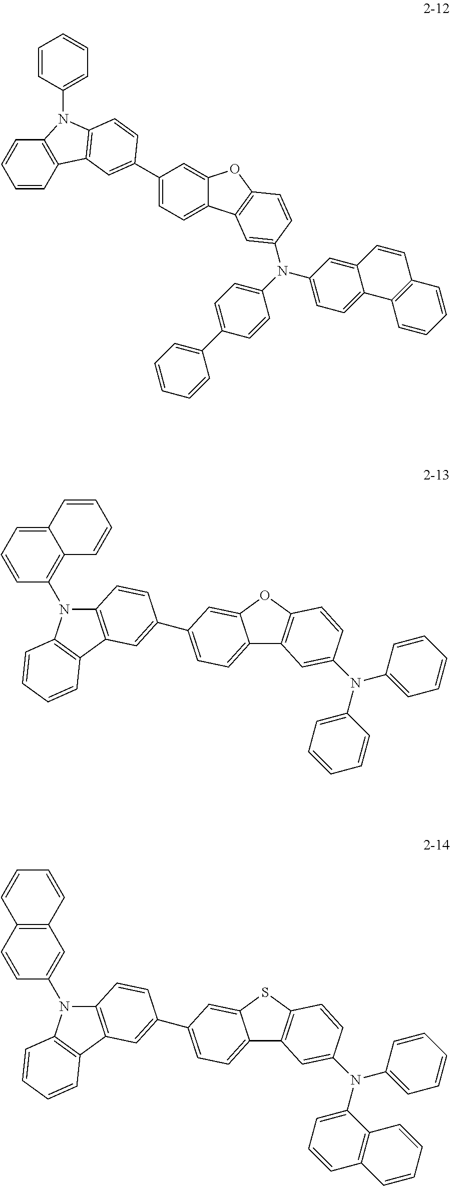 [SMILES: c1ccc(-c2ccc(N(c3ccc4c(ccc5ccccc54)c3)c3ccc4oc5cc(-c6ccc7c(c6)c6ccccc6n7-c6ccccc6)ccc5c4c3)cc2)cc1 ; c1ccc(N(c2ccccc2)c2ccc3oc4cc(-c5ccc6c(c5)c5ccccc5n6-c5cccc6ccccc56)ccc4c3c2)cc1 ; c1ccc(N(c2ccc3sc4cc(-c5ccc6c(c5)c5ccccc5n6-c5ccc6ccccc6c5)ccc4c3c2)c2cccc3ccccc23)cc1]

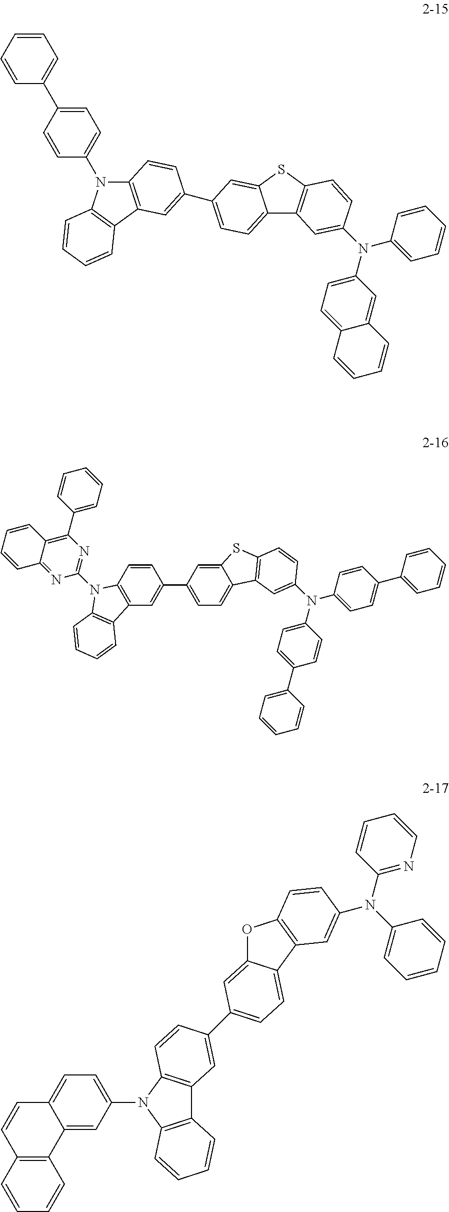 [SMILES: c1ccc(-c2ccc(-n3c4ccccc4c4cc(-c5ccc6c(c5)sc5ccc(N(c7ccccc7)c7ccc8ccccc8c7)cc56)ccc43)cc2)cc1 ; c1ccc(-c2ccc(N(c3ccc(-c4ccccc4)cc3)c3ccc4sc5cc(-c6ccc7c(c6)c6ccccc6n7-c6nc(-c7ccccc7)c7ccccc7n6)ccc5c4c3)cc2)cc1 ; c1ccc(N(c2ccc3oc4cc(-c5ccc6c(c5)c5ccccc5n6-c5ccc6ccc7ccccc7c6c5)ccc4c3c2)c2ccccn2)cc1]

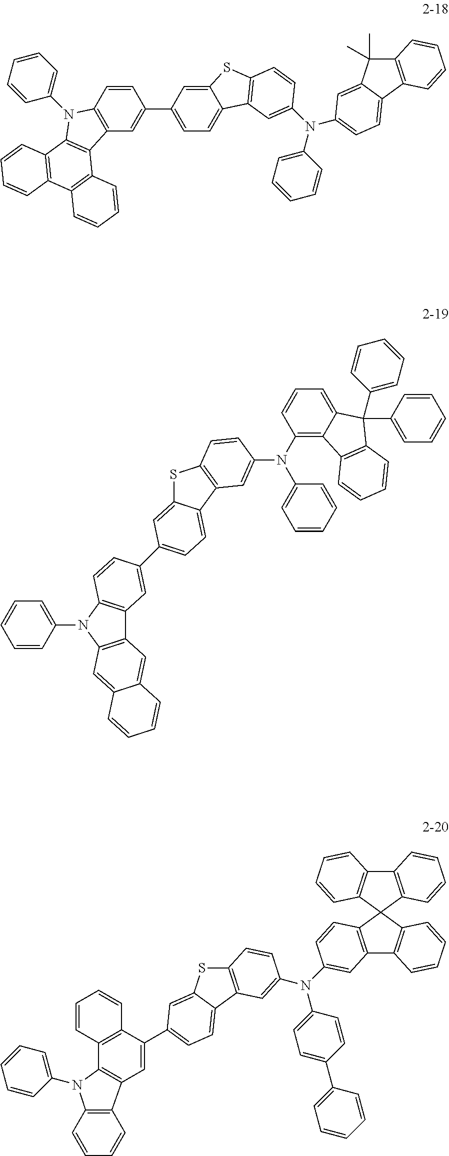 [SMILES: CC1(C)c2ccccc2-c2ccc(N(c3ccccc3)c3ccc4sc5cc(-c6ccc7c(c6)c6c8ccccc8c8ccccc8c6n7-c6ccccc6)ccc5c4c3)cc21 ; c1ccc(N(c2ccc3sc4cc(-c5ccc6c(c5)c5cc7ccccc7cc5n6-c5ccccc5)ccc4c3c2)c2cccc3c2-c2ccccc2C3(c2ccccc2)c2ccccc2)cc1 ; c1ccc(-c2ccc(N(c3ccc4c(c3)-c3ccccc3C43c4ccccc4-c4ccccc43)c3ccc4sc5cc(-c6cc7c8ccccc8n(-c8ccccc8)c7c7ccccc67)ccc5c4c3)cc2)cc1]

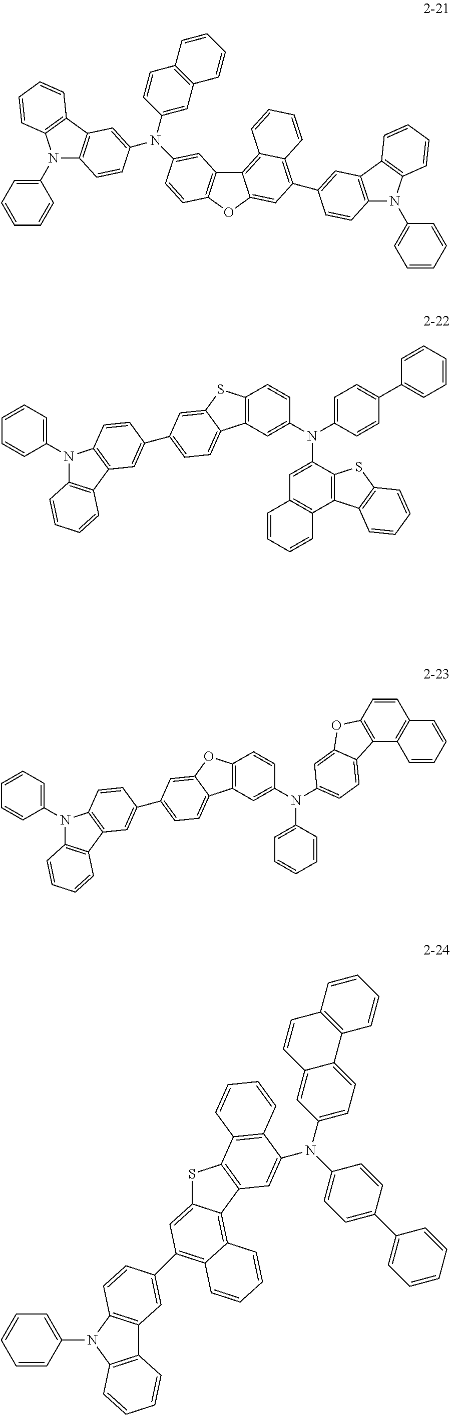 [SMILES: c1ccc(-n2c3ccccc3c3cc(-c4cc5oc6ccc(N(c7ccc8ccccc8c7)c7ccc8c(c7)c7ccccc7n8-c7ccccc7)cc6c5c5ccccc45)ccc32)cc1 ; c1ccc(-c2ccc(N(c3ccc4sc5cc(-c6ccc7c(c6)c6ccccc6n7-c6ccccc6)ccc5c4c3)c3cc4ccccc4c4c3sc3ccccc34)cc2)cc1 ; c1ccc(N(c2ccc3c(c2)oc2ccc4ccccc4c23)c2ccc3oc4cc(-c5ccc6c(c5)c5ccccc5n6-c5ccccc5)ccc4c3c2)cc1 ; c1ccc(-c2ccc(N(c3ccc4c(ccc5ccccc54)c3)c3cc4c(sc5cc(-c6ccc7c(c6)c6ccccc6n7-c6ccccc6)c6ccccc6c54)c4ccccc34)cc2)cc1]

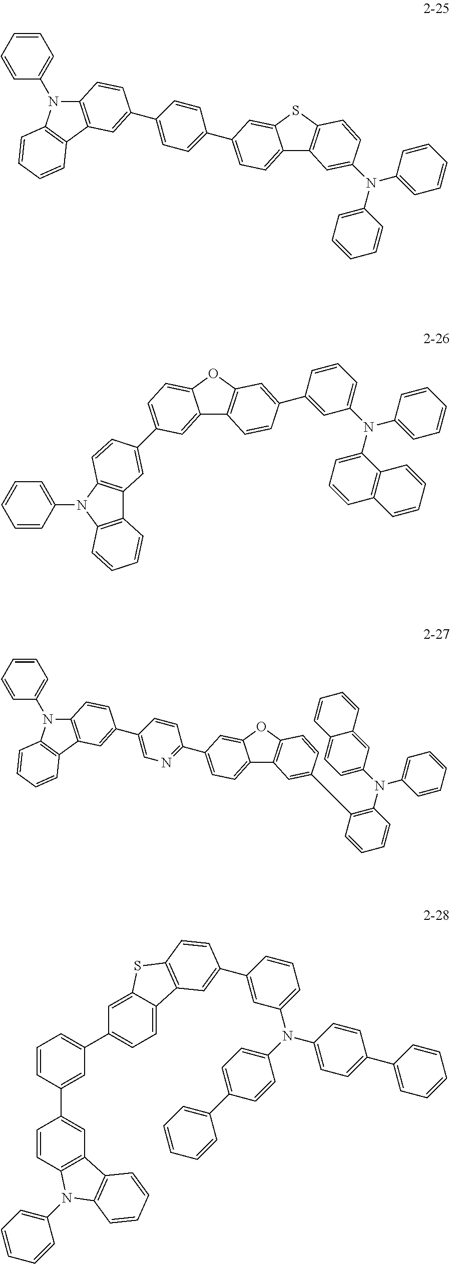 [SMILES: c1ccc(N(c2ccccc2)c2ccc3sc4cc(-c5ccc(-c6ccc7c(c6)c6ccccc6n7-c6ccccc6)cc5)ccc4c3c2)cc1 ; c1ccc(N(c2cccc(-c3ccc4c(c3)oc3ccc(-c5ccc6c(c5)c5ccccc5n6-c5ccccc5)cc34)c2)c2cccc3ccccc23)cc1 ; c1ccc(N(c2ccc3ccccc3c2)c2ccccc2-c2ccc3oc4cc(-c5ccc(-c6ccc7c(c6)c6ccccc6n7-c6ccccc6)cn5)ccc4c3c2)cc1 ; c1ccc(-c2ccc(N(c3ccc(-c4ccccc4)cc3)c3cccc(-c4ccc5sc6cc(-c7cccc(-c8ccc9c(c8)c8ccccc8n9-c8ccccc8)c7)ccc6c5c4)c3)cc2)cc1]

The present invention provides an organic electric element comprising a first electrode, a second electrode and an organic material layer formed between the first electrode and the second electrode, wherein the organic material layer comprises an hole injection layer, an hole transport layer, an emitting auxiliary layer and an emitting layer, wherein the organic material layer provides a compound represented by Formula (1).

Also, the present invention provides an organic electric element comprising a first electrode, a second electrode and an organic material layer formed between the first electrode and the second electrode, wherein the organic electric element comprises an hole transport layer formed between the first electrode and the emitting layer, and an emitting auxiliary layer formed between the hole transport layer and the emitting layer, wherein the emitting auxiliary layer includes a compound represented by Formula (1), and wherein the hole transport layer includes a compound represented by Formula (5).

Also, the present invention provides an organic electric element comprising the compound represented by Formula (1) alone or a mixture of two or more compounds having different structures in at least one of the hole injection layer, the hole transport layer, the emitting auxiliary layer and the emitting layer. Specifically, the hole transport layer or the emitting auxiliary layer comprises the compound alone or a mixture of two or more compounds having different structures.

Also, the emitting auxiliary layer comprises a compound represented by Formula (1), and the hole transport layer comprises a compound represented by Formula (5)

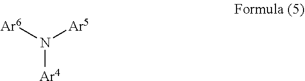

{In Formula (5),
1) Ar⁴ and Ar⁵ are each independently selected from the group consisting of a C₆-C₆₀ aryl group; a fluorenyl group; a C₂-C₆₀ heterocyclic group including at least one hetero atom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; and -L′-N(Rₐ)(R_{b})(where, L′ may be selected from the group consisting of a single bond; a C₆-C₆₀ arylene group; a fluorenylene group; a fused ring group of a C₃-C₆₀ aliphatic ring and a C₆-C₆₀ aromatic ring; and a C₂-C₆₀ heterocyclic, and the Rₐ and R_{b} may be independently selected from the group consisting of a C₆-C₆₀ aryl group; a fluorenyl group; a fused ring group of a C₃-C₆₀ aliphatic ring and a C₆-C₆₀ aromatic ring; and a C₂-C₆₀ heterocyclic group containing at least one hetero atom of O, N, S, Si, or P), or Ar⁴ and Ar⁵ may be bonded to each other to form a ring,

2) Ar⁶ is selected from any one of the following Formulas (5-a), (5-b) and (5-c)

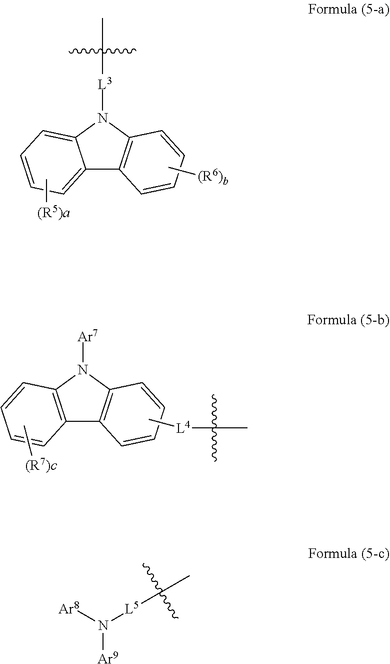

3) a, b, and c are integers of 0 to 4, and R⁵, R⁶ and R⁷ are the same or different and are each independently selected from a deuterium; a halogen; the group consisting of a C₆-C₆₀ aryl group; a fluorenyl group; a C₂-C₆₀ heterocyclic group including at least one hetero atom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; and -L′-N(Rₐ)(R_{b}); or in case a, b and c are 2 or more, and R⁵, R⁶ and R⁷ are each in plural and are the same or different, or a plurality of R⁵ or a plurality of R⁶ or a plurality of R⁷ may be bonded to each other to form a ring,

4) L³ and L⁵ are each independently selected from the group consisting of a C₆-C₆₀ arylene group; a fluorenylene group; a fused ring group of a C₃-C₆₀ aliphatic ring and a C₆-C₆₀ aromatic ring; and a C₂-C₆₀ heterocyclic group including at least one hetero atom of O, N, S, Si or P;

5) L⁴ is selected from a single bond of C₆-C₆₀; an arylene group; a fluorenylene group; a fused ring group of a C₃-C₆₀ aliphatic ring and a C₆-C₆₀ aromatic ring; and a C₂-C₆₀ heterocyclic group including at least one hetero atom of O, N, S, Si or P;

6) Ar⁷, Ar⁸ and Ar⁹ are each independently selected from the group consisting of a C₆-C₆₀ aryl group; a fluorenyl group; a C₂-C₆₀ heterocyclic group including at least one hetero atom of O, N, S, Si or P; a fused ring group of a C₃-C₆₀ aliphatic ring and a C₆-C₆₀ aromatic ring;

Here, the aryl group, the fluorenyl group, the arylene group, the heterocyclic group, the fused ring group, the alkyl group, the alkenyl group, the alkoxy group and the aryloxy group may be each substituted with one or more substituents selected from a group consisting of a deuterium; halogen; a silane group substituted or unsubstituted with a C₁-C₂₀ alkyl group or a C₆-C₆₀ aryl group; a siloxane group; a boron group; a germanium group; a cyano; a nitro group; -L′-N(Rₐ)(R_{b}); a C₁-C₂₀ alkylthio group; C₁-C₂₀ alkoxyl group; C₁-C₂₀ alkyl group; C₂-C₂₀ group; C₂-C₂₀ alkynyl group; C₆-C₂₀ aryl group; C₆-C₂₀ aryl group substituted with deuterium; a fluorenyl group; C₂-C₂₀ heterocyclic group; C₃-C₂₀ cycloalkyl group; C₇-C₂₀ arylalkyl group and C₈-C₂₀ arylalkenyl group, and also, these substituents may be bonded to each other to form a ring, wherein the term ‘ring’ means C₃-C₆₀ aliphatic ring or C₆-C₆₀ aromatic ring or a C₂-C₆₀ heterocyclic ring or a fused ring formed by the combination of them, and includes a saturated or unsaturated ring.}

In the present invention, the compound represented by Formula (5) includes the compounds represented by the following Formulas (5-1) to (5-71).

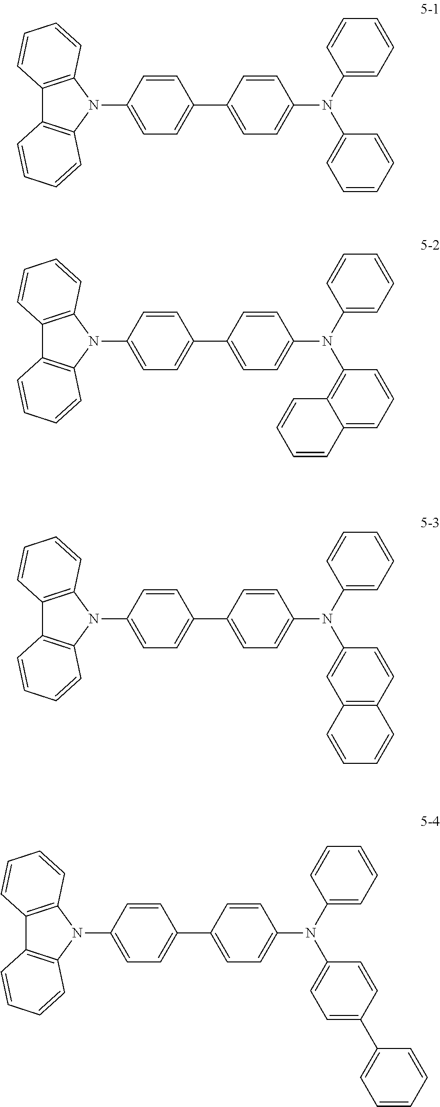 [SMILES: c1ccc(N(c2ccccc2)c2ccc(-c3ccc(-n4c5ccccc5c5ccccc54)cc3)cc2)cc1 ; c1ccc(N(c2ccc(-c3ccc(-n4c5ccccc5c5ccccc54)cc3)cc2)c2cccc3ccccc23)cc1 ; c1ccc(N(c2ccc(-c3ccc(-n4c5ccccc5c5ccccc54)cc3)cc2)c2ccc3ccccc3c2)cc1 ; c1ccc(-c2ccc(N(c3ccccc3)c3ccc(-c4ccc(-n5c6ccccc6c6ccccc65)cc4)cc3)cc2)cc1]

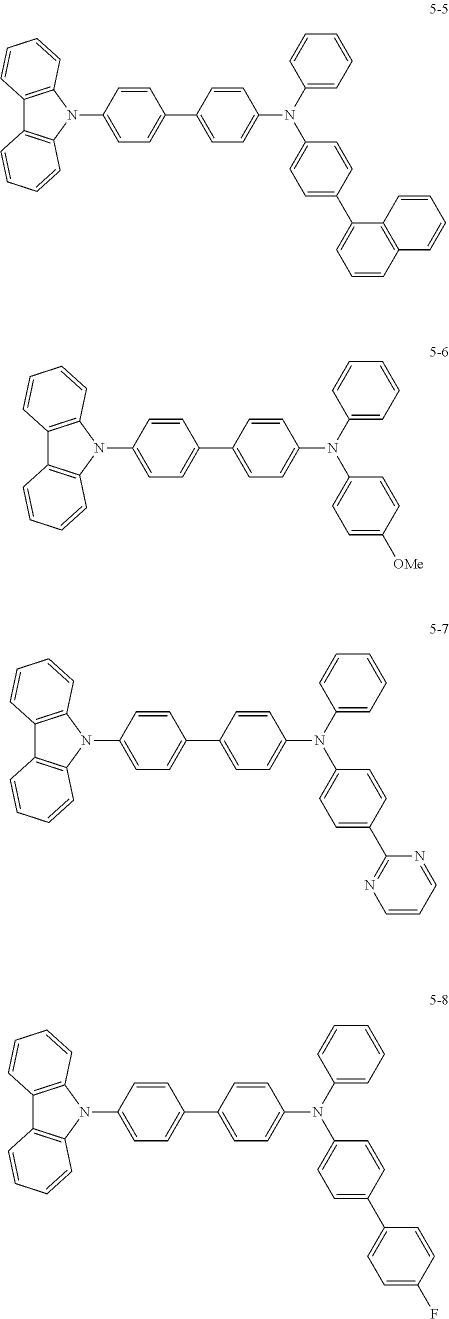 [SMILES: c1ccc(N(c2ccc(-c3ccc(-n4c5ccccc5c5ccccc54)cc3)cc2)c2ccc(-c3cccc4ccccc34)cc2)cc1 ; COc1ccc(N(c2ccccc2)c2ccc(-c3ccc(-n4c5ccccc5c5ccccc54)cc3)cc2)cc1 ; c1ccc(N(c2ccc(-c3ccc(-n4c5ccccc5c5ccccc54)cc3)cc2)c2ccc(-c3ncccn3)cc2)cc1 ; Fc1ccc(-c2ccc(N(c3ccccc3)c3ccc(-c4ccc(-n5c6ccccc6c6ccccc65)cc4)cc3)cc2)cc1]

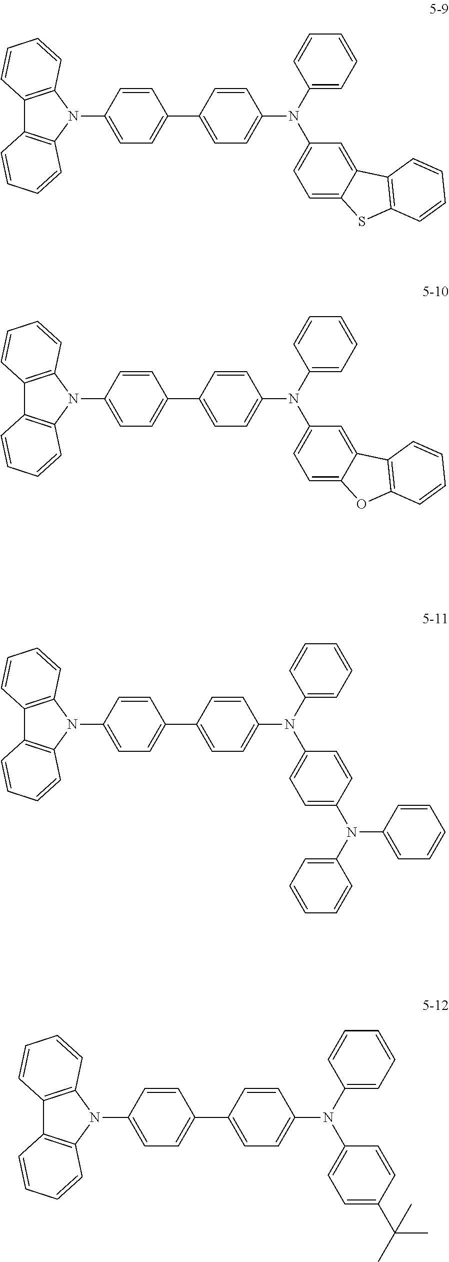 [SMILES: c1ccc(N(c2ccc(-c3ccc(-n4c5ccccc5c5ccccc54)cc3)cc2)c2ccc3sc4ccccc4c3c2)cc1 ; c1ccc(N(c2ccc(-c3ccc(-n4c5ccccc5c5ccccc54)cc3)cc2)c2ccc3oc4ccccc4c3c2)cc1 ; c1ccc(N(c2ccccc2)c2ccc(N(c3ccccc3)c3ccc(-c4ccc(-n5c6ccccc6c6ccccc65)cc4)cc3)cc2)cc1 ; CC(C)(C)c1ccc(N(c2ccccc2)c2ccc(-c3ccc(-n4c5ccccc5c5ccccc54)cc3)cc2)cc1]

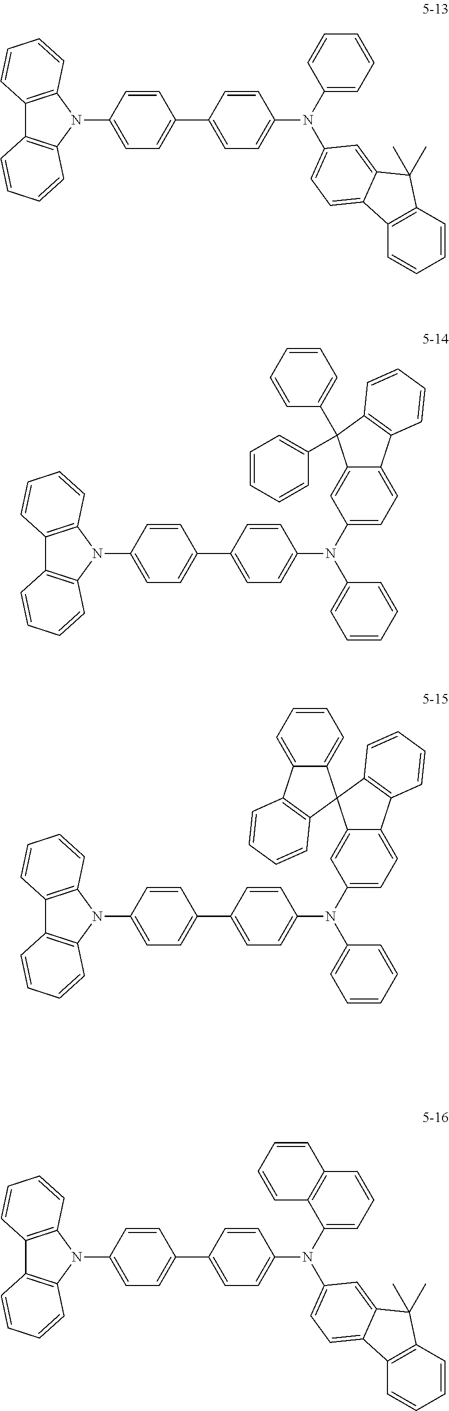 [SMILES: CC1(C)c2ccccc2-c2ccc(N(c3ccccc3)c3ccc(-c4ccc(-n5c6ccccc6c6ccccc65)cc4)cc3)cc21 ; c1ccc(N(c2ccc(-c3ccc(-n4c5ccccc5c5ccccc54)cc3)cc2)c2ccc3c(c2)C(c2ccccc2)(c2ccccc2)c2ccccc2-3)cc1 ; c1ccc(N(c2ccc(-c3ccc(-n4c5ccccc5c5ccccc54)cc3)cc2)c2ccc3c(c2)C2(c4ccccc4-c4ccccc42)c2ccccc2-3)cc1 ; CC1(C)c2ccccc2-c2ccc(N(c3ccc(-c4ccc(-n5c6ccccc6c6ccccc65)cc4)cc3)c3cccc4ccccc34)cc21]

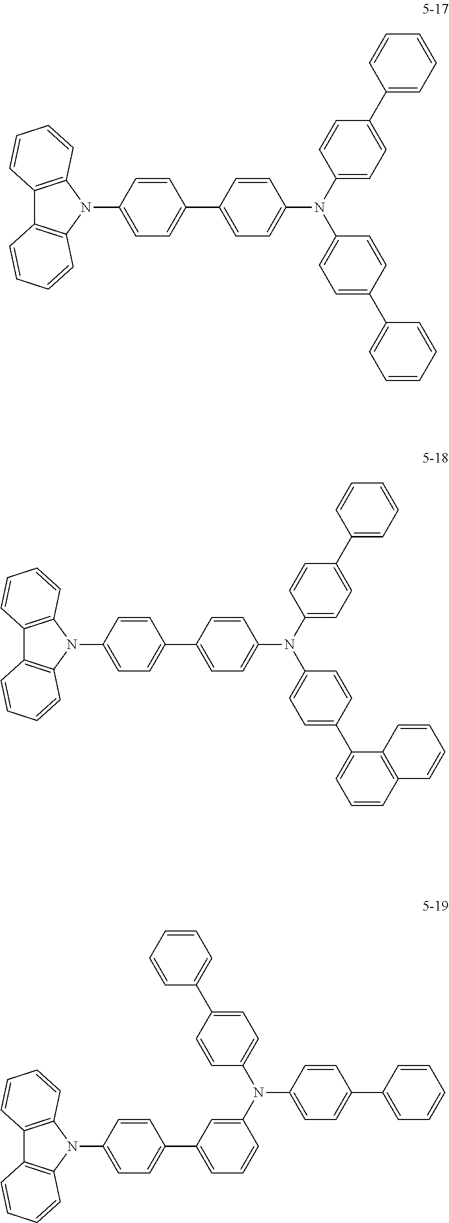 [SMILES: c1ccc(-c2ccc(N(c3ccc(-c4ccccc4)cc3)c3ccc(-c4ccc(-n5c6ccccc6c6ccccc65)cc4)cc3)cc2)cc1 ; c1ccc(-c2ccc(N(c3ccc(-c4ccc(-n5c6ccccc6c6ccccc65)cc4)cc3)c3ccc(-c4cccc5ccccc45)cc3)cc2)cc1 ; c1ccc(-c2ccc(N(c3ccc(-c4ccccc4)cc3)c3cccc(-c4ccc(-n5c6ccccc6c6ccccc65)cc4)c3)cc2)cc1]

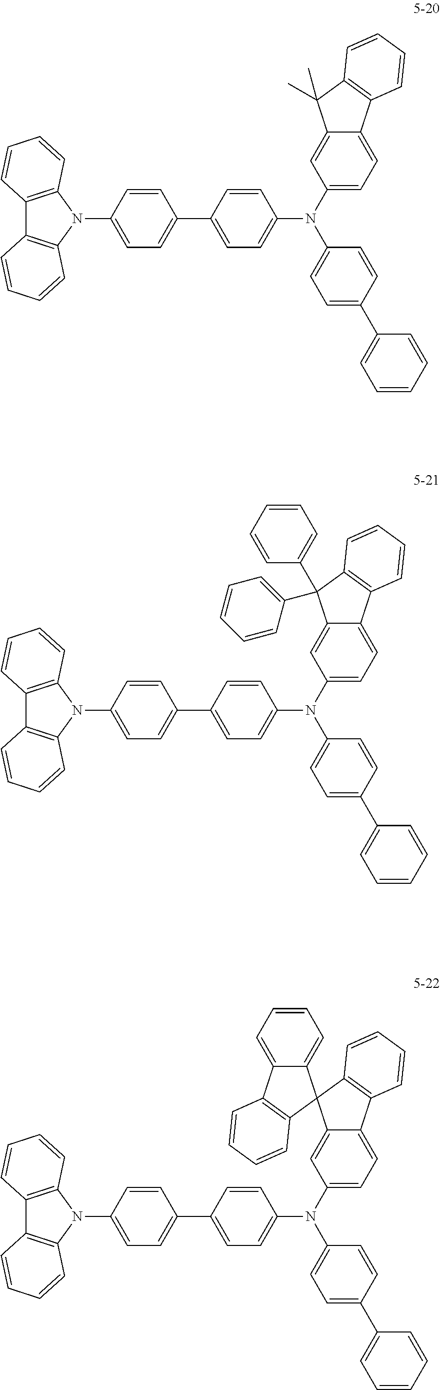 [SMILES: CC1(C)c2ccccc2-c2ccc(N(c3ccc(-c4ccccc4)cc3)c3ccc(-c4ccc(-n5c6ccccc6c6ccccc65)cc4)cc3)cc21 ; c1ccc(-c2ccc(N(c3ccc(-c4ccc(-n5c6ccccc6c6ccccc65)cc4)cc3)c3ccc4c(c3)C(c3ccccc3)(c3ccccc3)c3ccccc3-4)cc2)cc1 ; c1ccc(-c2ccc(N(c3ccc(-c4ccc(-n5c6ccccc6c6ccccc65)cc4)cc3)c3ccc4c(c3)C3(c5ccccc5-c5ccccc53)c3ccccc3-4)cc2)cc1]

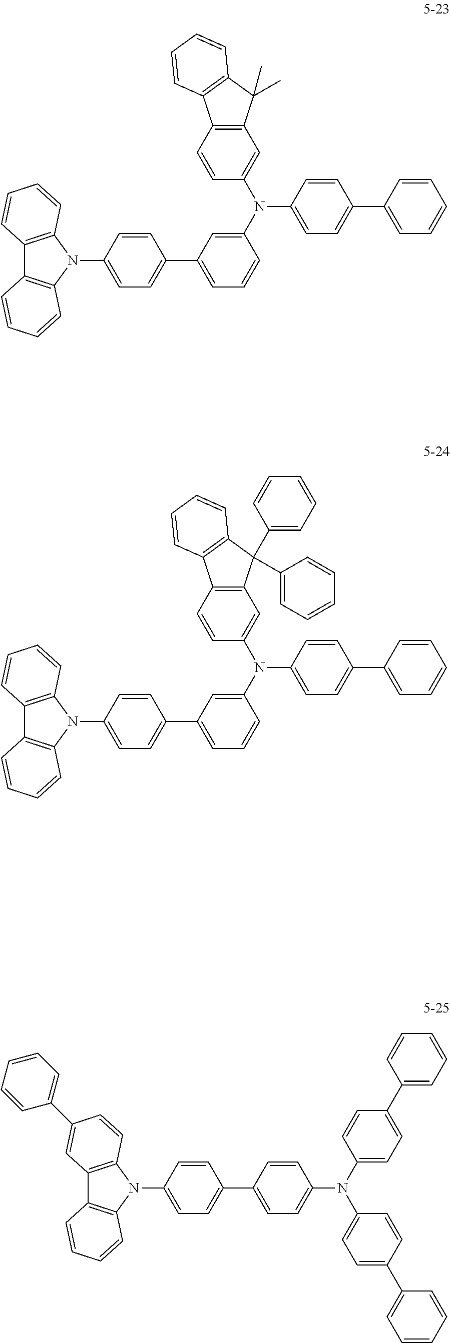 [SMILES: CC1(C)c2ccccc2-c2ccc(N(c3ccc(-c4ccccc4)cc3)c3cccc(-c4ccc(-n5c6ccccc6c6ccccc65)cc4)c3)cc21 ; c1ccc(-c2ccc(N(c3cccc(-c4ccc(-n5c6ccccc6c6ccccc65)cc4)c3)c3ccc4c(c3)C(c3ccccc3)(c3ccccc3)c3ccccc3-4)cc2)cc1 ; c1ccc(-c2ccc(N(c3ccc(-c4ccccc4)cc3)c3ccc(-c4ccc(-n5c6ccccc6c6cc(-c7ccccc7)ccc65)cc4)cc3)cc2)cc1]

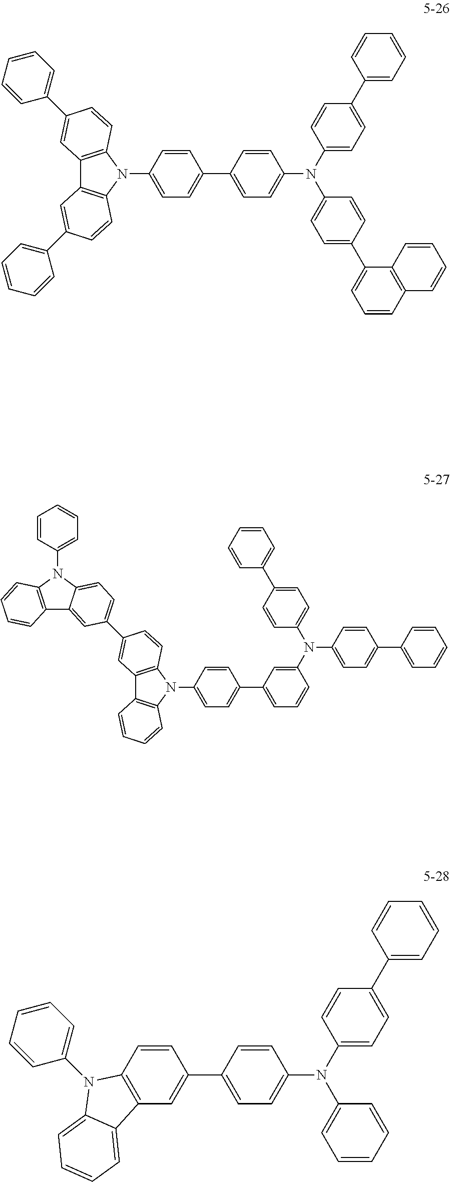 [SMILES: c1ccc(-c2ccc(N(c3ccc(-c4ccc(-n5c6ccc(-c7ccccc7)cc6c6cc(-c7ccccc7)ccc65)cc4)cc3)c3ccc(-c4cccc5ccccc45)cc3)cc2)cc1 ; c1ccc(-c2ccc(N(c3ccc(-c4ccccc4)cc3)c3cccc(-c4ccc(-n5c6ccccc6c6cc(-c7ccc8c(c7)c7ccccc7n8-c7ccccc7)ccc65)cc4)c3)cc2)cc1 ; c1ccc(-c2ccc(N(c3ccccc3)c3ccc(-c4ccc5c(c4)c4ccccc4n5-c4ccccc4)cc3)cc2)cc1]

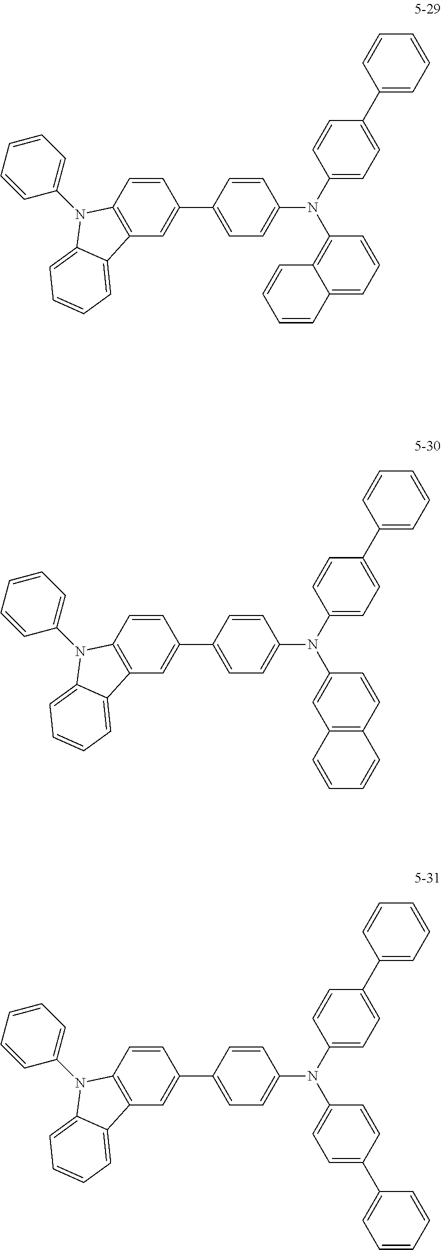 [SMILES: c1ccc(-c2ccc(N(c3ccc(-c4ccc5c(c4)c4ccccc4n5-c4ccccc4)cc3)c3cccc4ccccc34)cc2)cc1 ; c1ccc(-c2ccc(N(c3ccc(-c4ccc5c(c4)c4ccccc4n5-c4ccccc4)cc3)c3ccc4ccccc4c3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1]

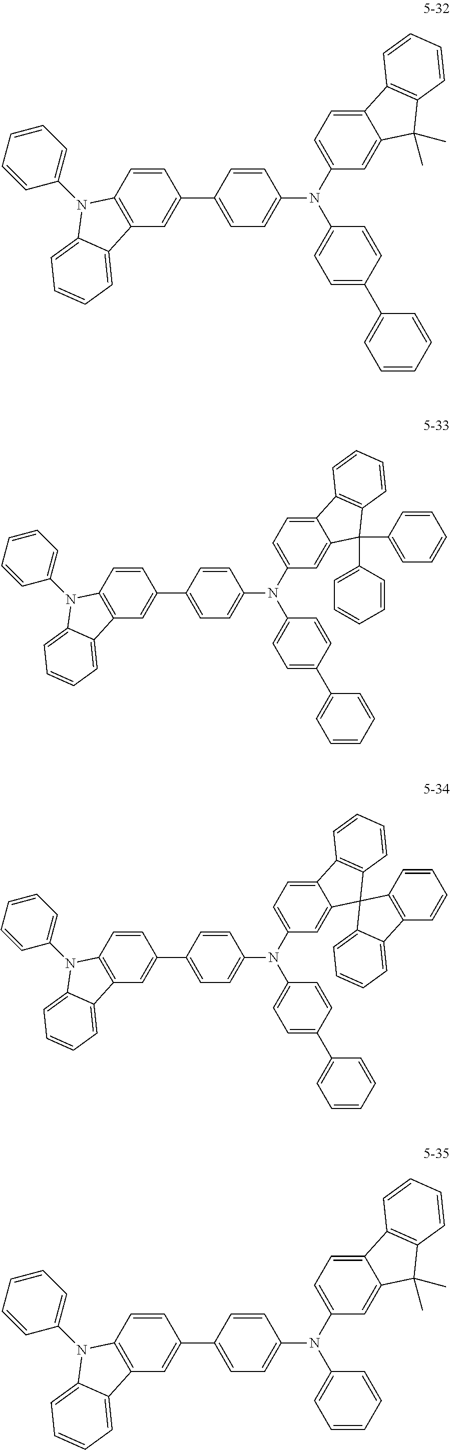 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1 ; CC1(C)c2ccccc2-c2ccc(N(c3ccccc3)c3ccc(-c4ccc5c(c4)c4ccccc4n5-c4ccccc4)cc3)cc21]

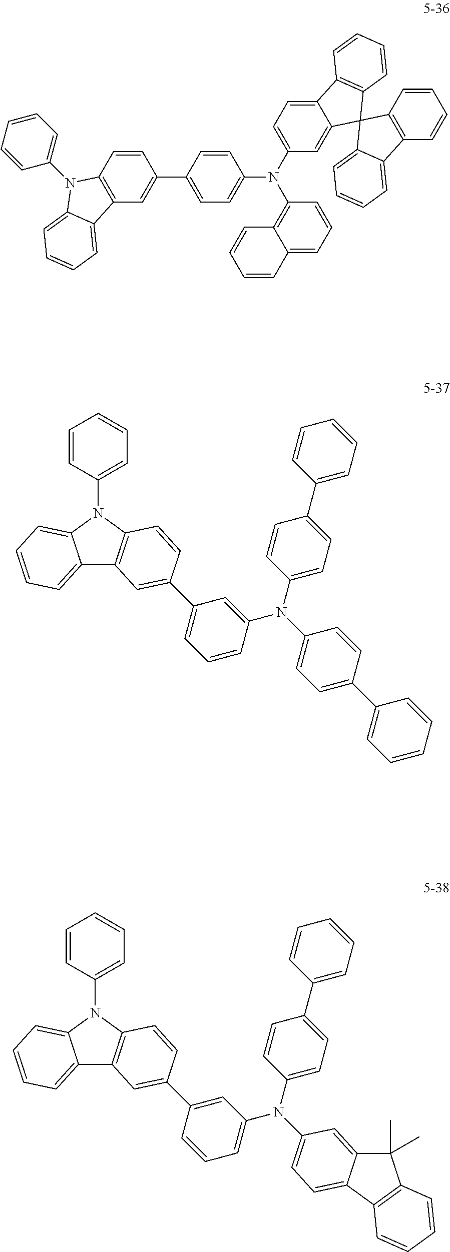 [SMILES: c1ccc(-n2c3ccccc3c3cc(-c4ccc(N(c5ccc6c(c5)C5(c7ccccc7-c7ccccc75)c5ccccc5-6)c5cccc6ccccc56)cc4)ccc32)cc1 ; c1ccc(-c2ccc(N(c3ccc(-c4ccccc4)cc3)c3cccc(-c4ccc5c(c4)c4ccccc4n5-c4ccccc4)c3)cc2)cc1 ; CC1(C)c2ccccc2-c2ccc(N(c3ccc(-c4ccccc4)cc3)c3cccc(-c4ccc5c(c4)c4ccccc4n5-c4ccccc4)c3)cc21]

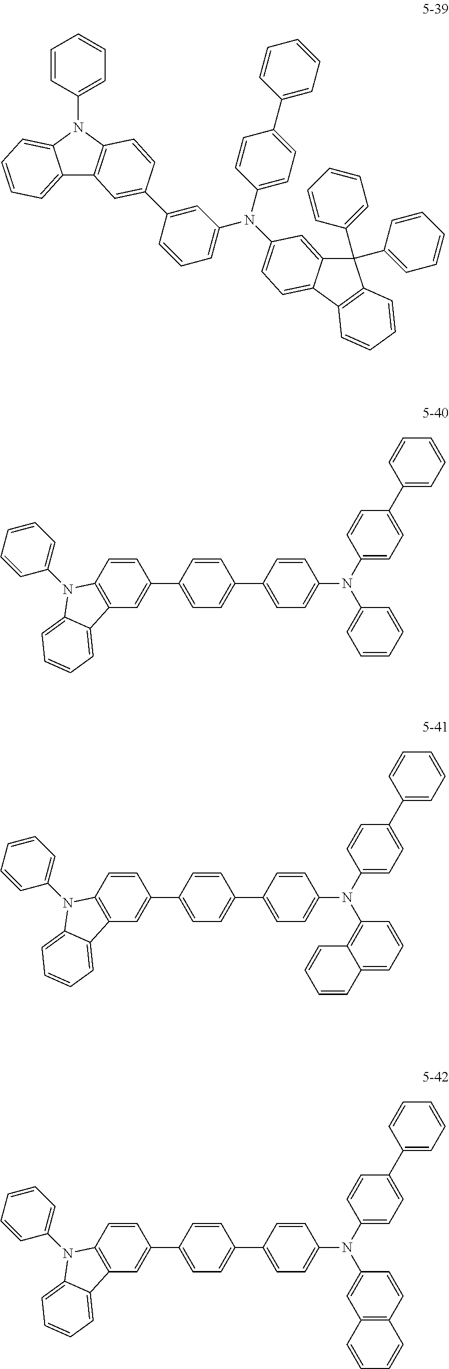 [SMILES: c1ccc(-c2ccc(N(c3cccc(-c4ccc5c(c4)c4ccccc4n5-c4ccccc4)c3)c3ccc4c(c3)C(c3ccccc3)(c3ccccc3)c3ccccc3-4)cc2)cc1 ; c1ccc(-c2ccc(N(c3ccccc3)c3ccc(-c4ccc(-c5ccc6c(c5)c5ccccc5n6-c5ccccc5)cc4)cc3)cc2)cc1 ; c1ccc(-c2ccc(N(c3ccc(-c4ccc(-c5ccc6c(c5)c5ccccc5n6-c5ccccc5)cc4)cc3)c3cccc4ccccc34)cc2)cc1 ; c1ccc(-c2ccc(N(c3ccc(-c4ccc(-c5ccc6c(c5)c5ccccc5n6-c5ccccc5)cc4)cc3)c3ccc4ccccc4c3)cc2)cc1]

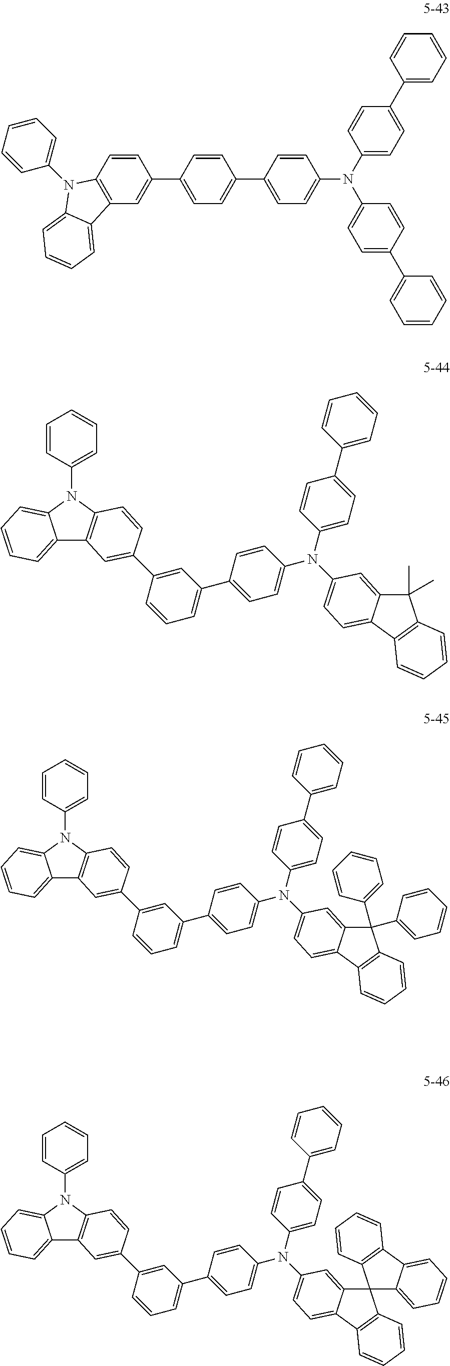 [SMILES: c1ccc(-c2ccc(N(c3ccc(-c4ccccc4)cc3)c3ccc(-c4ccc(-c5ccc6c(c5)c5ccccc5n6-c5ccccc5)cc4)cc3)cc2)cc1 ; CC1(C)c2ccccc2-c2ccc(N(c3ccc(-c4ccccc4)cc3)c3ccc(-c4cccc(-c5ccc6c(c5)c5ccccc5n6-c5ccccc5)c4)cc3)cc21 ; c1ccc(-c2ccc(N(c3ccc(-c4cccc(-c5ccc6c(c5)c5ccccc5n6-c5ccccc5)c4)cc3)c3ccc4c(c3)C(c3ccccc3)(c3ccccc3)c3ccccc3-4)cc2)cc1 ; c1ccc(-c2ccc(N(c3ccc(-c4cccc(-c5ccc6c(c5)c5ccccc5n6-c5ccccc5)c4)cc3)c3ccc4c(c3)C3(c5ccccc5-c5ccccc53)c3ccccc3-4)cc2)cc1]

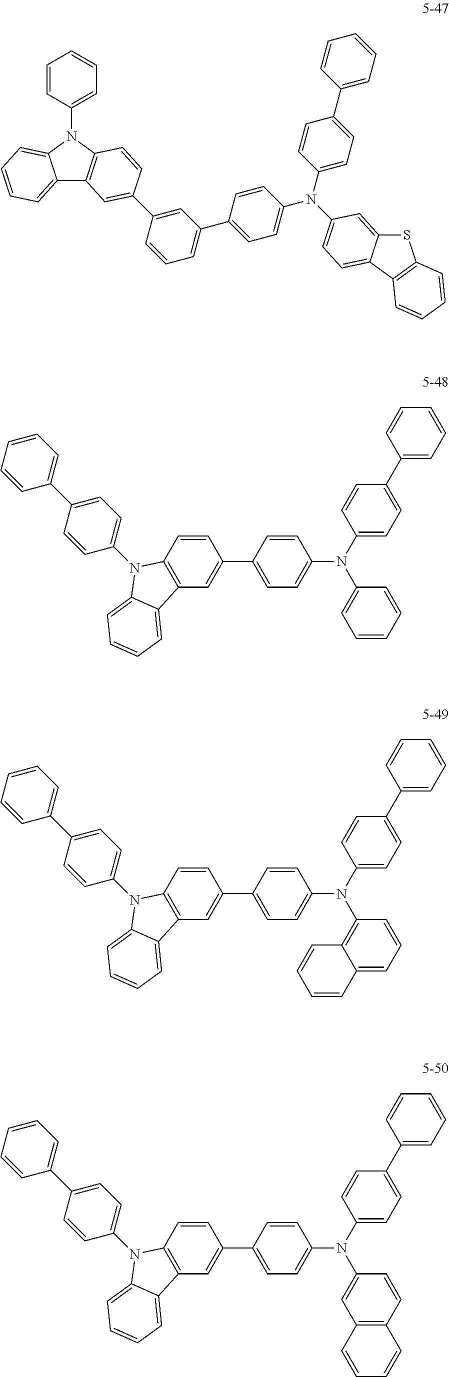 [SMILES: c1ccc(-c2ccc(N(c3ccc(-c4cccc(-c5ccc6c(c5)c5ccccc5n6-c5ccccc5)c4)cc3)c3ccc4c(c3)sc3ccccc34)cc2)cc1 ; c1ccc(-c2ccc(N(c3ccccc3)c3ccc(-c4ccc5c(c4)c4ccccc4n5-c4ccc(-c5ccccc5)cc4)cc3)cc2)cc1 ; c1ccc(-c2ccc(N(c3ccc(-c4ccc5c(c4)c4ccccc4n5-c4ccc(-c5ccccc5)cc4)cc3)c3cccc4ccccc34)cc2)cc1 ; c1ccc(-c2ccc(N(c3ccc(-c4ccc5c(c4)c4ccccc4n5-c4ccc(-c5ccccc5)cc4)cc3)c3ccc4ccccc4c3)cc2)cc1]

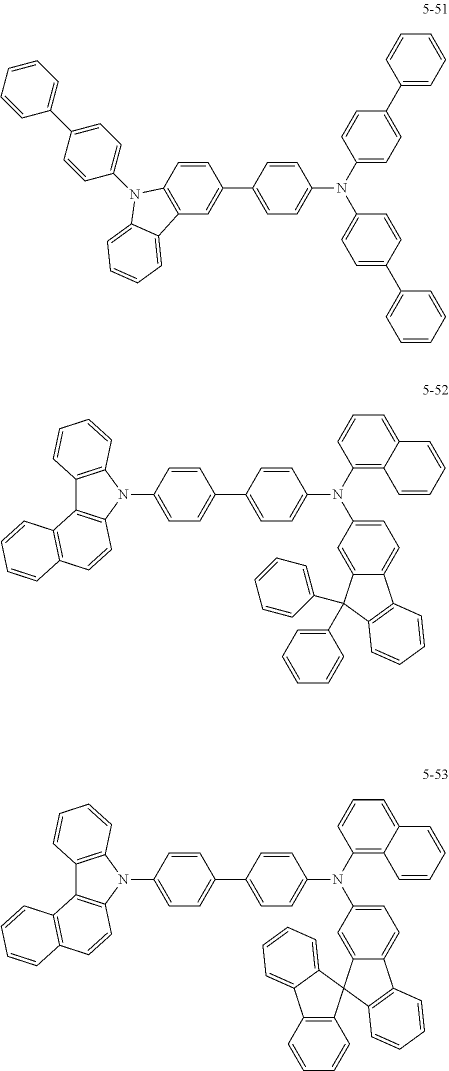 [SMILES: c1ccc(-c2ccc(N(c3ccc(-c4ccccc4)cc3)c3ccc(-c4ccc5c(c4)c4ccccc4n5-c4ccc(-c5ccccc5)cc4)cc3)cc2)cc1 ; c1ccc(C2(c3ccccc3)c3ccccc3-c3ccc(N(c4ccc(-c5ccc(-n6c7ccccc7c7c8ccccc8ccc76)cc5)cc4)c4cccc5ccccc45)cc32)cc1 ; c1ccc2c(c1)-c1ccccc1C21c2ccccc2-c2ccc(N(c3ccc(-c4ccc(-n5c6ccccc6c6c7ccccc7ccc65)cc4)cc3)c3cccc4ccccc34)cc21]

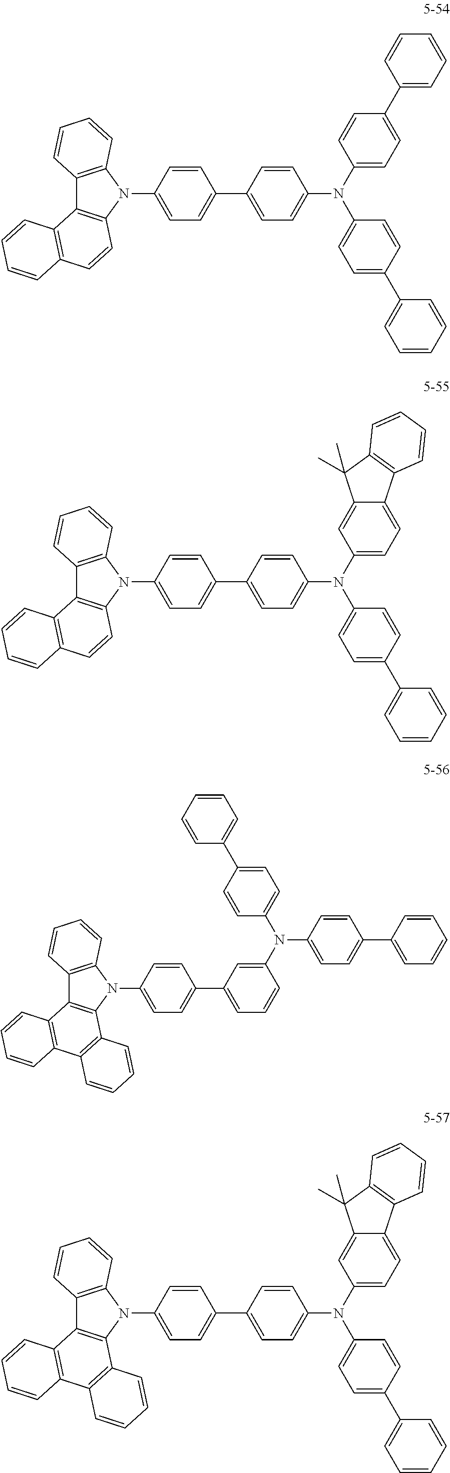 [SMILES: c1ccc(-c2ccc(N(c3ccc(-c4ccccc4)cc3)c3ccc(-c4ccc(-n5c6ccccc6c6c7ccccc7ccc65)cc4)cc3)cc2)cc1 ; CC1(C)c2ccccc2-c2ccc(N(c3ccc(-c4ccccc4)cc3)c3ccc(-c4ccc(-n5c6ccccc6c6c7ccccc7ccc65)cc4)cc3)cc21 ; c1ccc(-c2ccc(N(c3ccc(-c4ccccc4)cc3)c3cccc(-c4ccc(-n5c6ccccc6c6c7ccccc7c7ccccc7c65)cc4)c3)cc2)cc1 ; CC1(C)c2ccccc2-c2ccc(N(c3ccc(-c4ccccc4)cc3)c3ccc(-c4ccc(-n5c6ccccc6c6c7ccccc7c7ccccc7c65)cc4)cc3)cc21]

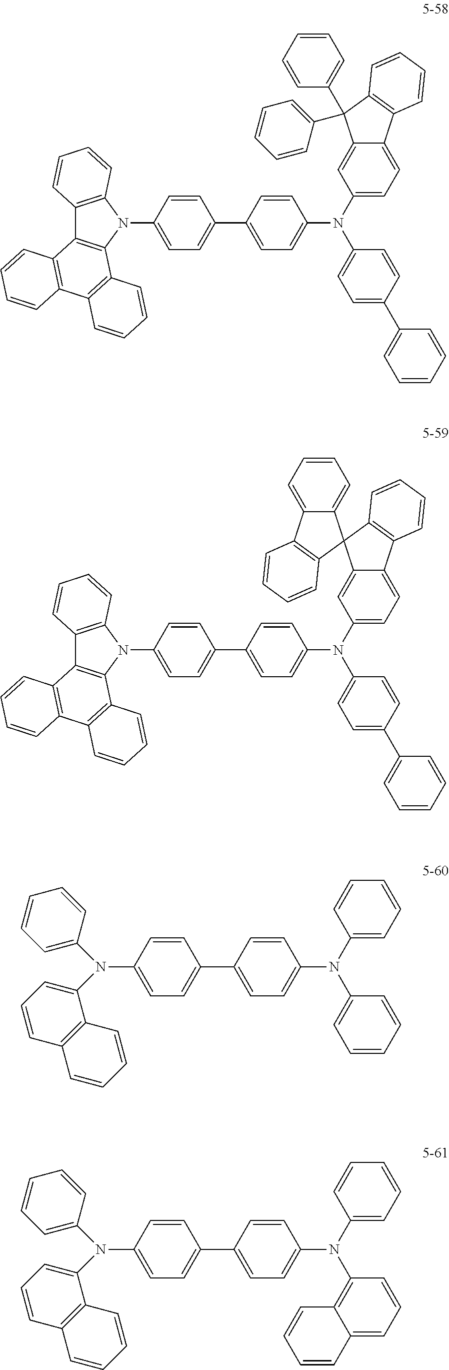 [SMILES: c1ccc(-c2ccc(N(c3ccc(-c4ccc(-n5c6ccccc6c6c7ccccc7c7ccccc7c65)cc4)cc3)c3ccc4c(c3)C(c3ccccc3)(c3ccccc3)c3ccccc3-4)cc2)cc1 ; c1ccc(-c2ccc(N(c3ccc(-c4ccc(-n5c6ccccc6c6c7ccccc7c7ccccc7c65)cc4)cc3)c3ccc4c(c3)C3(c5ccccc5-c5ccccc53)c3ccccc3-4)cc2)cc1 ; c1ccc(N(c2ccccc2)c2ccc(-c3ccc(N(c4ccccc4)c4cccc5ccccc45)cc3)cc2)cc1 ; c1ccc(N(c2ccc(-c3ccc(N(c4ccccc4)c4cccc5ccccc45)cc3)cc2)c2cccc3ccccc23)cc1]

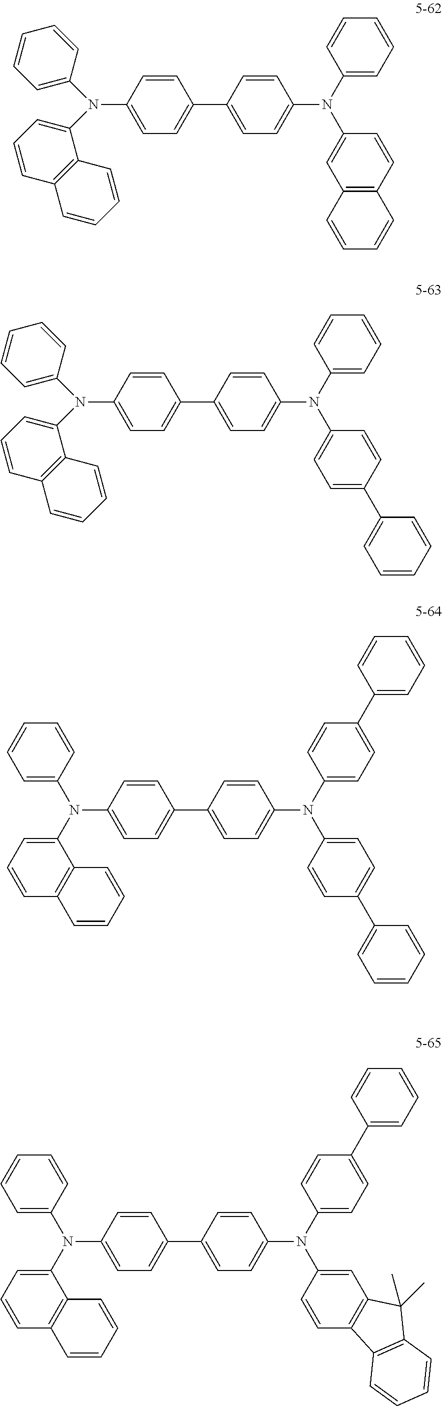 [SMILES: c1ccc(N(c2ccc(-c3ccc(N(c4ccccc4)c4cccc5ccccc45)cc3)cc2)c2ccc3ccccc3c2)cc1 ; c1ccc(-c2ccc(N(c3ccccc3)c3ccc(-c4ccc(N(c5ccccc5)c5cccc6ccccc56)cc4)cc3)cc2)cc1 ; c1ccc(-c2ccc(N(c3ccc(-c4ccccc4)cc3)c3ccc(-c4ccc(N(c5ccccc5)c5cccc6ccccc56)cc4)cc3)cc2)cc1 ; CC1(C)c2ccccc2-c2ccc(N(c3ccc(-c4ccccc4)cc3)c3ccc(-c4ccc(N(c5ccccc5)c5cccc6ccccc56)cc4)cc3)cc21]

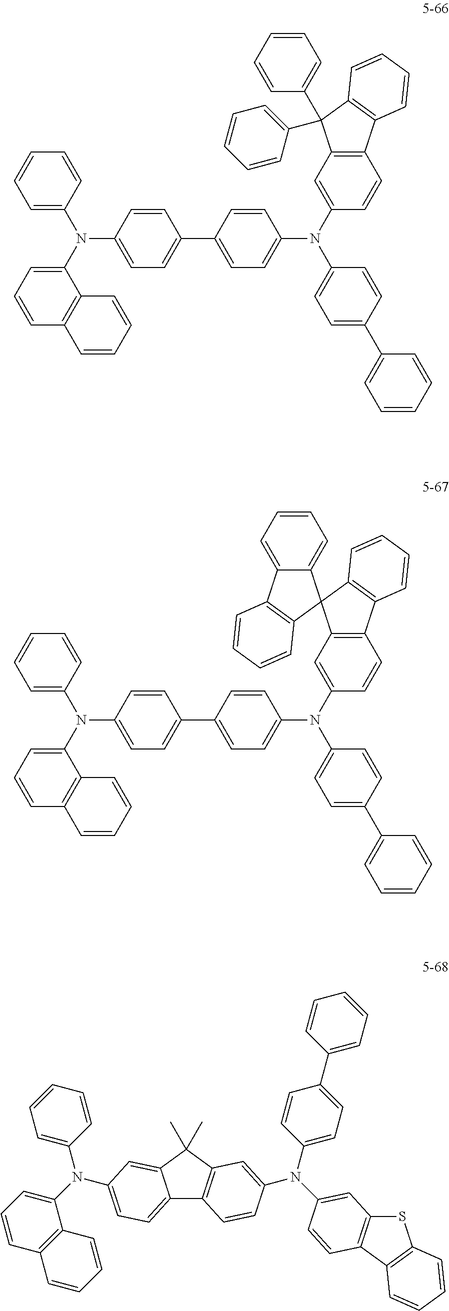 [SMILES: c1ccc(-c2ccc(N(c3ccc(-c4ccc(N(c5ccccc5)c5cccc6ccccc56)cc4)cc3)c3ccc4c(c3)C(c3ccccc3)(c3ccccc3)c3ccccc3-4)cc2)cc1 ; c1ccc(-c2ccc(N(c3ccc(-c4ccc(N(c5ccccc5)c5cccc6ccccc56)cc4)cc3)c3ccc4c(c3)C3(c5ccccc5-c5ccccc53)c3ccccc3-4)cc2)cc1 ; CC1(C)c2cc(N(c3ccc(-c4ccccc4)cc3)c3ccc4c(c3)sc3ccccc34)ccc2-c2ccc(N(c3ccccc3)c3cccc4ccccc34)cc21]

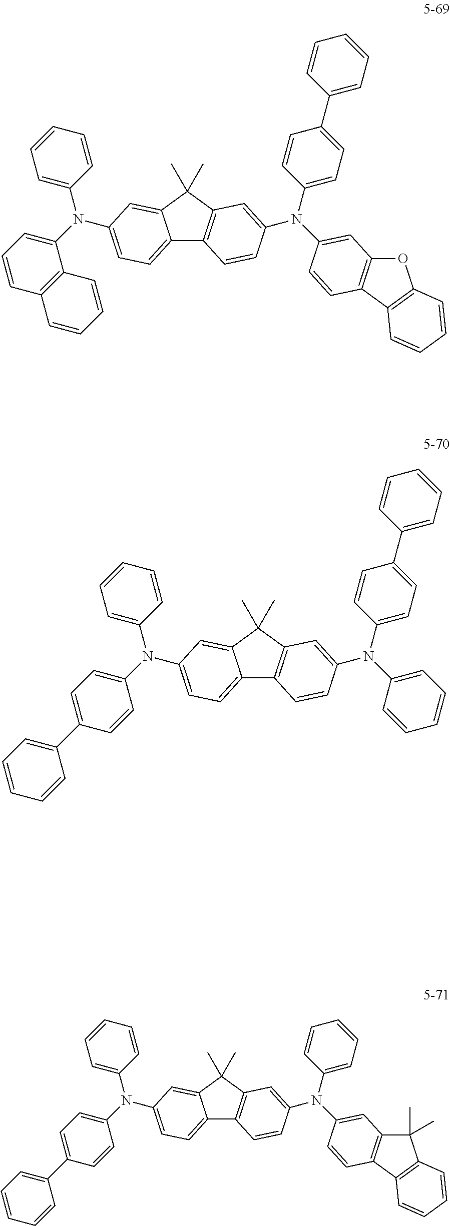 [SMILES: CC1(C)c2cc(N(c3ccc(-c4ccccc4)cc3)c3ccc4c(c3)oc3ccccc34)ccc2-c2ccc(N(c3ccccc3)c3cccc4ccccc34)cc21 ; CC1(C)c2cc(N(c3ccccc3)c3ccc(-c4ccccc4)cc3)ccc2-c2ccc(N(c3ccccc3)c3ccc(-c4ccccc4)cc3)cc21 ; CC1(C)c2ccccc2-c2ccc(N(c3ccccc3)c3ccc4c(c3)C(C)(C)c3cc(N(c5ccccc5)c5ccc(-c6ccccc6)cc5)ccc3-4)cc21]

Referring to the Figure, the organic electric element(100) according to the present invention comprises a first electrode(120) formed on a substrate(110), a second electrode(180), and an organic material layer including the compound represented by Formula (1) between the first electrode(120) and the second electrode(180). Here, the first electrode(120) may be an anode (positive electrode), and the second electrode(180) may be a cathode (negative electrode). In the case of an inverted organic electric element, the first electrode may be a cathode, and the second electrode may be an anode.

The organic material layer may comprise a hole injection layer(130), a hole transport layer(140), an emitting layer(150), an emitting auxiliary layer(151), an electron transport layer(160), and an electron injection layer(170) formed in sequence on the first electrode(120).

Although not shown, the organic electric element according to the present invention may further comprise a protective layer formed on at least one surface of the first electrode and the second electrode opposite to the organic material layer.

Otherwise, even if the same core is used, the band gap, the electrical characteristics, the interface characteristics, and the like may vary depending on which substituent is bonded at which position, therefore the choice of core and the combination of sub-substituents associated therewith is also very important, and in particular, when the optimal combination of energy levels, T1 values and unique properties of materials(mobility, interfacial characteristics, etc.) of each organic material layer is achieved, a long life span and high efficiency can be achieved at the same time.

The organic electroluminescent device according to an embodiment of the present invention may be manufactured using a PVD (physical vapor deposition) method. For example, a metal or a metal oxide having conductivity or an alloy thereof is deposited on a substrate to form a cathode, and the organic material layer including the hole injection layer(130), the hole transport layer(140), the emitting layer(150), the emitting auxiliary layer(151), the electron transport layer(160), and the electron injection layer(170) is formed thereon, and then depositing a material usable as a cathode thereon can manufacture an organic electroluminescent device according to an embodiment of the present invention.

Accordingly, the present invention provides an organic electric element comprising a first electrode; a second electrode; and an organic material layer between the first electrode and the second electrode, wherein the organic material layer comprises a hole injection layer, a hole transport layer, an emitting auxiliary layer and an emitting layer, and wherein the organic material layer comprises a compound included in Formula (1).

In addition, the present invention provides a compound, wherein at least one of the hole injection layer, the hole transport layer, the emitting-auxiliary layer, and the emitting layer includes a compound according to Formula (1), wherein the compound comprises one compound or at least 2 or more compounds.

Also, the present invention provides an organic electric element wherein the emitting auxiliary layer contains a single compound or at least 2 or more compounds of the above compounds.

The present invention provides a compound further comprising a light efficiency enhancing layer formed on at least one of the opposite side to the organic material layer among one side of the first electrode, or one of the opposite side to the organic material layer among one side of the second electrode.

Also, the present invention provides the organic electric element wherein the organic material layer is formed by one of a spin coating process, a nozzle printing process, an inkjet printing process, a slot coating process, a dip coating process or a roll-to-roll process, and since the organic material layer according to the present invention can be formed by various methods, the scope of the present invention is not limited by the method of forming the organic material layer.

As another specific example, the present invention provides an organic electric element characterized in that the organic layer is mixed with the same or different compound of the compound represented by Formula (1).

The present invention also provides an organic electric element wherein a hole transport layer and an emitting-auxiliary layer contain a compound represented by Formula (1), and wherein a hole transport layer or an emitting-auxiliary layer contains a compound represented by Formula (1) in another aspect.

The present invention also provides an electronic device comprising a display device including the organic electric element; and a control unit for driving the display device.

According to another aspect, the present invention provides an electronic device characterized in that the organic electric element is at least one of an OLED, an organic solar cell, an organic photo conductor, an organic transistor and an element for monochromic or white illumination. Here, the electronic device may be a wired/wireless communication terminal which is currently used or will be used in the future, and covers all kinds of electronic devices including a mobile communication terminal such as a cellular phone, a personal digital assistant(PDA), an electronic dictionary, a point-to-multipoint(PMP), a remote controller, a navigation unit, a game player, various kinds of TVs, and various kinds of computers.

Hereinafter, synthesis examples of the compound represented by Formula (1) according to the present invention and preparation examples of the organic electric element will be described in detail by way of example, but are not limited to the following examples of the invention.

### SYNTHESIS EXAMPLE 1

The final products 1 represented by Formula (1) of the present invention can be synthesized by reaction between Sub 1 and Sub 2 as illustrated in the following Reaction Scheme 1.

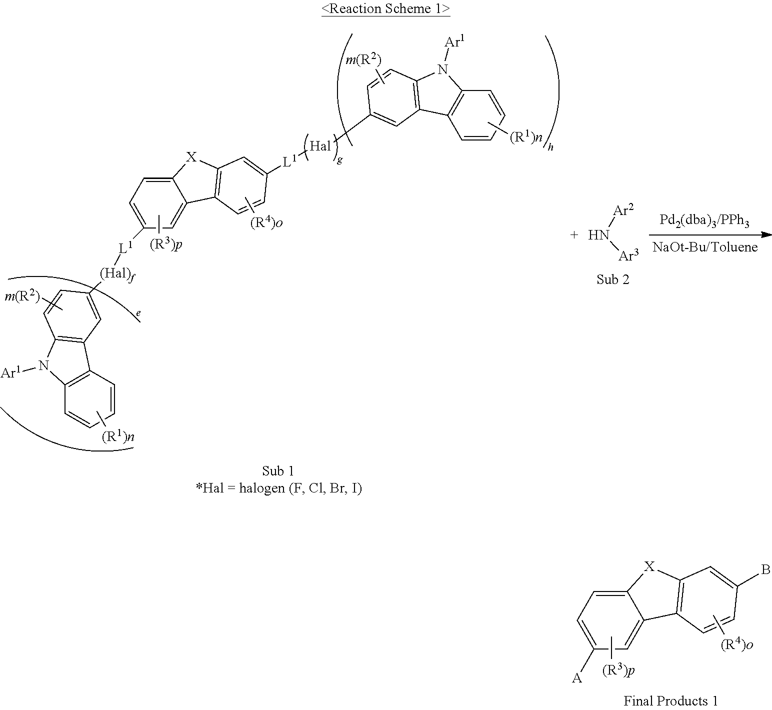

(1) When e is 1, f and h are 0 and g is 1.

(2) When h is 1, g and e are 0 and f is 1.

Synthesis Examples of Sub 1

Sub 1 of Reaction Scheme 1 can be synthesized by the reaction path of the following Reaction Scheme 2, but is not limited thereto.

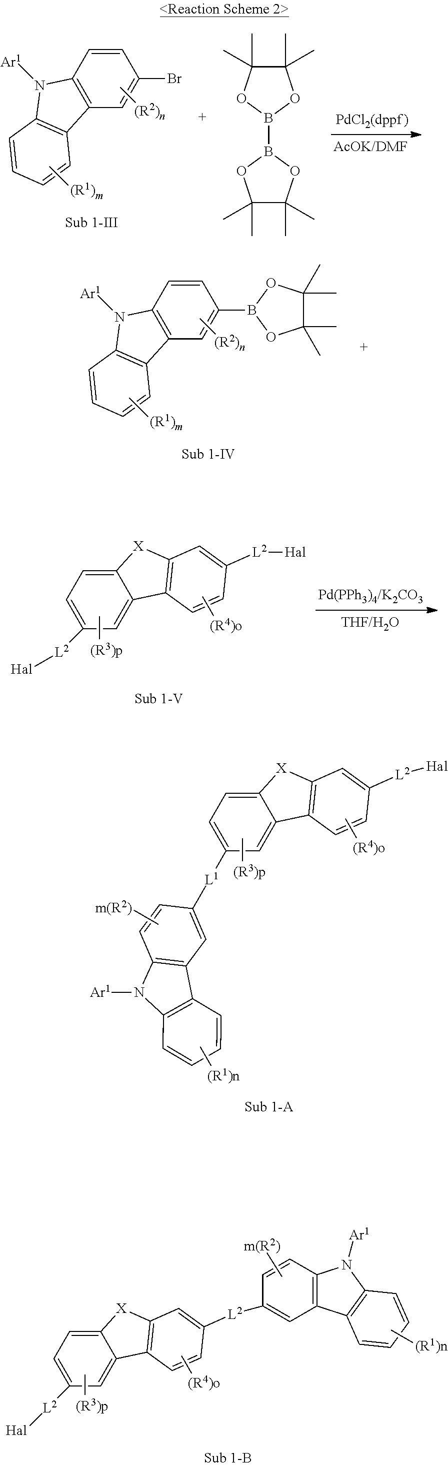

Synthesis Examples of Sub 1-I

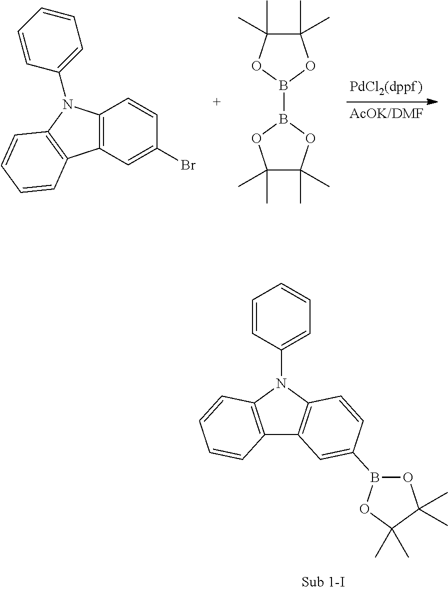

In a round bottom flask, 3-bromo-9-phenyl-9H-carbazole (76.78 g, 238.3 mmol) was dissolved in DMF and Bis(pinacolato)diboron (66.57 g, 262.1 mmol), Pd(dppf)Cl₂ (5.84 g, 7.1 mmol), KOAc (70.16 g, 714.9 mmol) were added and stirred at 90° C. When the reaction was completed, the DMF is removed by distillation and extracted with CH₂Cl₂ and water. The organic layer was dried over MgSO₄ and concentrated. The resulting compound was separated by silicagel column chromatography and recrystallized to obtain 73.92 g (yield: 84%) of the product.

Synthesis Examples of Sub 1 1)

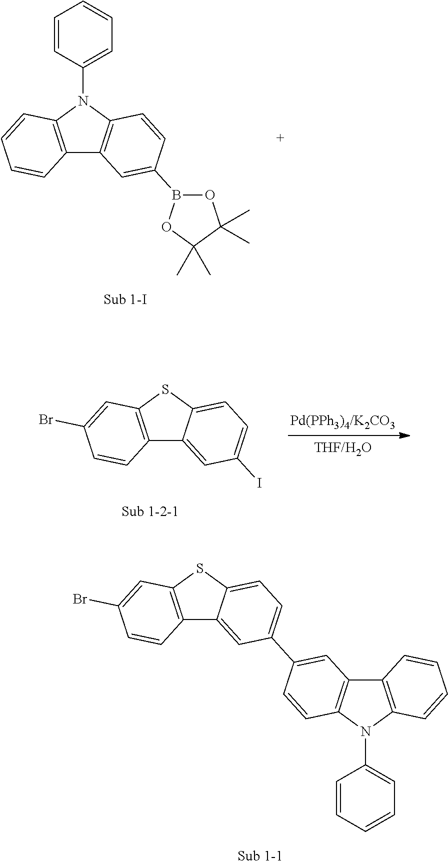 [SMILES: CC1(C)OB(c2ccc3c(c2)c2ccccc2n3-c2ccccc2)OC1(C)C ; Brc1ccc2c(c1)sc1ccc(I)cc12 ; Brc1ccc2c(c1)sc1ccc(-c3ccc4c(c3)c3ccccc3n4-c3ccccc3)cc12]

Sub 1-I (73.92 g, 200.2 mmol) was dissolved in THF 880 ml in a round bottom flask, and Sub 1-2-1 (116.8 g, 300.3 mmol), Pd(PPh₃)₄ (11.6 g, 10 mmol), K₂CO₃ (83 g, 600.6 mmol) and water (440 mL) were added and stirred at 80° C. When the reaction was completed, the reaction mixture was extracted with CH₂Cl₂ and water. The organic layer was dried over MgSO₄ and concentrated. The resulting compound was separated by silicagel column chromatography and recrystallized to obtain 81.8 g (yield: 81%) of the product.

Synthesis Examples of Sub 1 2)

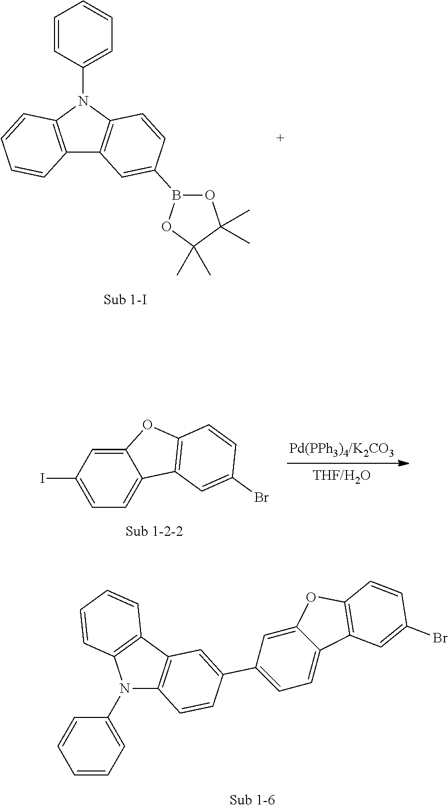 [SMILES: CC1(C)OB(c2ccc3c(c2)c2ccccc2n3-c2ccccc2)OC1(C)C ; Brc1ccc2oc3cc(I)ccc3c2c1 ; Brc1ccc2oc3cc(-c4ccc5c(c4)c4ccccc4n5-c4ccccc4)ccc3c2c1]

Sub 1-I (73.92 g, 200.2 mmol) and Sub 1-2-2 (112.0 g, 300.3 mmol) were reacted using the synthesis method of Sub 1-1 described above to give 76.3 g of the product. (yield: 78%).

Synthesis Examples of Sub 1 3)

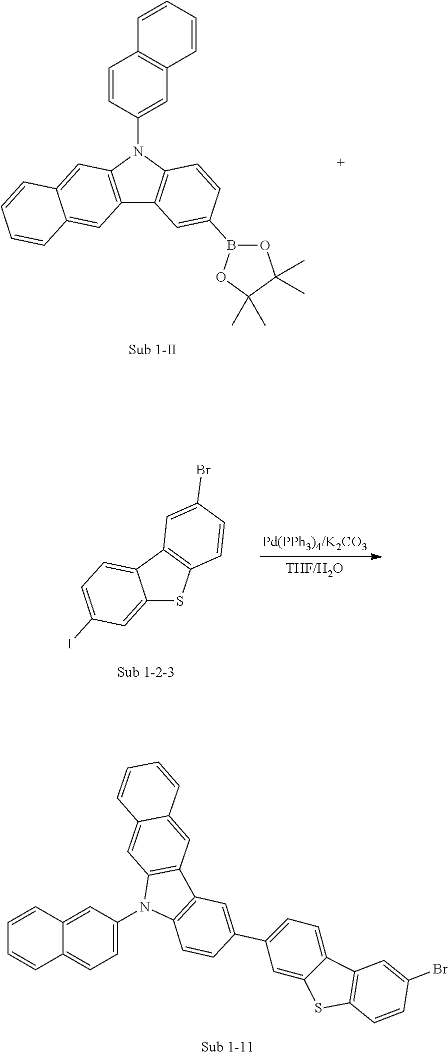 [SMILES: CC1(C)OB(c2ccc3c(c2)c2cc4ccccc4cc2n3-c2ccc3ccccc3c2)OC1(C)C ; Brc1ccc2sc3cc(I)ccc3c2c1 ; Brc1ccc2sc3cc(-c4ccc5c(c4)c4cc6ccccc6cc4n5-c4ccc5ccccc5c4)ccc3c2c1]

Sub 1-II (93.9 g, 200.2 mmol) and Sub 1-2-3 (116.8 g, 300.3 mmol) were reacted using the synthesis method of Sub 1-1 described above to give 90.8 g of the product. (yield: 75%).

Synthesis Examples of Sub 1 4)

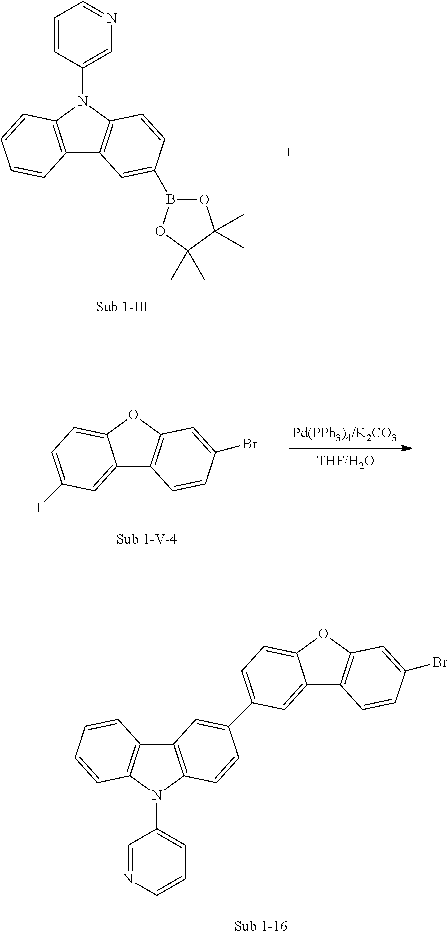 [SMILES: CC1(C)OB(c2ccc3c(c2)c2ccccc2n3-c2cccnc2)OC1(C)C ; Brc1ccc2c(c1)oc1ccc(I)cc12 ; Brc1ccc2c(c1)oc1ccc(-c3ccc4c(c3)c3ccccc3n4-c3cccnc3)cc12]

Sub 1-III (74.1 g, 200.2 mmol) and Sub 1-2-4 (112.0 g, 300.3 mmol) were reacted using the synthesis method of Sub 1-1 described above to give 70.5 g of the product. (yield: 72%).

Examples of Sub 1 include, but are not limited to, the following.

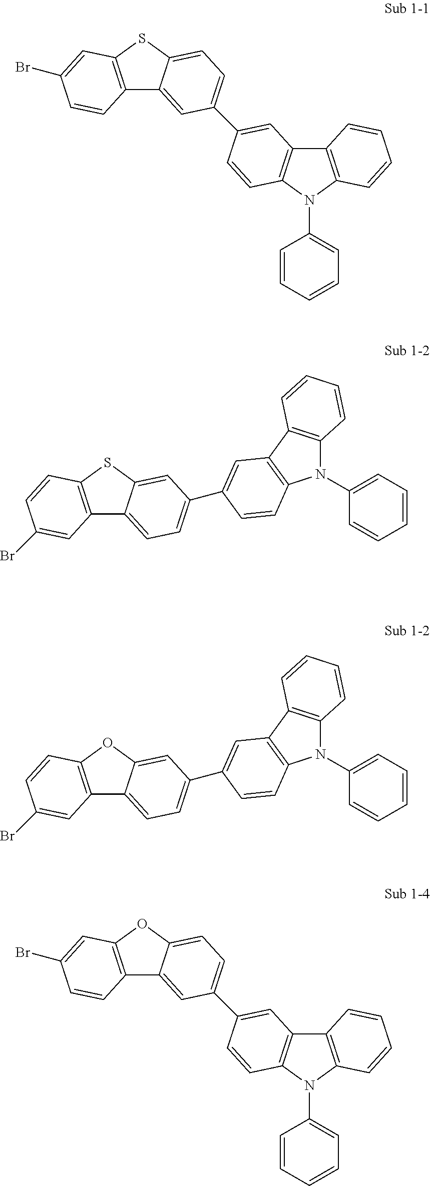 [SMILES: Brc1ccc2c(c1)sc1ccc(-c3ccc4c(c3)c3ccccc3n4-c3ccccc3)cc12 ; Brc1ccc2sc3cc(-c4ccc5c(c4)c4ccccc4n5-c4ccccc4)ccc3c2c1 ; Brc1ccc2oc3cc(-c4ccc5c(c4)c4ccccc4n5-c4ccccc4)ccc3c2c1 ; Brc1ccc2c(c1)oc1ccc(-c3ccc4c(c3)c3ccccc3n4-c3ccccc3)cc12]

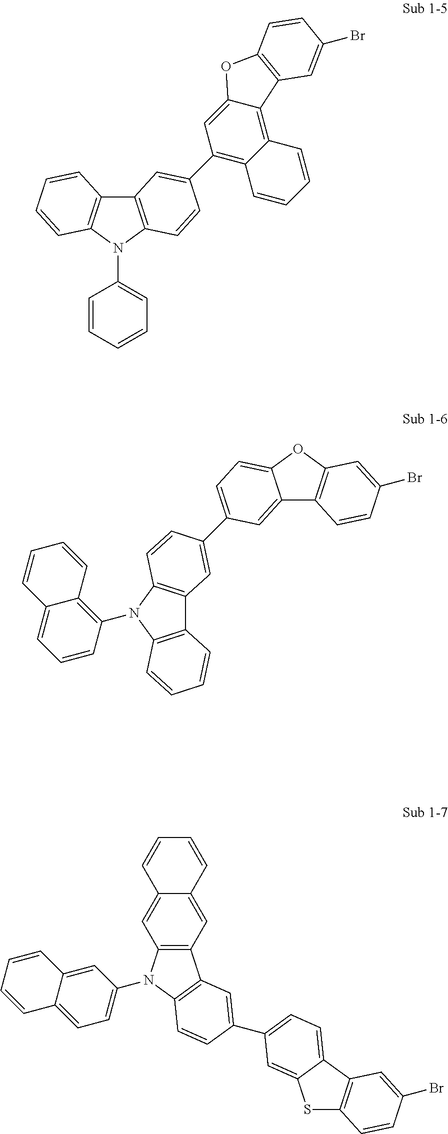 [SMILES: Brc1ccc2oc3cc(-c4ccc5c(c4)c4ccccc4n5-c4ccccc4)c4ccccc4c3c2c1 ; Brc1ccc2c(c1)oc1ccc(-c3ccc4c(c3)c3ccccc3n4-c3cccc4ccccc34)cc12 ; Brc1ccc2sc3cc(-c4ccc5c(c4)c4cc6ccccc6cc4n5-c4ccc5ccccc5c4)ccc3c2c1]

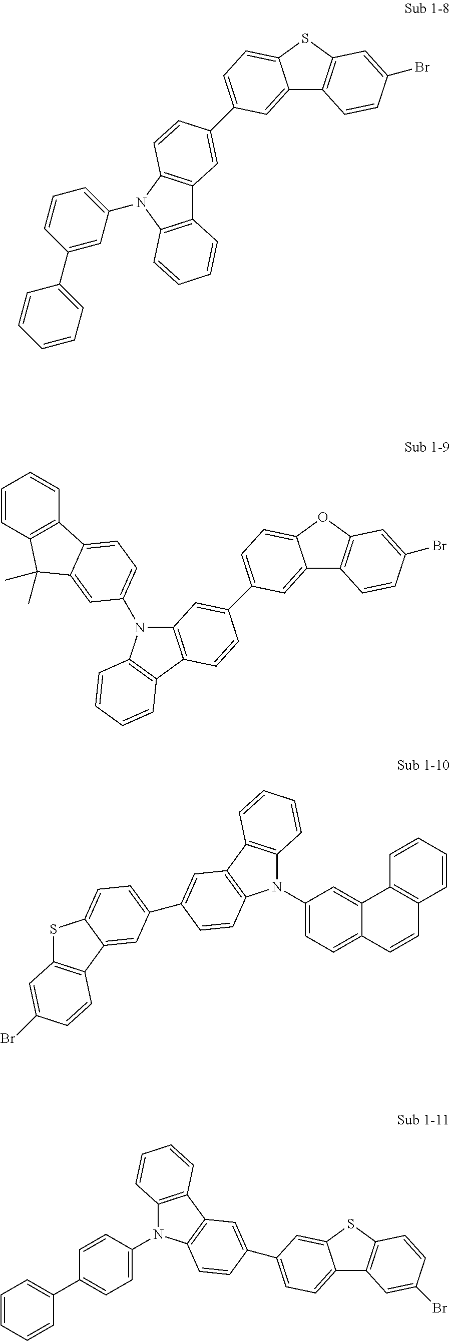 [SMILES: Brc1ccc2c(c1)sc1ccc(-c3ccc4c(c3)c3ccccc3n4-c3cccc(-c4ccccc4)c3)cc12 ; CC1(C)c2ccccc2-c2ccc(-n3c4ccccc4c4ccc(-c5ccc6oc7cc(Br)ccc7c6c5)cc43)cc21 ; Brc1ccc2c(c1)sc1ccc(-c3ccc4c(c3)c3ccccc3n4-c3ccc4ccc5ccccc5c4c3)cc12 ; Brc1ccc2sc3cc(-c4ccc5c(c4)c4ccccc4n5-c4ccc(-c5ccccc5)cc4)ccc3c2c1]

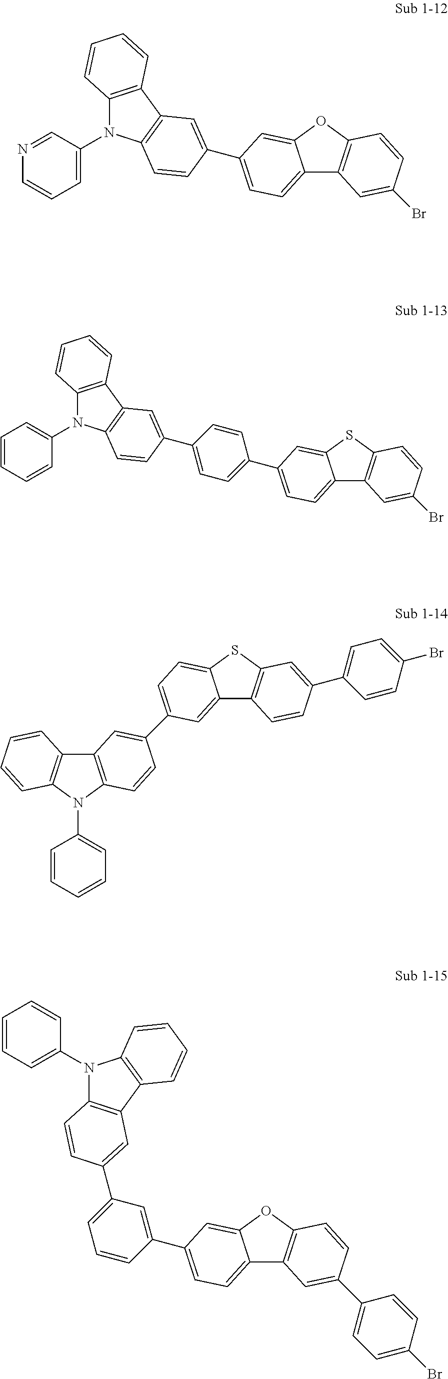 [SMILES: Brc1ccc2oc3cc(-c4ccc5c(c4)c4ccccc4n5-c4cccnc4)ccc3c2c1 ; Brc1ccc2sc3cc(-c4ccc(-c5ccc6c(c5)c5ccccc5n6-c5ccccc5)cc4)ccc3c2c1 ; Brc1ccc(-c2ccc3c(c2)sc2ccc(-c4ccc5c(c4)c4ccccc4n5-c4ccccc4)cc23)cc1 ; Brc1ccc(-c2ccc3oc4cc(-c5cccc(-c6ccc7c(c6)c6ccccc6n7-c6ccccc6)c5)ccc4c3c2)cc1]

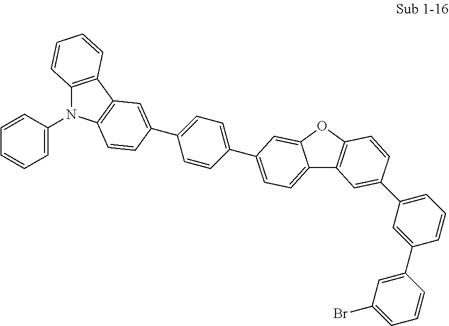 [SMILES: Brc1cccc(-c2cccc(-c3ccc4oc5cc(-c6ccc(-c7ccc8c(c7)c7ccccc7n8-c7ccccc7)cc6)ccc5c4c3)c2)c1]

TABLE 1

| compounds | FD-MS | compounds | FD-MS |
|---|---|---|---|
| Sub 1-1 | m/z = 503.03(C₃₀H₁₈BrNS = 504.44) | Sub 1-2 | m/z = 503.03(C₃₀H₁₈BrNS = 504.44) |
| Sub 1-3 | m/z = 487.06(C₃₀H₁₈BrNO = 488.37) | Sub 1-4 | m/z = 487.06(C₃₀H₁₈BrNO = 488.37) |
| Sub 1-5 | m/z = 537.07(C₃₄H₂₀BrNO = 538.44) | Sub 1-6 | m/z = 537.07(C₃₄H₂₀BrNO = 538.43) |
| Sub 1-7 | m/z = 603.07(C₃₈H₂₂BrNS = 604.56) | Sub 1-8 | m/z = 579.07(C₃₆H₂₂BrNS = 580.54) |
| Sub 1-9 | m/z = 603.12(C₃₉H₂₆BrNO = 604.53) | Sub 1-10 | m/z = 603.07(C₃₈H₂₂BrNS = 604.56) |
| Sub 1-11 | m/z = 579.07(C₃₆H₂₂BrNS = 580.54) | Sub 1-12 | m/z = 488.05(C₂₉H₁₇BrN₂O = 489.36) |
| Sub 1-13 | m/z = 579.07(C₃₆H₂₂BrNS = 580.54) | Sub 1-14 | m/z = 579.07(C₃₆H₂₂BrNS = 580.54) |
| Sub 1-15 | m/z = 639.12(C₄₂H₂₆BrNO = 640.57) | Sub 1-16 | m/z = 715.15(C₄₈H₃₀BrNO = 716.66) |

Synthesis Examples of Sub 2

Sub 2 of Reaction Scheme 1 can be synthesized by the reaction path of the following Reaction Scheme 3, but is not limited thereto.

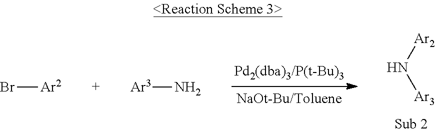

Synthesis Examples of Sub 2-1

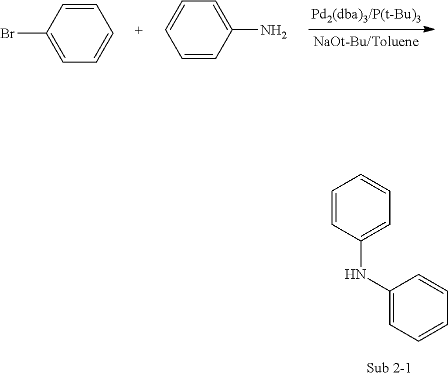 [SMILES: Brc1ccccc1 ; Nc1ccccc1 ; c1ccc(Nc2ccccc2)cc1]

bromobenzene (37.1 g, 236.2 mmol) was added to a round bottom flask and dissolved in toluene (2200 mL), and aniline (20 g, 214.8 mmol), Pd₂(dba)₃ (9.83 g, 10.7 mmol), P(t-Bu)₃ (4.34 g, 21.5 mmol), NaOt-Bu (62 g, 644.3 mmol) were added in order and stirred at 100° C. After the reaction was completed, the reaction mixture was extracted with ether and water. The organic layer was dried over MgSO₄ and concentrated. The resulting compound was separated by silicagel column chromatography and recrystallized to obtain 28 g of the product. (yield: 77%)

Synthesis Examples of Sub 2-13

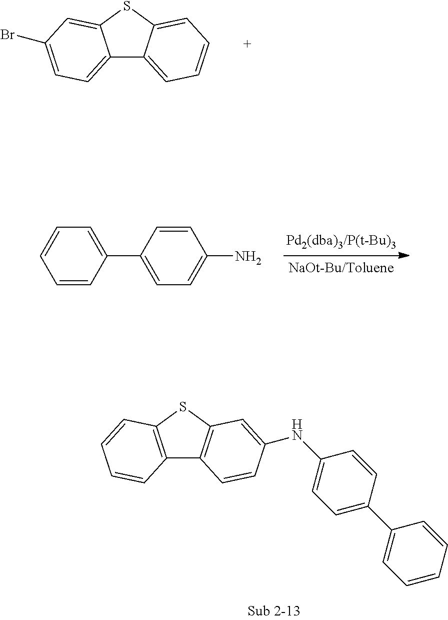 [SMILES: Brc1ccc2c(c1)sc1ccccc12 ; Nc1ccc(-c2ccccc2)cc1 ; c1ccc(-c2ccc(Nc3ccc4c(c3)sc3ccccc34)cc2)cc1]

3-bromodibenzo[b,d]thiophene (42.8 g, 162.5 mmol), toluene(1550 mL), [1,1′-biphenyl]-4-amine (25 g, 147.7 mmol), Pd₂(dba)₃ (6.76 g, 162.5 mmol), P(t-Bu)₃ (3 g, 14.8 mmol), NaOt-Bu (42.6 g, 443.2 mmol) were reacted using the synthesis method of Sub 2-1 to give 37.9 g of the product (yield: 73%).

Examples of Sub 2 include, but are not limited to, the following.

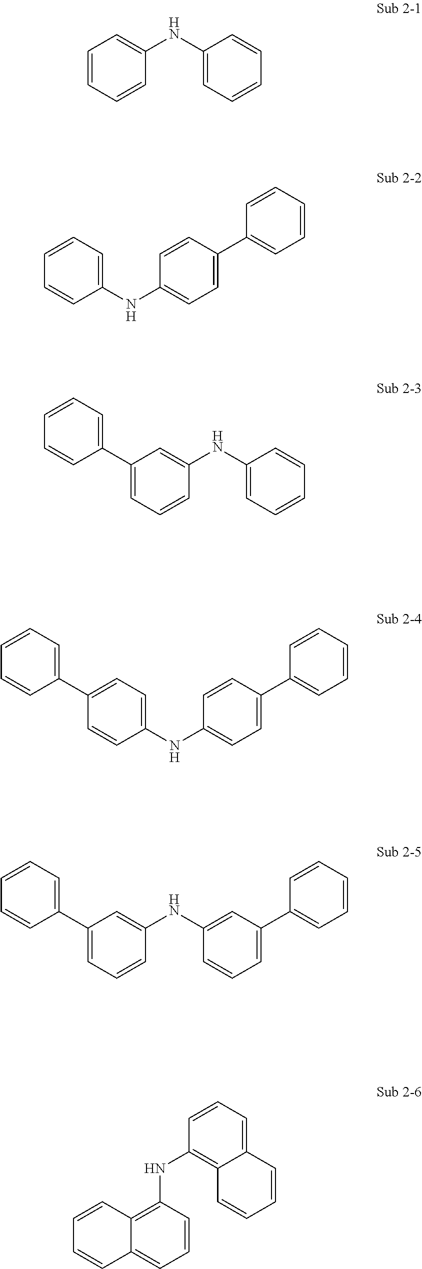 [SMILES: c1ccc(Nc2ccccc2)cc1 ; c1ccc(Nc2ccc(-c3ccccc3)cc2)cc1 ; c1ccc(Nc2cccc(-c3ccccc3)c2)cc1 ; c1ccc(-c2ccc(Nc3ccc(-c4ccccc4)cc3)cc2)cc1 ; c1ccc(-c2cccc(Nc3cccc(-c4ccccc4)c3)c2)cc1 ; c1ccc2c(Nc3cccc4ccccc34)cccc2c1]

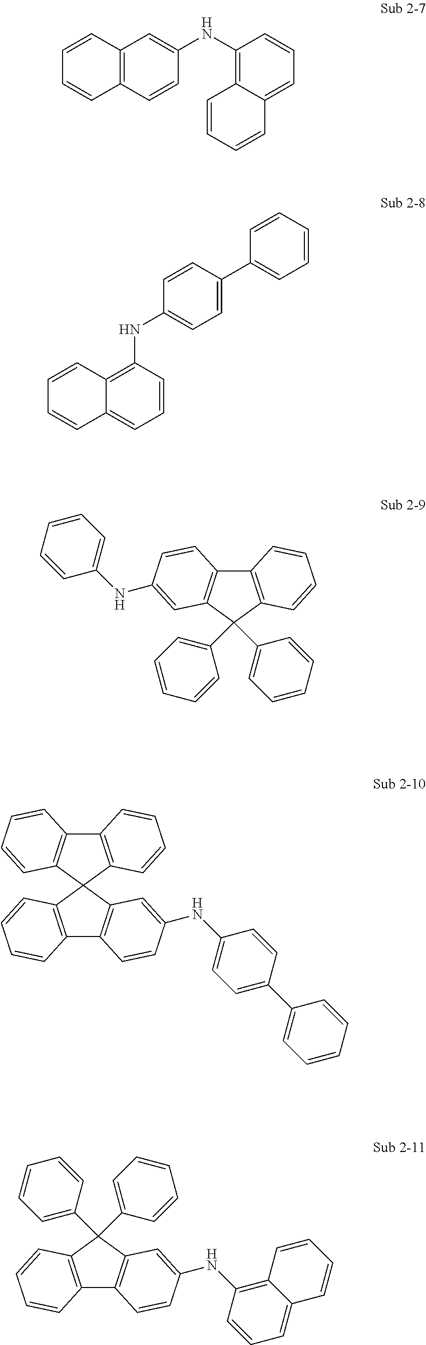 [SMILES: c1ccc2cc(Nc3cccc4ccccc34)ccc2c1 ; c1ccc(-c2ccc(Nc3cccc4ccccc34)cc2)cc1 ; c1ccc(Nc2ccc3c(c2)C(c2ccccc2)(c2ccccc2)c2ccccc2-3)cc1 ; c1ccc(-c2ccc(Nc3ccc4c(c3)C3(c5ccccc5-c5ccccc53)c3ccccc3-4)cc2)cc1 ; c1ccc(C2(c3ccccc3)c3ccccc3-c3ccc(Nc4cccc5ccccc45)cc32)cc1]

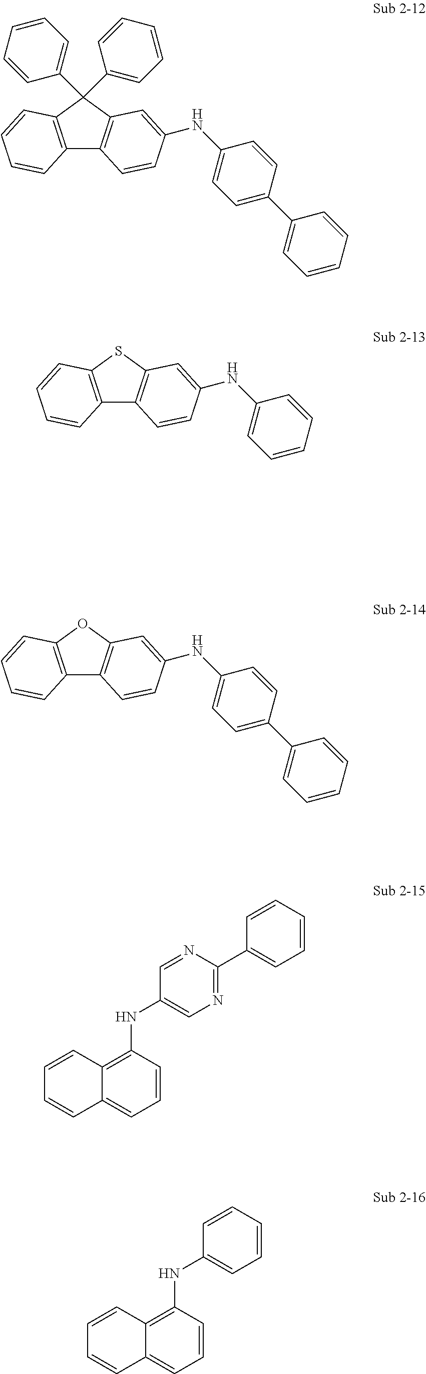 [SMILES: c1ccc(-c2ccc(Nc3ccc4c(c3)C(c3ccccc3)(c3ccccc3)c3ccccc3-4)cc2)cc1 ; c1ccc(Nc2ccc3c(c2)sc2ccccc23)cc1 ; c1ccc(-c2ccc(Nc3ccc4c(c3)oc3ccccc34)cc2)cc1 ; c1ccc(-c2ncc(Nc3cccc4ccccc34)cn2)cc1 ; c1ccc(Nc2cccc3ccccc23)cc1]

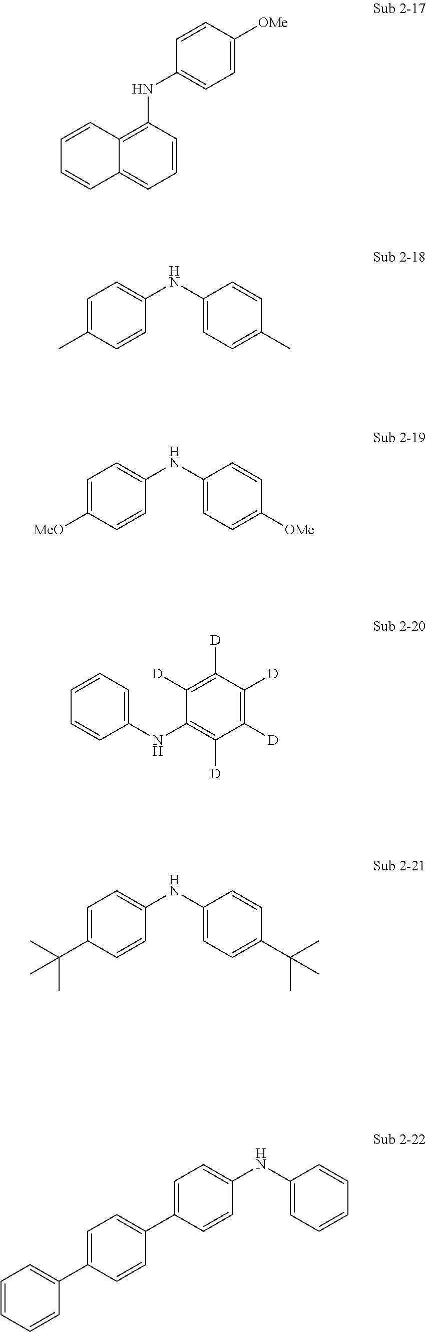

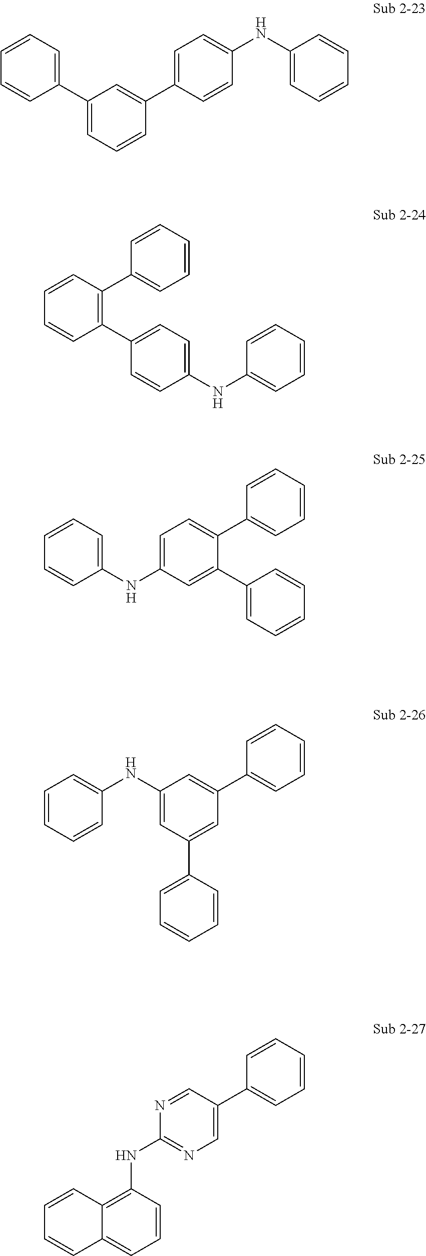 [SMILES: c1ccc(Nc2ccc(-c3cccc(-c4ccccc4)c3)cc2)cc1 ; c1ccc(Nc2ccc(-c3ccccc3-c3ccccc3)cc2)cc1 ; c1ccc(Nc2ccc(-c3ccccc3)c(-c3ccccc3)c2)cc1 ; c1ccc(Nc2cc(-c3ccccc3)cc(-c3ccccc3)c2)cc1 ; c1ccc(-c2cnc(Nc3cccc4ccccc34)nc2)cc1]

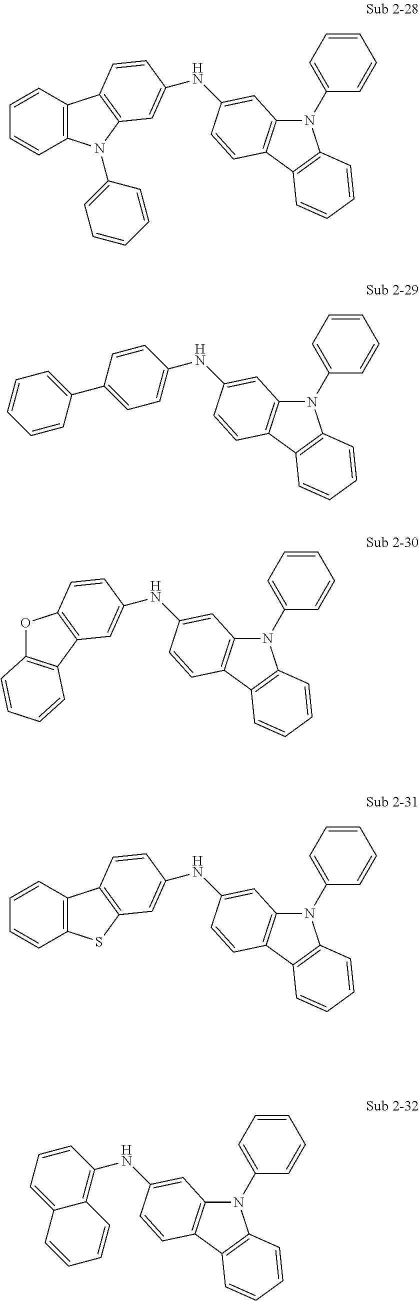 [SMILES: c1ccc(-n2c3ccccc3c3ccc(Nc4ccc5c6ccccc6n(-c6ccccc6)c5c4)cc32)cc1 ; c1ccc(-c2ccc(Nc3ccc4c5ccccc5n(-c5ccccc5)c4c3)cc2)cc1 ; c1ccc(-n2c3ccccc3c3ccc(Nc4ccc5oc6ccccc6c5c4)cc32)cc1 ; c1ccc(-n2c3ccccc3c3ccc(Nc4ccc5c(c4)sc4ccccc45)cc32)cc1 ; c1ccc(-n2c3ccccc3c3ccc(Nc4cccc5ccccc45)cc32)cc1]

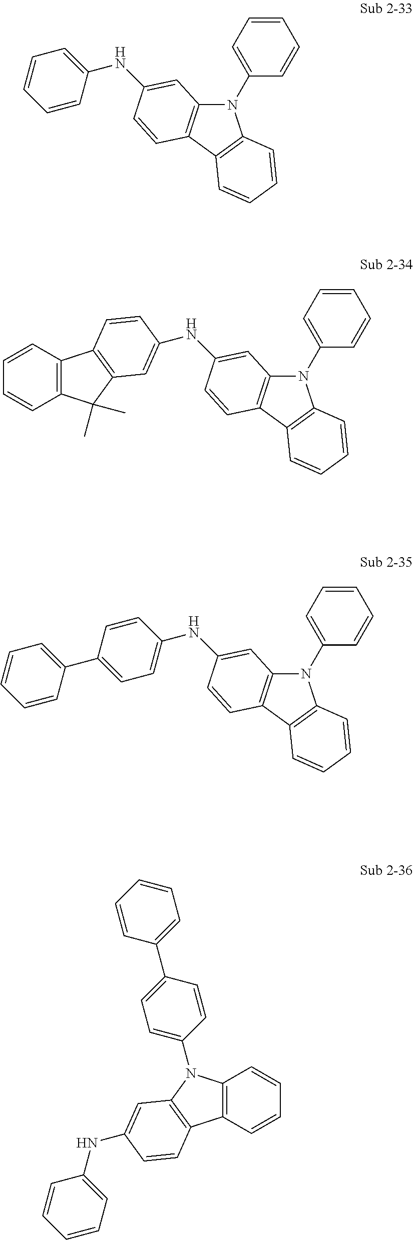 [SMILES: c1ccc(Nc2ccc3c4ccccc4n(-c4ccccc4)c3c2)cc1 ; CC1(C)c2ccccc2-c2ccc(Nc3ccc4c5ccccc5n(-c5ccccc5)c4c3)cc21 ; c1ccc(-c2ccc(Nc3ccc4c5ccccc5n(-c5ccccc5)c4c3)cc2)cc1 ; c1ccc(Nc2ccc3c4ccccc4n(-c4ccc(-c5ccccc5)cc4)c3c2)cc1]

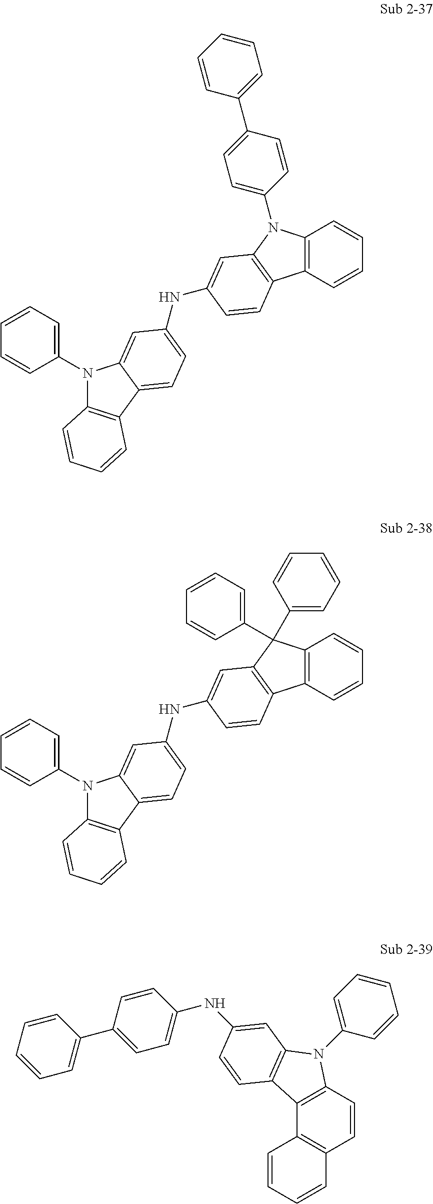 [SMILES: c1ccc(-c2ccc(-n3c4ccccc4c4ccc(Nc5ccc6c7ccccc7n(-c7ccccc7)c6c5)cc43)cc2)cc1 ; c1ccc(-n2c3ccccc3c3ccc(Nc4ccc5c(c4)C(c4ccccc4)(c4ccccc4)c4ccccc4-5)cc32)cc1 ; c1ccc(-c2ccc(Nc3ccc4c5c6ccccc6ccc5n(-c5ccccc5)c4c3)cc2)cc1]

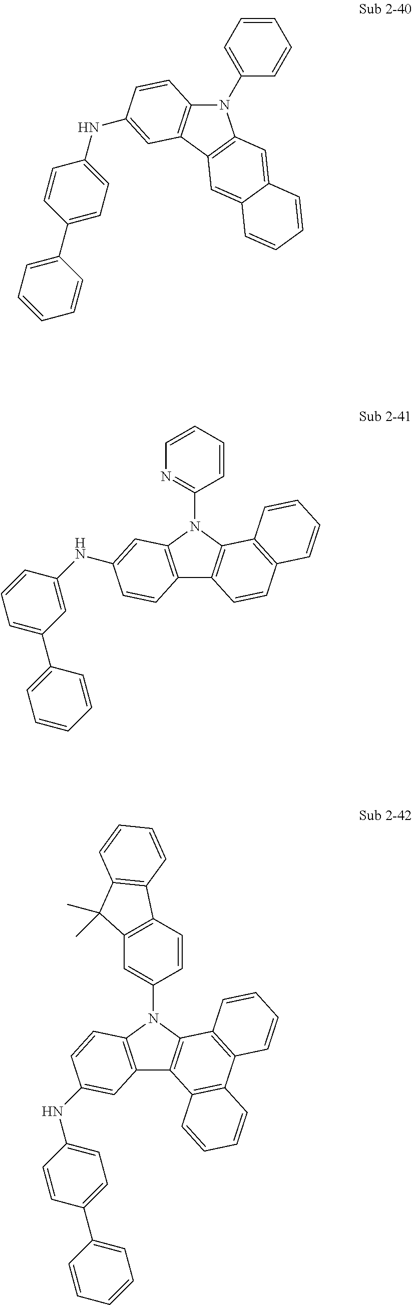 [SMILES: c1ccc(-c2ccc(Nc3ccc4c(c3)c3cc5ccccc5cc3n4-c3ccccc3)cc2)cc1 ; c1ccc(-c2cccc(Nc3ccc4c5ccc6ccccc6c5n(-c5ccccn5)c4c3)c2)cc1 ; CC1(C)c2ccccc2-c2ccc(-n3c4ccc(Nc5ccc(-c6ccccc6)cc5)cc4c4c5ccccc5c5ccccc5c43)cc21]

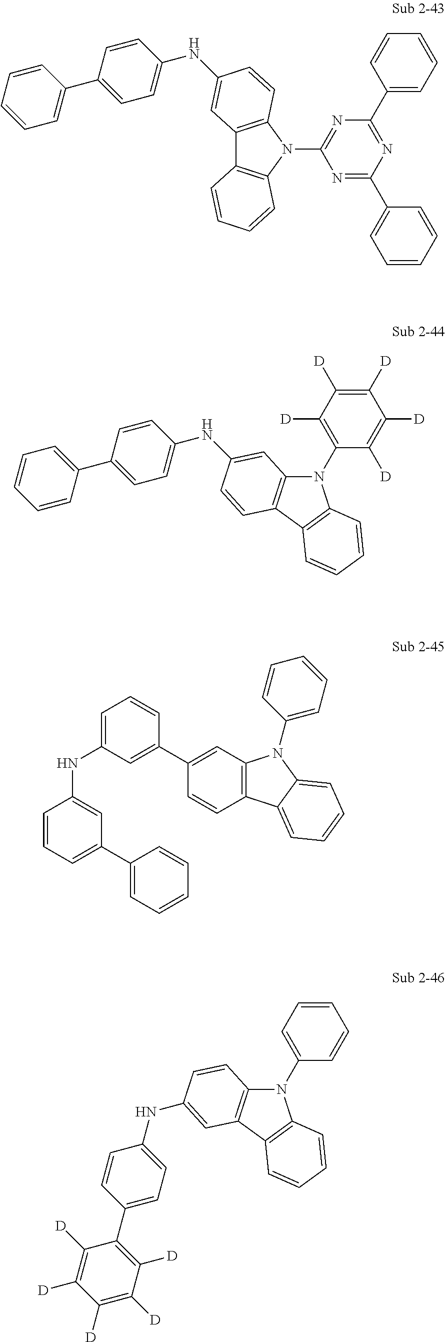 [SMILES: c1ccc(-c2ccc(Nc3ccc4c(c3)c3ccccc3n4-c3nc(-c4ccccc4)nc(-c4ccccc4)n3)cc2)cc1 ; [2H]c1c([2H])c([2H])c(-n2c3ccccc3c3ccc(Nc4ccc(-c5ccccc5)cc4)cc32)c([2H])c1[2H] ; c1ccc(-c2cccc(Nc3cccc(-c4ccc5c6ccccc6n(-c6ccccc6)c5c4)c3)c2)cc1 ; [2H]c1c([2H])c([2H])c(-c2ccc(Nc3ccc4c(c3)c3ccccc3n4-c3ccccc3)cc2)c([2H])c1[2H]]

TABLE 2

| compound | FD-MS | compound | FD-MS |
|---|---|---|---|
| Sub 2-1 | m/z = 169.09(C₁₂H₁₁N = 169.22) | Sub 2-2 | m/z = 245.12(C₁₈H₁₅N = 245.32) |
| Sub 2-3 | m/z = 245.12(C₁₈H₁₅N = 245.32) | Sub 2-4 | m/z = 321.15(C₂₄H₁₉N = 321.41) |
| Sub 2-5 | m/z = 321.15 (C₂₄H₁₉N = 321.41) | Sub 2-6 | m/z = 269.12(C₂₀H₁₅N = 269.34) |
| Sub 2-7 | m/z = 269.12(C₂₀H₁₅N = 269.34) | Sub 2-8 | m/z = 295.14(C₂₂H₁₇N = 295.38) |
| Sub 2-9 | m/z = 409.18(C₃₁H₂₃N = 409.52) | Sub 2-10 | m/z = 483.20(C₃₇H₂₅N = 483.60) |
| Sub 2-11 | m/z = 459.20(C₃₅H₂₅N = 459.58) | Sub 2-12 | m/z = 485.21(C₃₇H₂₇N = 485.62) |
| Sub 2-13 | m/z = 275.08(C₁₈H₁₃NS = 275.37) | Sub 2-14 | m/z = 335.13(C₂₄H₁₇NO = 335.40) |
| Sub 2-15 | m/z = 297.13(C₂₀H₁₅N₃ = 297.35) | Sub 2-16 | m/z = 219.10(C₁₆H₁₃N = 219.28) |
| Sub 2-17 | m/z = 249.12(C₁₇H₁₅NO = 249.31) | Sub 2-18 | m/z = 197.12(C₁₄H₁₅N = 197.28) |
| Sub 2-19 | m/z = 229.11(C₁₄H₁₅NO₂ = 229.27) | Sub 2-20 | m/z = 174.12(C₁₂H₆D₅N = 174.25) |
| Sub 2-21 | m/z = 281.21(C₂₀H₂₇N = 281.44) | Sub 2-22 | m/z = 321.15(C₂₄H₁₉N = 321.41) |
| Sub 2-23 | m/z = 321.15(C₂₄H₁₉N = 321.41) | Sub 2-24 | m/z = 321.15(C₂₄H₁₉N = 321.41) |
| Sub 2-25 | m/z = 321.15(C₂₄H₁₉N = 321.41) | Sub 2-26 | m/z = 321.15(C₂₄H₁₉N = 321.41) |
| Sub 2-27 | m/z = 297.13(C₂₀H₁₅N₃ = 297.35) | Sub 2-28 | m/z = 499.20(C₃₆H₂₅N₃ = 499.60) |
| Sub 2-29 | m/z = 499.20(C₃₆H₂₂N₂ = 410.51) | Sub 2-30 | m/z = 424.16(C₃₀H₂₀N₂O = 424.49) |
| Sub 2-31 | m/z = 440.13(C₃₀H₂₀N₂S = 440.56) | Sub 2-32 | m/z = 384.16(C₂₈H₂₀N₂ = 384.47) |
| Sub 2-33 | m/z = 334.15(C₂₄H₁₈N₂ = 334.41) | Sub 2-34 | m/z = 450.21(C₃₃H₂₆N₂ = 450.57) |
| Sub 2-35 | m/z = 410.18(C₃₀H₂₂N₂ = 410.51) | Sub 2-36 | m/z = 410.18(C₃₀H₂₂N₂ = 410.51) |
| Sub 2-37 | m/z = 575.24(C₄₂H₂₉N₃ = 575.70) | Sub 2-38 | m/z = 574.24(C₄₃H₃₀N₂ = 574.71) |
| Sub 2-39 | m/z = 460.19(C₃₄H₂₄N₂ = 460.57) | Sub 2-40 | m/z = 460.19(C₃₄H₂₄N₂ = 460.57) |
| Sub 2-41 | m/z = 461.19(C₃₃H₂₃N₃ = 461.56) | Sub 2-42 | m/z = 626.27(C₄₇H₃₄N₂ = 626.79) |
| Sub 2-43 | m/z = 565.23(C₃₉H₂₇N₅ = 565.67) | Sub 2-44 | m/z = 415.21(C₃₀H₁₇D₅N₂ = 415.54) |
| Sub 2-45 | m/z = 486.21(C₃₆H₂₆N₂ = 486.61) | Sub 2-46 | m/z = 415.21(C₃₀H₁₇D₅N₂ = 415.54) |

Synthesis Example of Final Products 1

Synthesis Example of 1-1

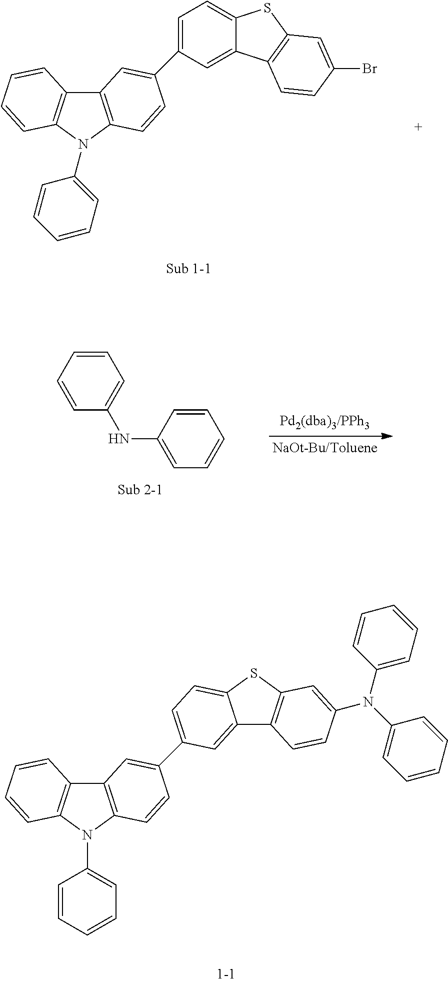 [SMILES: Brc1ccc2c(c1)sc1ccc(-c3ccc4c(c3)c3ccccc3n4-c3ccccc3)cc12 ; c1ccc(Nc2ccccc2)cc1 ; c1ccc(N(c2ccccc2)c2ccc3c(c2)sc2ccc(-c4ccc5c(c4)c4ccccc4n5-c4ccccc4)cc23)cc1]

Sub 2-1 (8.0 g, 47.3 mmol) was added to a round bottom flask and dissolved in toluene (500 mL), and Sub 1-1 (26.2 g, 52.0 mmol), Pd₂(dba)₃ (2.2 g, 2.4 mmol), P(t-Bu)₃ (1 g, 4.73 mmol), NaOt-Bu (13.6 g, 141.8 mmol) were added and stirred at 100° C. After the reaction was completed, the reaction mixture was extracted with CH₂Cl₂ and water. The organic layer was dried over MgSO₄ and concentrated. The resulting compound was separated by silicagel column chromatography and recrystallized to obtain 25.0 g of the product. (yield: 81%)

Synthesis Example of 1-12

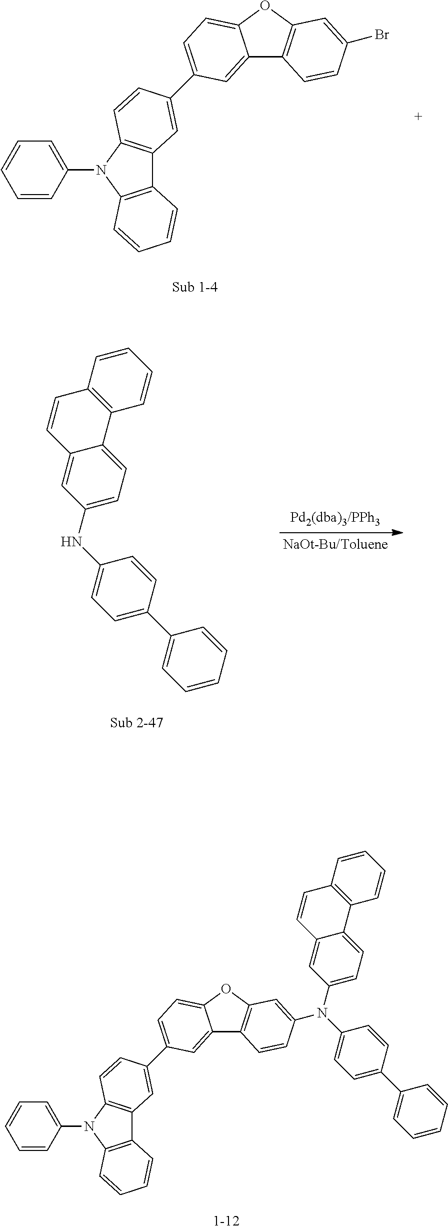 [SMILES: Brc1ccc2c(c1)oc1ccc(-c3ccc4c(c3)c3ccccc3n4-c3ccccc3)cc12 ; c1ccc(-c2ccc(Nc3ccc4c(ccc5ccccc54)c3)cc2)cc1 ; c1ccc(-c2ccc(N(c3ccc4c(ccc5ccccc54)c3)c3ccc4c(c3)oc3ccc(-c5ccc6c(c5)c5ccccc5n6-c5ccccc5)cc34)cc2)cc1]

Sub 2-47 (16.3 g, 47.3 mmol) and Sub 1-4 (25.4 g, 52.0 mmol) were reacted using the synthesis method of 1-1 to give 30.5 g of the product (yield: 78%).

Synthesis Example of 1-20

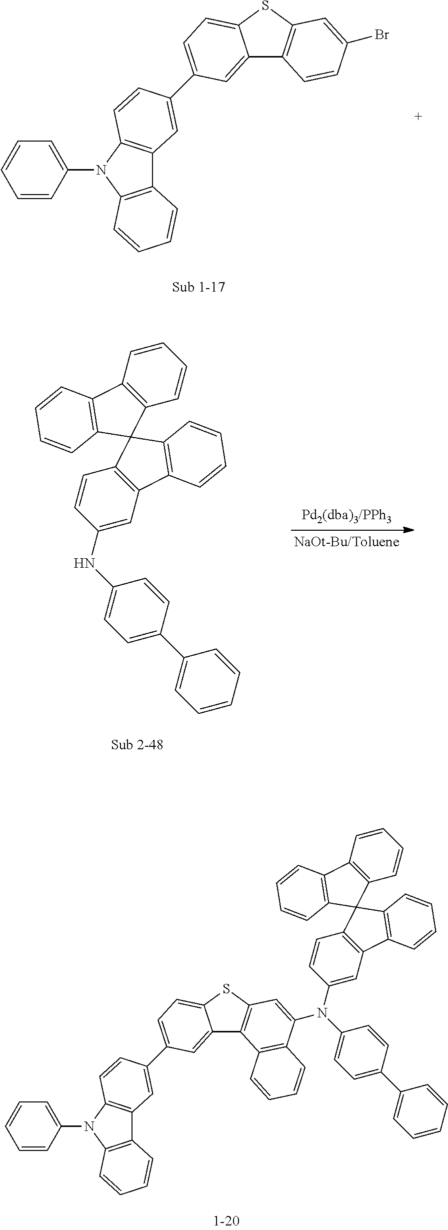 [SMILES: Brc1ccc2c(c1)sc1ccc(-c3ccc4c(c3)c3ccccc3n4-c3ccccc3)cc12 ; c1ccc(-c2ccc(Nc3ccc4c(c3)-c3ccccc3C43c4ccccc4-c4ccccc43)cc2)cc1 ; c1ccc(-c2ccc(N(c3ccc4c(c3)-c3ccccc3C43c4ccccc4-c4ccccc43)c3cc4sc5ccc(-c6ccc7c(c6)c6ccccc6n7-c6ccccc6)cc5c4c4ccccc34)cc2)cc1]

Sub 2-48 (22.9 g, 47.3 mmol), Sub 1-17 (28.8 g, 52.0 mmol) were reacted using the synthesis method of 1-1 to give 35.8 g of the product (yield: 72%).

Synthesis Example of 2-5

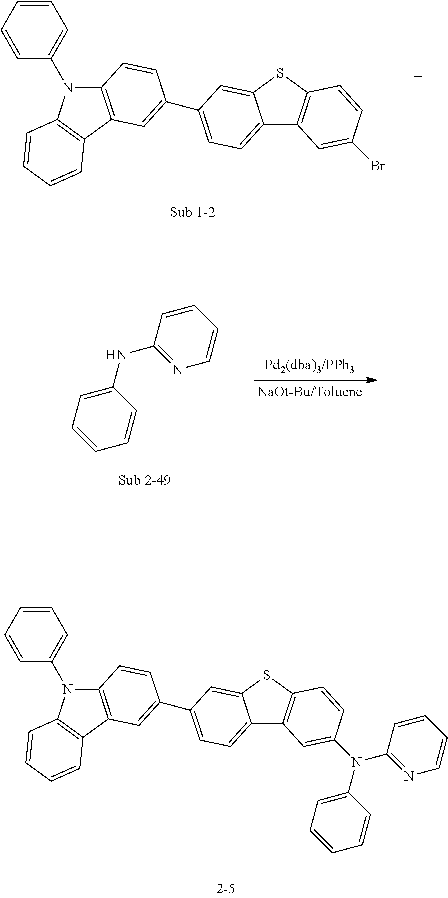 [SMILES: Brc1ccc2sc3cc(-c4ccc5c(c4)c4ccccc4n5-c4ccccc4)ccc3c2c1 ; c1ccc(Nc2ccccn2)cc1 ; c1ccc(N(c2ccc3sc4cc(-c5ccc6c(c5)c5ccccc5n6-c5ccccc5)ccc4c3c2)c2ccccn2)cc1]

Sub 2-49 (8.1 g, 47.3 mmol), Sub 1-2 (26.2 g, 52.0 mmol) were reacted using the synthesis method of 1-1 to give 23.5 g of the product (yield: 76%).

Synthesis Example of 2-16

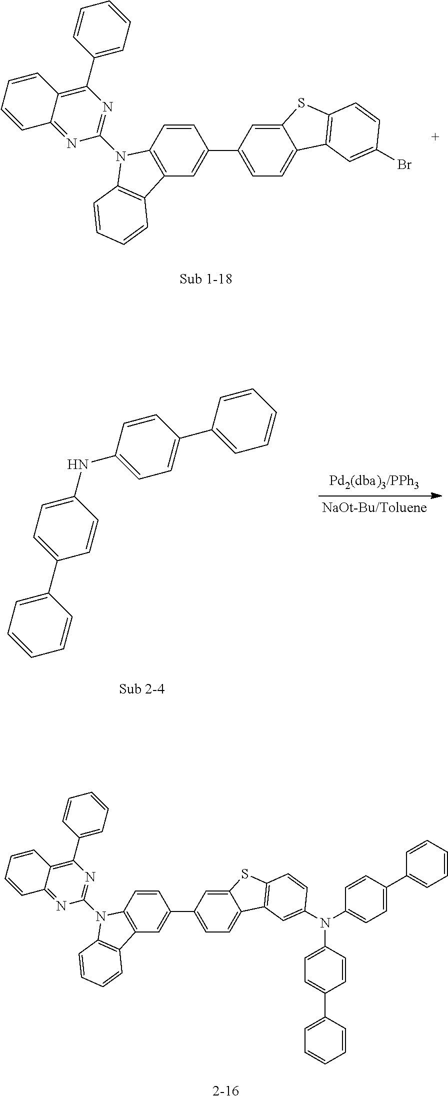 [SMILES: Brc1ccc2sc3cc(-c4ccc5c(c4)c4ccccc4n5-c4nc(-c5ccccc5)c5ccccc5n4)ccc3c2c1 ; c1ccc(-c2ccc(Nc3ccc(-c4ccccc4)cc3)cc2)cc1 ; c1ccc(-c2ccc(N(c3ccc(-c4ccccc4)cc3)c3ccc4sc5cc(-c6ccc7c(c6)c6ccccc6n7-c6nc(-c7ccccc7)c7ccccc7n6)ccc5c4c3)cc2)cc1]

Sub 2-4 (15.2 g, 47.3 mmol), Sub 1-18 (32.9 g, 52.0 mmol) were reacted using the synthesis method of 1-1 to give 35.9 g of the product (yield: 79%).

Synthesis Example of 2-21

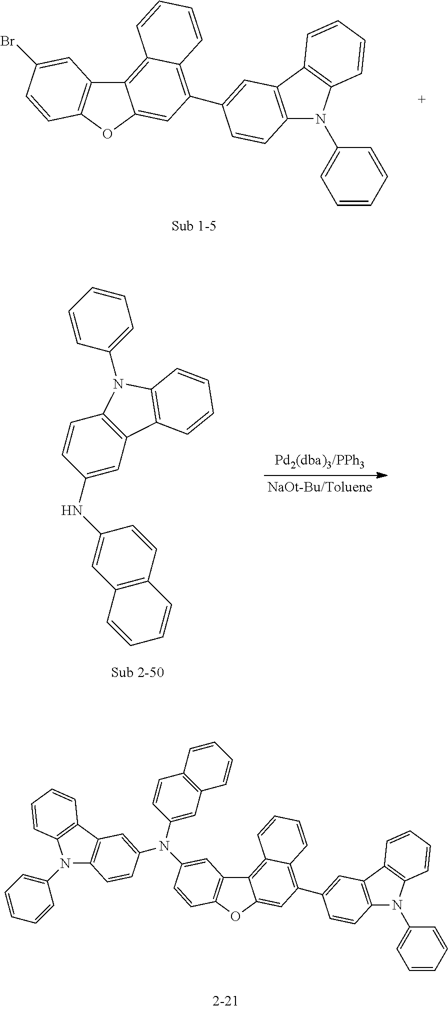 [SMILES: Brc1ccc2oc3cc(-c4ccc5c(c4)c4ccccc4n5-c4ccccc4)c4ccccc4c3c2c1 ; c1ccc(-n2c3ccccc3c3cc(Nc4ccc5ccccc5c4)ccc32)cc1 ; c1ccc(-n2c3ccccc3c3cc(-c4cc5oc6ccc(N(c7ccc8ccccc8c7)c7ccc8c(c7)c7ccccc7n8-c7ccccc7)cc6c5c5ccccc45)ccc32)cc1]

Sub 2-50 (18.2 g, 47.3 mmol), Sub 1-5 (28.0 g, 52.0 mmol) were reacted using the synthesis method of 1-1 to give 35.5 g of the product (yield: 81%).

TABLE 3

| compound | FD-MS | compound | FD-MS |
|---|---|---|---|
| 1-1 | m/z = 592.20(C₄₂H₂₈N₂S = 592.76) | 1-2 | m/z = 626.24(C₄₆H₃₀N₂O = 626.76) |
| 1-3 | m/z = 626.24(C₄₆H₃₀N₂O = 626.76) | 1-4 | m/z = 744.26(C₅₄H₃₆N₂S = 744.96) |
| 1-5 | m/z = 593.19(C₄₁H₂₇N₃S = 593.75) | 1-6 | m/z = 708.26(C₅₁H₃₆N₂S = 708.92) |
| 1-7 | m/z = 816.31(C₆₁H₄₀N₂O = 817.00) | 1-8 | m/z = 890.33(C₆₇H₄₂N₂O = 891.09) |
| 1-9 | m/z = 807.27(C₅₈H₃₇N₃S = 808.02) | 1-10 | m/z = 774.22(C₅₄H₃₄N₂S₂ = 775.00) |
| 1-11 | m/z = 682.21(C₄₈H₃₀N₂OS = 682.84) | 1-12 | m/z = 752.28(C₅₆H₃₆N₂O = 752.92) |
| 1-13 | m/z = 626.24(C₄₆H₃₀N₂O = 626.76) | 1-14 | m/z = 692.23(C₅₀H₃₂N₂S = 692.88) |
| 1-15 | m/z = 718.24(C₅₂H₃₄N₂S = 718.92) | 1-16 | m/z = 872.30(C₆₂H₄₀N₄S = 873.09) |
| 1-17 | m/z = 677.25(C₄₉H₃₁N₃O = 677.81) | 1-18 | m/z = 808.29(C₅₉H₄₀N₂S = 809.04) |
| 1-19 | m/z = 882.31(C₆₅H₄₂N₂S = 883.13) | 1-20 | m/z = 956.32(C₇₁H₄₄N₂S = 957.21) |
| 1-21 | m/z = 841.31(C₆₂H₃₉N₃O = 842.01) | 1-22 | m/z = 824.23(C₅₈H₃₆N₂S₂ = 825.06) |
| 1-23 | m/z = 716.25(C₅₂H₃₂N₂O₂ = 716.84) | 1-24 | m/z = 868.29(C₆₄H₄₀N₂S = 869.10) |
| 2-1 | m/z = 592.20(C₄₂H₂₈N₂S = 592.76) | 2-2 | m/z = 626.24(C₄₆H₃₀N₂O = 626.76) |
| 2-3 | m/z = 626.24(C₄₆H₃₀N₂O = 626.76) | 2-4 | m/z = 744.26(C₅₄H₃₆N₂S = 744.96) |
| 2-5 | m/z = 593.19(C₄₁H₂₇N₃S = 593.75) | 2-6 | m/z = 708.26(C₅₁H₃₆N₂S = 708.92) |
| 2-7 | m/z = 816.31(C₆₁H₄₀N₂O = 817.00) | 2-8 | m/z = 890.33(C₆₇H₄₂N₂O = 891.09) |
| 2-9 | m/z = 807.27(C₅₈H₃₇N₃S = 808.02) | 2-10 | m/z = 774.22(C₅₄H₃₄N₂S₂ = 775.00) |
| 2-11 | m/z = 682.21(C₄₈H₃₀N₂OS = 682.84) | 2-12 | m/z = 752.28(C₅₆H₃₆N₂O = 752.92) |
| 2-13 | m/z = 626.24(C₄₆H₃₀N₂O = 626.76) | 2-14 | m/z = 692.23(C₅₀H₃₂N₂S = 692.88) |
| 2-15 | m/z = 718.24(C₅₂H₃₄N₂S = 718.92) | 2-16 | m/z = 872.30(C₆₂H₄₀N₄S = 873.09) |
| 2-17 | m/z = 677.25(C₄₉H₃₁N₃O = 677.81) | 2-18 | m/z = 808.29(C₅₉H₄₀N₂S = 809.04) |
| 2-19 | m/z = 882.31(C₆₅H₄₂N₂S = 883.13) | 2-20 | m/z = 956.32(C₇₁H₄₄N₂S = 957.21) |
| 2-21 | m/z = 841.31(C₆₂H₃₉N₃O = 842.01) | 2-22 | m/z = 824.23(C₅₈H₃₆N₂S₂ = 825.06) |
| 2-23 | m/z = 716.25(C₅₂H₃₂N₂O₂ = 716.84) | 2-24 | m/z = 868.29(C₆₄H₄₀N₂S = 869.10) |

### SYNTHESIS EXAMPLE 2

The final product 2 represented by Formula (13) of the present invention can be synthesized by reacting Sub 3 or Sub 4 with Sub 5 as illustrated in the following Reaction Scheme 4.

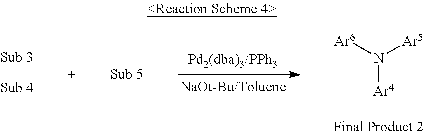

1.Synthesis Example of Sub 3

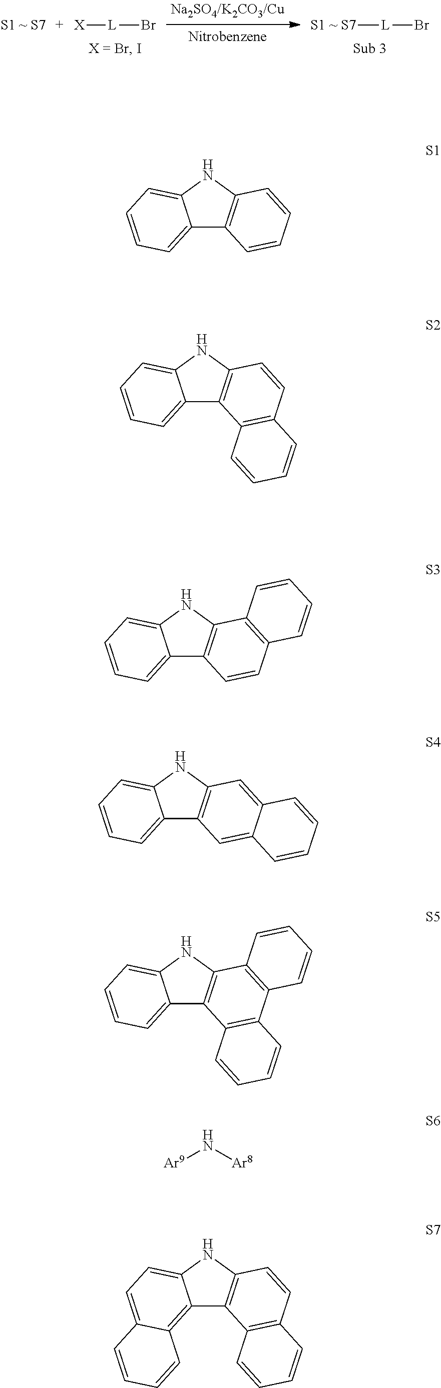

* L is L³ or L⁵ defined in Formulas (2-a), (2-b) and (2-c).

1) Synthesis Example of Sub 3-1-1(L=biphenyl)

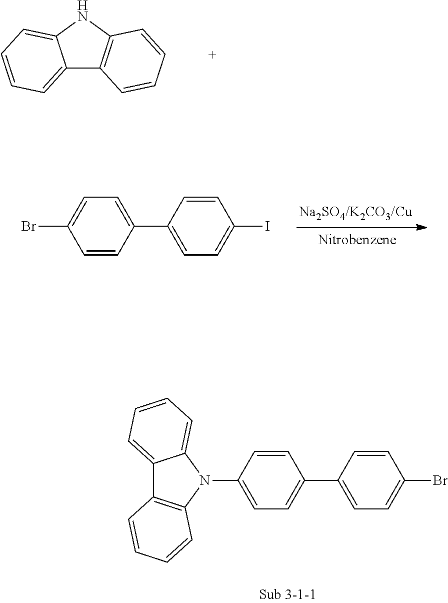 [SMILES: c1ccc2c(c1)[nH]c1ccccc12 ; Brc1ccc(-c2ccc(I)cc2)cc1 ; Brc1ccc(-c2ccc(-n3c4ccccc4c4ccccc43)cc2)cc1]

Starting material 9H-carbazole (50.16 g, 300 mmol) and 4-bromo-4′-iodo-1,1′-biphenyl (129.2 g, 360 mmol), Na₂SO₄ (42.6 g, 300 mmol), K₂CO₃ (41.4 g, 300 mmol), Cu (5.72 g, 90 mmol), nitrobenzene were reacted using the above synthesis method to give 80.05 g of the product. (yield: 67%).

2) Synthesis Example of Sub 3-1-2 (L=9,9-dimethyl-9H-fluorene)

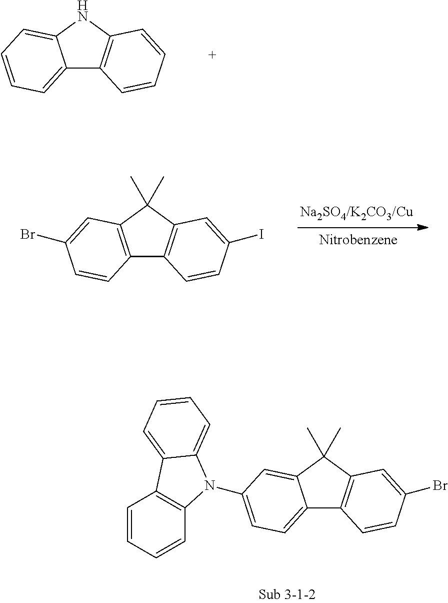 [SMILES: c1ccc2c(c1)[nH]c1ccccc12 ; CC1(C)c2cc(Br)ccc2-c2ccc(I)cc21 ; CC1(C)c2cc(Br)ccc2-c2ccc(-n3c4ccccc4c4ccccc43)cc21]

Starting material 9H-carbazole (50.16 g, 300 mmol) and 2-bromo-7-iodo-9,9-dimethyl-9H-fluorene (143.7 g, 360 mmol), Na₂SO₄ (42.6 g, 300 mmol), K₂CO₃ (41.4 g, 300 mmol), Cu (5.72 g, 90 mmol), nitrobenzene were reacted using the above synthesis method to give 88.11 g of the product. (yield: 67%).

3) Synthesis Example of Sub 3-1-3 (L=9,9-dimethyl-9H-fluorene)

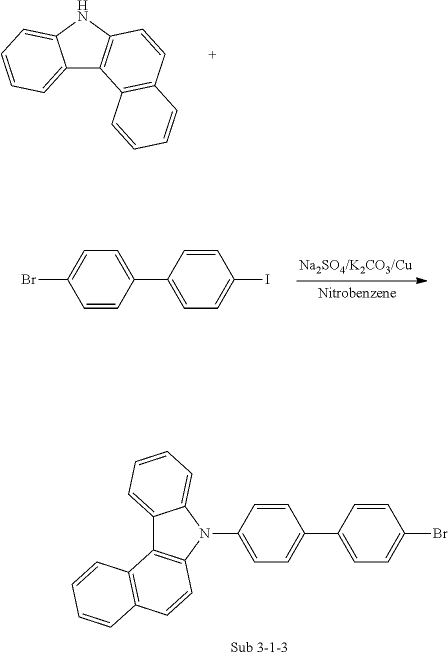 [SMILES: c1ccc2c(c1)ccc1[nH]c3ccccc3c12 ; Brc1ccc(-c2ccc(I)cc2)cc1 ; Brc1ccc(-c2ccc(-n3c4ccccc4c4c5ccccc5ccc43)cc2)cc1]

Starting material 7H-benzo[c]carbazole (65.18 g, 300 mmol) and 4-bromo-4′-iodo-1,1′-biphenyl (129.2 g, 360 mmol), Na₂SO₄ (42.6 g, 300 mmol), K₂CO₃ (41.4 g, 300 mmol), Cu (5.72 g, 90 mmol), nitrobenzene were reacted using the above synthesis method to give 92.8 g of the product. (yield: 69%).

4) Synthesis Example of Sub 3-1-4 (L=9,9-dimethyl-9H-fluorene)

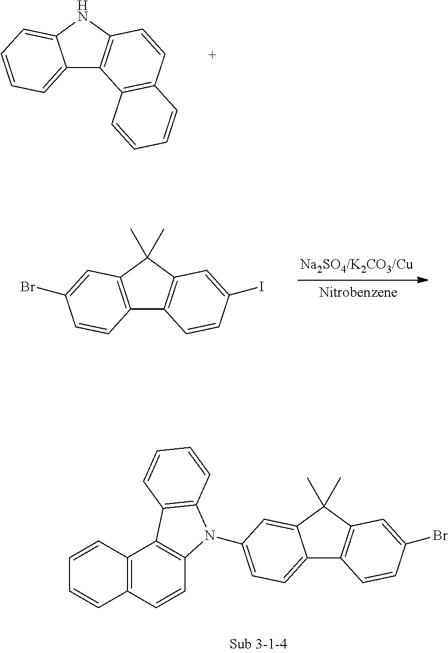 [SMILES: c1ccc2c(c1)ccc1[nH]c3ccccc3c12 ; CC1(C)c2cc(Br)ccc2-c2ccc(I)cc21 ; CC1(C)c2cc(Br)ccc2-c2ccc(-n3c4ccccc4c4c5ccccc5ccc43)cc21]

Starting material 7H-benzo[c]carbazole (65.18 g, 300 mmol) and 2-bromo-7-iodo-9,9-dimethyl-9H-fluorene (143.7 g, 360 mmol), Na₂SO₄ (42.6 g, 300 mmol), K₂CO₃ (41.4 g, 300 mmol), Cu (5.72 g, 90 mmol), nitrobenzene were reacted using the above synthesis method to give 95.24 g of the product. (yield: 65%).

5) Synthesis Example of Sub 3-1-5 (L=biphenyl)

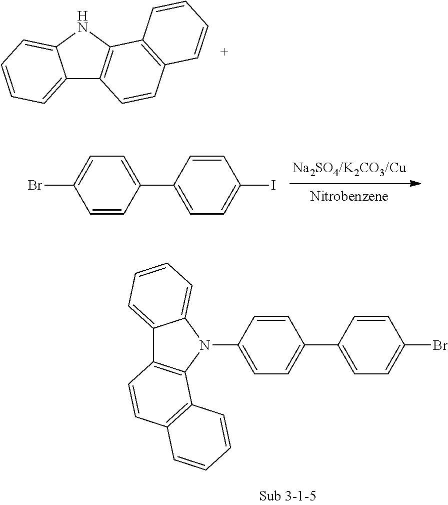 [SMILES: c1ccc2c(c1)ccc1c3ccccc3[nH]c21 ; Brc1ccc(-c2ccc(I)cc2)cc1 ; Brc1ccc(-c2ccc(-n3c4ccccc4c4ccc5ccccc5c43)cc2)cc1]

Starting material 11H-benzo[a]carbazole (65.18 g, 300 mmol) and 4-bromo-4′-iodo-1,1′-biphenyl (129.2 g, 360 mmol), Na₂SO₄ (42.6 g, 300 mmol), K₂CO₃ (41.4 g, 300 mmol), Cu (5.72 g, 90 mmol), nitrobenzene were reacted using the above synthesis method to give 80.05 g of the product. (yield: 62%).

6) Synthesis Example of Sub 3-1-6 (L=9,9-dimethyl-9H-fluorene)

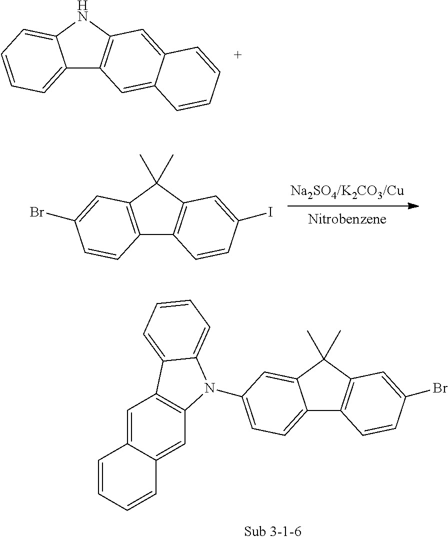

Starting material 5H-benzo[b]carbazole (65.18 g, 300 mmol) and 2-bromo-7-iodo-9,9-dimethyl-9H-fluorene (143.7 g, 360 mmol), Na₂SO₄ (42.6 g, 300 mmol), K₂CO₃ (41.4 g, 300 mmol), Cu (5.72 g, 90 mmol), nitrobenzene were reacted using the above synthesis method to give 93.78 g of the product. (yield: 64%).

7) Synthesis Example of Sub 3-1-7 (L=biphenyl)

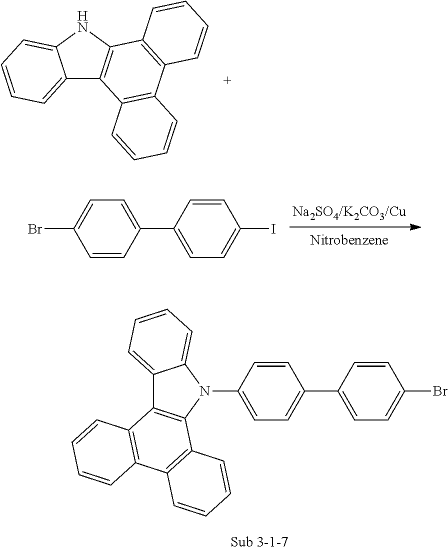

Starting material 9H-dibenzo[a,c]carbazole (80.2 g, 300 mmol) and 4-bromo-4′-iodo-1,1′-biphenyl (129.2 g, 360 mmol), Na₂SO₄ (42.6 g, 300 mmol), K₂CO₃ (41.4 g, 300 mmol), Cu (5.72 g, 90 mmol), nitrobenzene were reacted using the above synthesis method to give 98.7 g of the product. (yield: 66%).

8) Synthesis Example of Sub 3-1-8 (L=biphenyl)

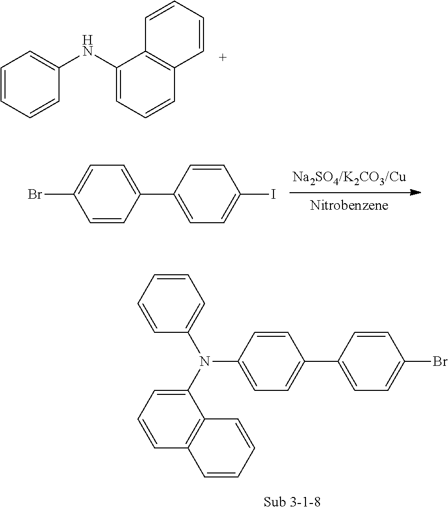 [SMILES: c1ccc(Nc2cccc3ccccc23)cc1 ; Brc1ccc(-c2ccc(I)cc2)cc1 ; Brc1ccc(-c2ccc(N(c3ccccc3)c3cccc4ccccc34)cc2)cc1]

Starting material N-phenylnaphthalen-1-amine (65.8 g, 300 mmol) and 4-bromo-4′-iodo-1,1′-biphenyl (129.2 g, 360 mmol), Na₂SO₄ (42.6 g, 300 mmol), K₂CO₃ (41.4 g, 300 mmol), Cu (5.72 g, 90 mmol), nitrobenzene were reacted using the above synthesis method to give 89.2 g of the product. (yield: 66%).

9) Synthesis Example of Sub 3-1-9 (L=9,9-dimethyl-9H-fluorene)

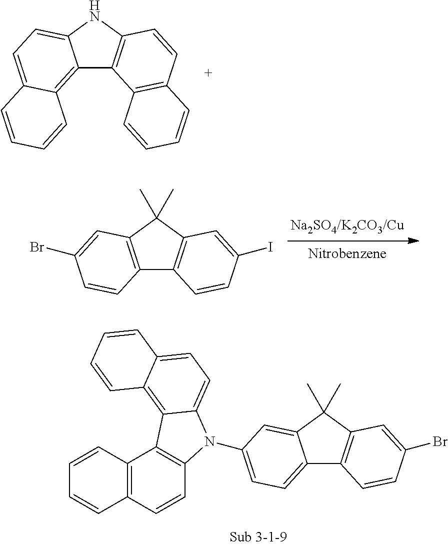

Starting material 7H-dibenzo[c,g]carbazole (80.2 g, 300 mmol) and 2-bromo-7-iodo-9,9-dimethyl-9H-fluorene (143.7 g, 360 mmol), Na₂SO₄ (42.6 g, 300 mmol), K₂CO₃ (41.4 g, 300 mmol), Cu (5.72 g, 90 mmol), nitrobenzene were reacted using the above synthesis method to give 98.5 g of the product. (yield: 61%).

2. Synthesis Example of Sub 4

Sub 4 of Reaction Scheme 4 can be synthesized by the reaction path of Reaction Scheme 5 below.

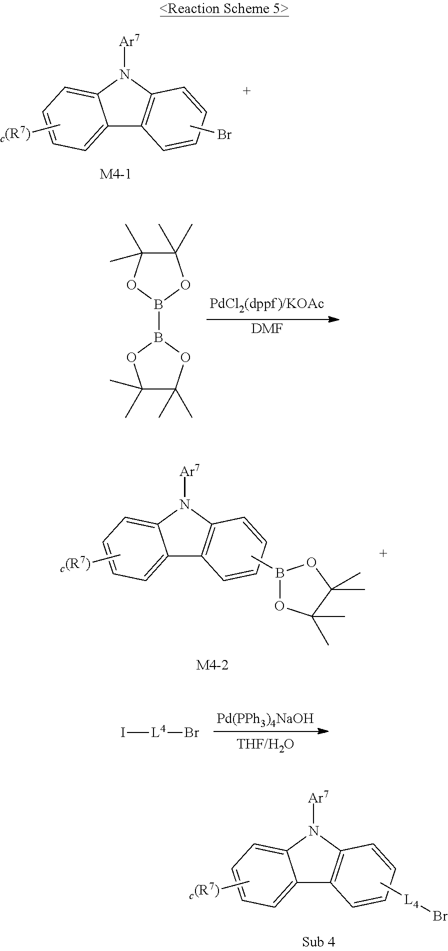

1) Synthesis Example of M4-2-1

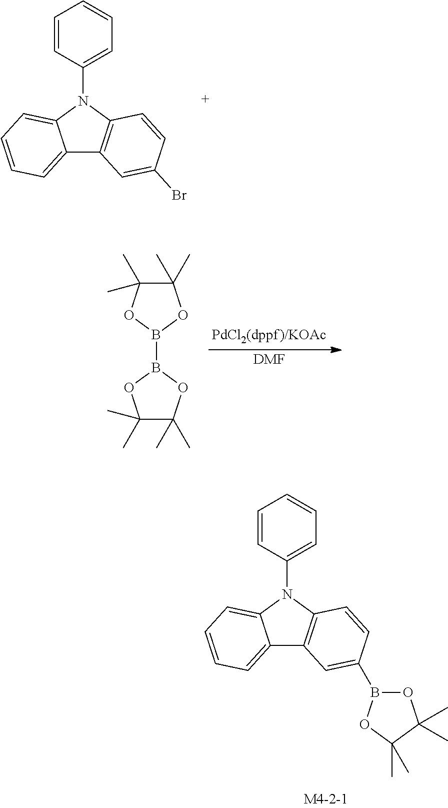

3-bromo-9-phenyl-9H-carbazole (45.1 g, 140 mmol) was dissolved in DMF 980 ml and Bispinacolborate (39.1 g, 154 mmol), PdCl₂(dppf) catalyst (3.43 g, 4.2 mmol), KOAc (41.3 g, 420 mmol) were added in order and stirred for 24 hours and then after synthesizing the borate compound, the obtained compound was separated over a silicagel column and recrystallization to give 35.2g of the borate compound (yield: 68%).

2) Synthesis Examnle of M4-2-2

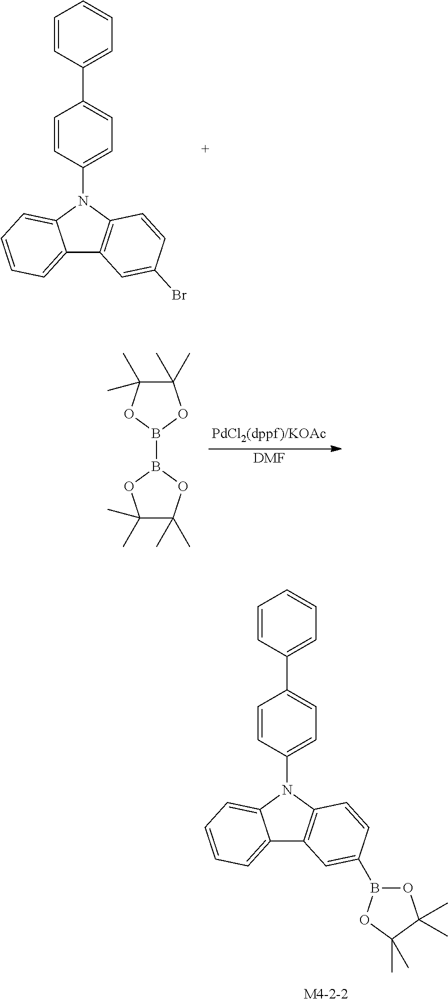

40 g (64%) was obtained through the same experimental procedure as M4-2-1.

3) Synthesis Example of Sub 4-1-1

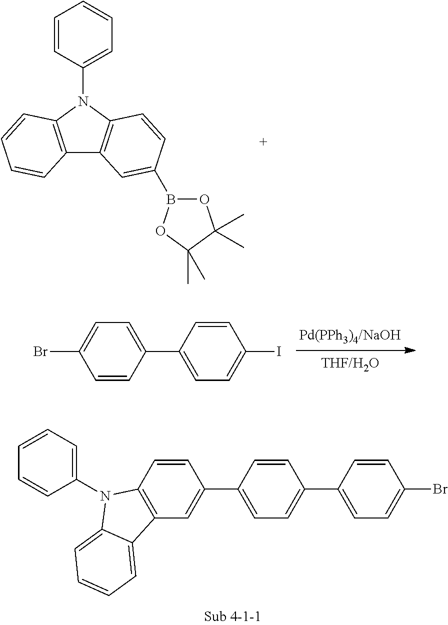

M4-2-1 (29.5 g, 80 mmol) was dissolved in THF 360 mL and 4-bromo-4′-iodo-1,1′-biphenyl (30.16 g, 84 mmol), Pd(PPh₃)₄ (2.8 g, 2.4 mmol), NaOH (9.6 g, 240 mmol), and water (180 mL) were added and refluxed with stirring. When the reaction was completed, the reaction mixture was extracted with ether and water. The organic layer was dried over MgSO₄ and concentrated. The resulting compound was separated by silicagel column chromatography and recrystallized to obtain 26.56 g (yield: 70%) of the product.

4) Synthesis Example of Sub 4-1-2

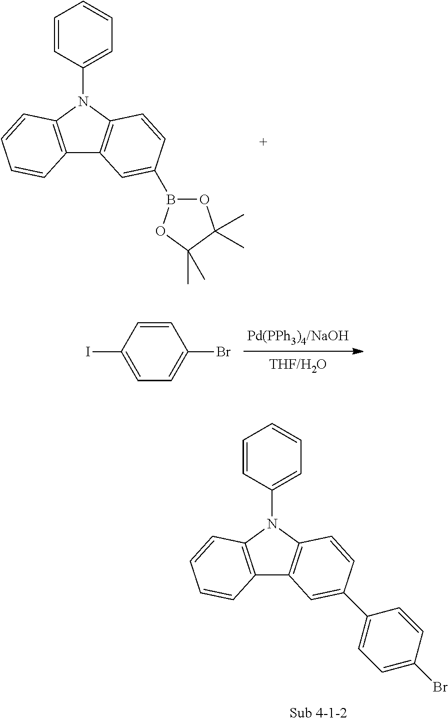

M4-2-1 (29.5 g, 80 mmol) was dissolved in THF 360 mL, and 1-bromo-4-iodobenzene (23.8 g, 84 mmol), Pd(PPh₃)₄ (2.8 g, 2.4 mmol), NaOH (9.6 g, 240 mmol), and water (180 mL) were added and refluxed with stirring. When the reaction was completed, the reaction mixture was extracted with ether and water. The organic layer was dried over MgSO₄ and concentrated. The resulting compound was separated by silicagel column chromatography and recrystallized to obtain 22.9 g (yield: 72%) of the product.

5) Synthesis Example of Sub 4-1-3

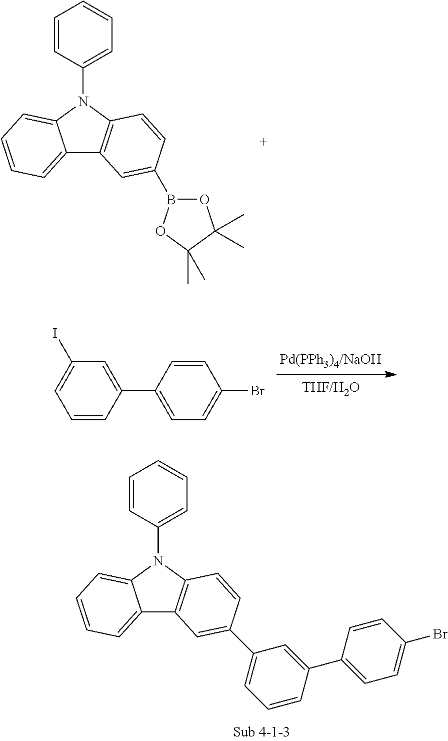

M4-2-1 (29.5 g, 80 mmol) was dissolved in THF 360 mL, and 4′-bromo-3-iodo-1,1′-biphenyl (30.16 g, 84 mmol), Pd(PPh₃)₄ (2.8 g, 2.4 mmol), NaOH (9.6 g, 240 mmol), and water (180 mL) were added and refluxed with stirring. When the reaction was completed, the reaction mixture was extracted with ether and water. The organic layer was dried over MgSO₄ and concentrated. The resulting compound was separated by silicagel column chromatography and recrystallized to obtain 24.7 g (yield: 65%) of the product.

6) Synthesis Example of Sub 4-1-4

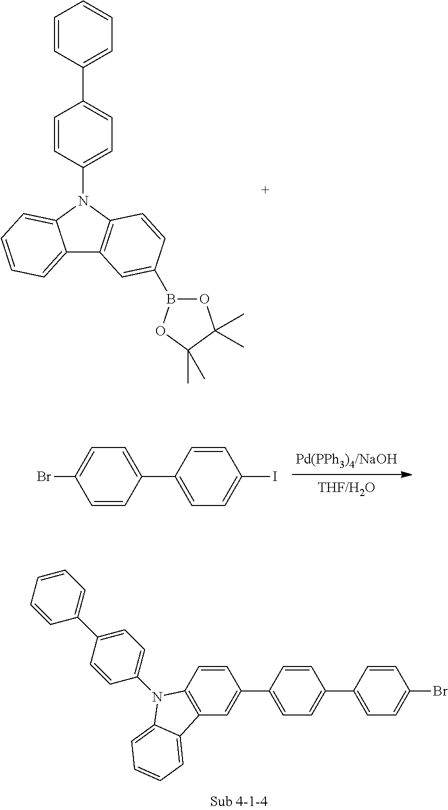 [SMILES: CC1(C)OB(c2ccc3c(c2)c2ccccc2n3-c2ccc(-c3ccccc3)cc2)OC1(C)C ; Brc1ccc(-c2ccc(I)cc2)cc1 ; Brc1ccc(-c2ccc(-c3ccc4c(c3)c3ccccc3n4-c3ccc(-c4ccccc4)cc3)cc2)cc1]

M4-2-2 (35.63 g, 80 mmol) was dissolved in THF 360 mL, 4-bromo-4′-iodo-1,1′-biphenyl (30.16 g, 84 mmol), Pd(PPh₃)₄ (2.8 g, 2.4 mmol), NaOH (9.6 g, 240 mmol), and water (180 mL) were added and refluxed with stirring. When the reaction was completed, the reaction mixture was extracted with ether and water. The organic layer was dried over MgSO₄ and concentrated. The resulting compound was separated by silicagel column chromatography and recrystallized to obtain 29.51 g (yield: 67%) of the product.

3. Synthesis Example of Sub 5

Sub 5 of Reaction Scheme 4 is the same as the synthesis example of Sub 2 of Reaction Scheme 3 below.

Synthesis of Final Product of Formula (2)

Synthesis Example of 5-17

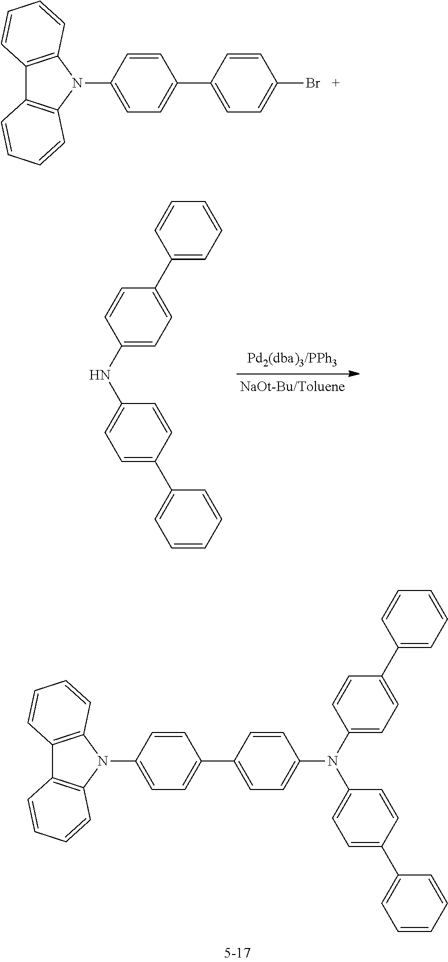 [SMILES: Brc1ccc(-c2ccc(-n3c4ccccc4c4ccccc43)cc2)cc1 ; c1ccc(-c2ccc(Nc3ccc(-c4ccccc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

9-(4′-bromo-[1,1′-biphenyl]-4-yl)-9H-carbazole(9.6 g, 24 mmol) was dissolved in toluene, and di([1,1′-biphenyl]-4-yl)amine(6.4 g, 20 mmol), Pd₂(dba)₃ (0.05 eq.), PPh₃ (0.1 eq.), NaOt-Bu (3 eq.) were added and refluxed with stirring at 100° C. for 24 hours. When the reaction was completed, the reaction mixture was extracted with ether and water. The organic layer was dried over MgSO₄ and concentrated. The resulting compound was separated by silicagel column chromatography and recrystallized to obtain 12.9 g (yield: 84%) of the product.

Synthesis Example of 5-32

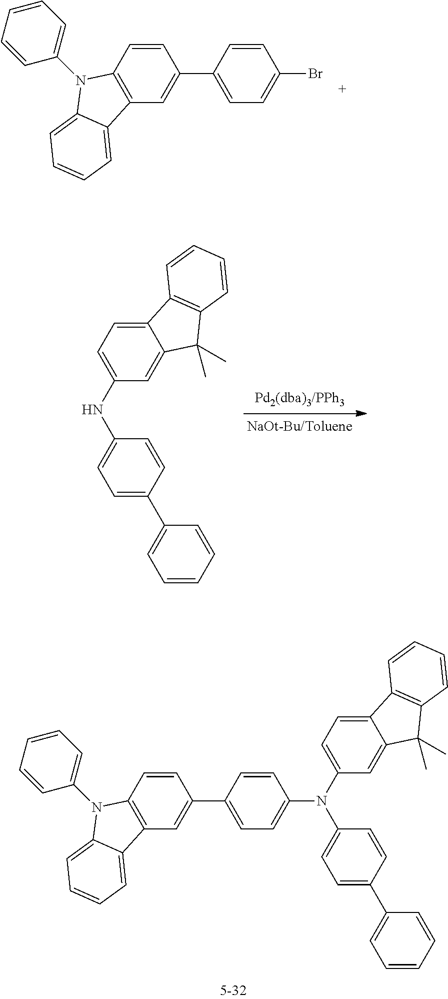 [SMILES: Brc1ccc(-c2ccc3c(c2)c2ccccc2n3-c2ccccc2)cc1 ; CC1(C)c2ccccc2-c2ccc(Nc3ccc(-c4ccccc4)cc3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

3-(4-bromophenyl)-9-phenyl-9H-carbazole (9.6 g, 24 mmol) was dissolved in toluene, and N-([1,1′-biphenyl]-4-yl)-9,9-dimethyl-9H-fluoren-2-amine (7.2 g, 20 mmol), Pd₂(dba)₃ (0.05 eq.), PPh₃ (0.1 eq.), NaOt-Bu (3 eq.) were added and refluxed with stirring at 100° C. for 24 hours. When the reaction was completed, the reaction mixture was extracted with ether and water. The organic layer was dried over MgSO₄ and concentrated. The resulting compound was separated by silicagel column chromatography and recrystallized to obtain 13.8 g (yield: 85%) of the product.

Synthesis Example of 5-61

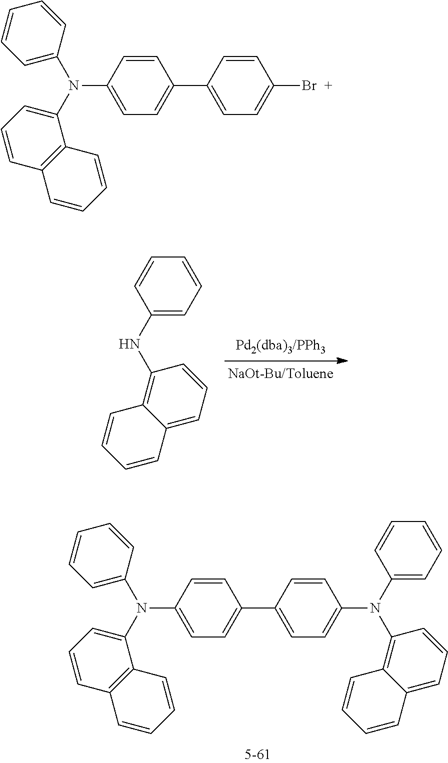 [SMILES: Brc1ccc(-c2ccc(N(c3ccccc3)c3cccc4ccccc34)cc2)cc1 ; c1ccc(Nc2cccc3ccccc23)cc1 ; c1ccc(N(c2ccc(-c3ccc(N(c4ccccc4)c4cccc5ccccc45)cc3)cc2)c2cccc3ccccc23)cc1]

N-(4′-bromo-[1,1′-biphenyl]-4-yl)-N-phenylnaphthalen-1-amine (10.8 g, 24 mmol) was dissolved in toluene and, N-phenylnaphthalen-1-amine (4.4 g, 20 mmol), Pd₂(dba)₃ (0.05 eq.), PPh₃ (0.1 eq.), NaOt-Bu (3 eq.) were added and refluxed with stirring at 100° C. for 24 hours. When the reaction was completed, the reaction mixture was extracted with ether and water. The organic layer was dried over MgSO₄ and concentrated. The resulting compound was separated by silicagel column chromatography and recrystallized to obtain 11.4 g (yield: 81%) of the product.

* Some of the products obtained above were confirmed by Mass Data as follows.

TABLE 4

| compound | FD-MS | compound | FD-MS |
|---|---|---|---|
| 5-17 | m/z = 638.27 (C₄₈H₃₄N₂ = 638.80) | 5-20 | m/z = 678.30 (C₅₁H₃₈N₂ = 678.86) |
| 5-21 | m/z = 802.33 (C₆₁H₄₂N₂ = 803.00) | 5-22 | m/z = 800.32 (C₆₁H₄₀N₂ = 800.98) |
| 5-32 | m/z = 678.30 (C₅₁H₃₈N₂ = 678.86) | 5-33 | m/z = 802.33 (C₆₁H₄₂N₂ = 803.00) |
| 5-34 | m/z = 800.32 (C₆₁H₄₀N₂ = 800.98) | 5-43 | m/z = 714.30 (C₅₄H₃₈N₂ = 714.89) |
| 5-44 | m/z = 754.33 (C₅₇H₄₂N₂ = 754.96) | 5-45 | m/z = 878.37 (C₆₇H₄₆N₂ = 879.10) |
| 5-46 | m/z = 876.35 (C₆₇H₄₄N₂ = 877.08) | 5-47 | m/z = 744.26 (C₅₄H₃₆N₂S = 744.94) |
| 5-52 | m/z = 826.33 (C₆₃H₄₂N₂ = 827.02) | 5-53 | m/z = 824.32 (C₆₃H₄₀N₂ = 825.01) |
| 5-54 | m/z = 688.29 (C₅₂H₃₆N₂ = 688.86) | 5-55 | m/z = 728.32 (C₅₅H₄₀N₂ = 728.92) |
| 5-57 | m/z = 778.33 (C₅₉H₄₂N₂ = 778.98) | 5-58 | m/z = 902.37 (C₆₉H₄₆N₂ = 903.12) |
| 5-59 | m/z = 900.35 (C₆₉H₄₄N₂ = 901.10) | 5-60 | m/z = 538.24 (C₄₀H₃₀N₂ = 538.68) |
| 5-61 | m/z = 588.26 (C₄₄H₃₂N₂ = 588.74) | 5-62 | m/z = 588.26 (C₄₄H₃₂N₂ = 588.74) |
| 5-63 | m/z = 614.27 (C₄₆H₃₄N₂ = 614.78) | | |

Manufacture and evaluation of organic electric element

### EXAMPLE 1

### Manufacture and Testing of a Green Organic Light Emitting Diode

First, on an ITO layer(anode) formed on a glass substrate, N¹-(naphthalen-2-yl)-N⁴,N⁴-bis(4-(naphthalen-2-yl(phenyl)amino)phenyl)-N¹-phenyl benzene-1,4-diamine(hereinafter will be abbreviated as 2-TNATA) was vacuum-deposited to form a hole injection layer with a thickness of 60 nm. Subsequently, 4,4-bis [N-(1-naphthyl)-N-phenylamino] biphenyl (hereinafter will be abbreviated as -NPD) as a hole transport compound was vapor-deposited on the film to a thickness of 60 nm to form a hole transport layer. Subsequently, the inventive compounds and examples were vacuum-deposited as an emitting-auxiliary layer material to a thickness of 20 nm to form an emitting-auxiliary layer. After forming the emitting-auxiliary layer, CBP [4,4′-N, N′-dicarbazole-biphenyl] as a host was used in the upper of an emitting auxiliary layer,and Ir(ppy)3 [tris(2-phenylpyridine)-iridium] as a dopant was vacuum deposited to form an emitting layer with a thickness of 30 nm by doping with a weight ratio of 95:5. (1,1′-bisphenyl)-4-olato)bis(2-methyl-8-quinolinolato)aluminum (hereinafter will be abbreviated as BAlq) was vacuum-deposited to a thickness of 10 nm as a hole blocking layer, and tris(8-quinolinol)aluminum(hereinafter will be abbreviated as Alq3) was deposited to a thickness of 40 nm as an electron transport layer. After that, an alkali metal halide, LiF was vacuum deposited as an electron injection layer to a thickness of 0.2 nm, and Al was deposited to a thickness of 150 nm to form a cathode to manufacture an OLED.

To the OLEDs which were manufactured by examples and comparative examples, a forward bias direct current voltage was applied, and electroluminescent (EL) properties were measured using PR-650 of Photoresearch Co., and T95 life was measured using a life measuring apparatus manufactured by McScience Inc. with a reference luminance of 5000 cd/m². In the following table, the manufacture of a device and the results of evaluation are shown.

[Comparative 1]
An organic electroluminescent device was manufactured in the same manner as in Example 1, except that the emitting-auxiliary layer was not used.

[Comparative 2]
An organic electroluminescent device was manufactured in the same manner as in Example 1, except that the emitting-auxiliary layer was formed using the Comparative Example 1 instead of the compound of the present invention.

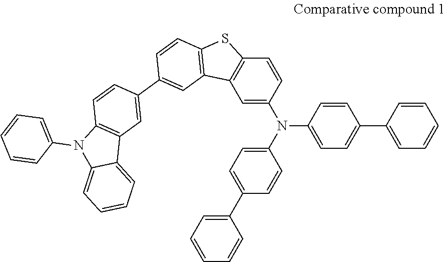 [SMILES: c1ccc(-c2ccc(N(c3ccc(-c4ccccc4)cc3)c3ccc4sc5ccc(-c6ccc7c(c6)c6ccccc6n7-c6ccccc6)cc5c4c3)cc2)cc1]

TABLE 5

| | | | | Bright- | Effi- | Life- | CIE |
|---|---|---|---|---|---|---|---|
| | | Volt- | Current | ness | cien- | time | (x, |
| | compound | age | Density | (cd/m2) | cy | T(95) | y) |
| comparative | — | 6.0 | 21.7 | 5000.0 | 23.0 | 61.8 | (0.31, |
| example(1) | | | | | | | 0.61) |
| comparative | Com- | 6.2 | 14.3 | 5000.0 | 35.0 | 94..5 | (0.31, |
| | parative | | | | | | 0.60) |
| example(2) | com- | | | | | | |
| | pound 1 | | | | | | |
| example(1) | Compound | 5.6 | 10.9 | 5000.0 | 45.8 | 118.1 | (0.31, |
| | (1-1) | | | | | | 0.60) |
| example(2) | Compound | 5.5 | 11.3 | 5000.0 | 44.3 | 114.2 | (0.32, |
| | (1-2) | | | | | | 0.61) |
| example(3) | Compound | 5.6 | 11.2 | 5000.0 | 44.7 | 117.7 | (0.31, |
| | (1-3) | | | | | | 0.61) |
| example(4) | com- | 5.7 | 10.7 | 5000.0 | 46.7 | 114.1 | (0.33, |
| | pound(1-4) | | | | | | 0.60) |
| example(5) | Compound | 5.7 | 10.8 | 5000.0 | 46.4 | 113.6 | (0.31, |
| | (1-6) | | | | | | 0.60) |
| example(6) | Compound | 5.5 | 11.5 | 5000.0 | 43.4 | 119.9 | (0.32, |
| | (1-7) | | | | | | 0.61) |
| example(7) | Compound | 5.5 | 11.5 | 5000.0 | 43.5 | 113.8 | (0.32, |
| | (1-8) | | | | | | 0.61) |
| example(8) | Compound | 5.6 | 11.6 | 5000.0 | 43.1 | 116.6 | (0.33, |
| | (1-13) | | | | | | 0.60) |
| example(9) | Compound | 5.6 | 11.1 | 5000.0 | 45.1 | 119.0 | (0.30, |
| | (1-14) | | | | | | 0.61) |
| example(10) | Compound | 5.7 | 10.6 | 5000.0 | 47.1 | 119.2 | (0.31, |
| | (1-15) | | | | | | 0.61) |
| example(11) | Compound | 5.6 | 10.5 | 5000.0 | 47.5 | 118.1 | (0.30, |
| | (2-1) | | | | | | 0.60) |
| example(12) | Compound | 5.5 | 11.8 | 5000.0 | 42.2 | 116.0 | (0.33, |
| | (2-2) | | | | | | 0.61) |
| example(13) | Compound | 5.5 | 11.9 | 5000.0 | 42.0 | 113.8 | (0.32, |
| | (2-3) | | | | | | 0.61) |
| example(14) | Compound | 5.6 | 11.0 | 5000.0 | 45.5 | 118.3 | (0.33, |
| | (2-4) | | | | | | 0.60) |
| example(15) | Compound | 5.6 | 10.8 | 5000.0 | 46.4 | 111.9 | (0.30, |
| | (2-6) | | | | | | 0.61) |
| example(16) | Compound | 5.6 | 11.4 | 5000.0 | 43.7 | 114.6 | (0.31, |
| | (2-7) | | | | | | 0.61) |
| example(17) | Compound | 5.6 | 11.7 | 5000.0 | 42.7 | 117.5 | (0.31, |
| | (2-8) | | | | | | 0.60) |
| example(18) | Compound | 5.6 | 11.4 | 5000.0 | 43.7 | 119.8 | (0.33, |
| | (2-13) | | | | | | 0.61) |
| example(19) | Compound | 5.7 | 10.9 | 5000.0 | 45.9 | 117.5 | (0.32, |
| | (2-14) | | | | | | 0.60) |
| example(20) | Compound | 5.7 | 10.7 | 5000.0 | 46.7 | 114.0 | (0.32, |
| | (2-15) | | | | | | 0.61) |

As it is apparent from the results of Table 5, when a green organic electroluminescent device is manufactured using the material for an organic electric element of the present invention as an emitting-auxiliary layer material, not only the driving voltage of the organic electric element can be lowered, but also the luminous efficiency and life span can be remarkably improved as compared with the comparative examples not using the emitting-auxiliary layer or using the comparative compound 1.

That is, the results of Comparative Example 2 using the comparative compound 1 at the emitting auxiliary layer, and Examples 1 to 20 using the compound of the present invention were superior to those of Comparative Example 1 not using emitting auxiliary layer, and the compounds of the invention substituted with asymmetrically limited at positions 2 and 3 of dibenzothiophen or dibenzofuran showed the best results than the comparative compound 1 in which both carbazole and amine groups were substituted at position 2 of dibenzothiophen.

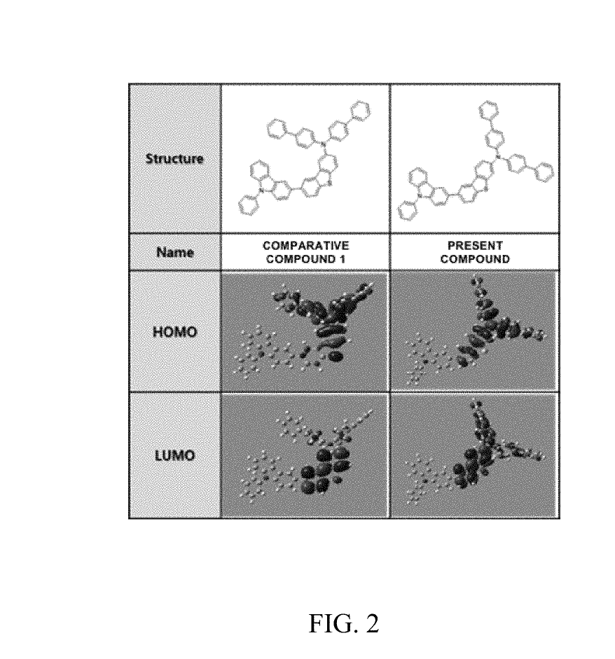

It can be confirmed that the electron cloud degree of Comparative Compound 1 and the inventive compound are different from each other as shown in FIG. 2, and a large difference can be confirmed especially in LUMO. That is, the electronic cloud degree of LUMO shows that the electron cloud degree of Comparative Compound 1 is concentrated in dibenzothiophen, but the inventive compound has electron clouds formed to both dibenzothiophen and amine groups.

Therefore, as described above, it can be confirmed that the physical properties of the compound are changed depending on the position of the substituent, even though they are the same core and substituent, and this can act as a main factor for improving the device performance, and different results are obtained. That is, as substituents are asymmetrically substituted at positions 2 and 3 of dibenzothiophen or dibenzofuran, the physical properties of the compound and the result of the device are significantly different.

### EXAMPLE 2

### Manufacture and Testing of a Red Organic Light Emitting Diode

First, on an ITO layer(anode) formed on a glass substrate, N¹-(naphthalen-2-yl)-N⁴,N⁴-bis(4-(naphthalen-2-yl(phenyl)amino)phenyl)-N¹-phenyl benzene-1,4-diamine(hereinafter will be abbreviated as 2-TNATA) was vacuum-deposited to form a hole injection layer with a thickness of 60 nm.

Subsequently, the inventive compound represented by Formula (13) as a hole transport compound was vapor-deposited on the film to a thickness of 60 nm to form a hole transport layer. Subsequently, the inventive compounds represented by Formula (1) were vacuum-deposited as an emitting-auxiliary layer material to a thickness of 20 nm to form an emitting-auxiliary layer. After forming the emitting-auxiliary layer, CBP [4,4′-N, N′-dicarbazole-biphenyl] as a host was used in the upper of the emitting layer, and (piq)₂Ir(acac) [bis-(1-phenylisoquinolyl)iridium(III)acetylacetonate]as a dopant was vacuum deposited to form an emitting layer with a thickness of 30 nm on the emitting auxiliary layer by doping with a weight ratio of 95:5. (1,1′-bisphenyl)-4-olato)bis(2-methyl-8-quinolinolato)aluminum (hereinafter will be abbreviated as BAlq) was vacuum-deposited to a thickness of 10 nm as a hole blocking layer, and tris(8-quinolinol)aluminum(hereinafter will be abbreviated as Alq3) was deposited to a thickness of 40 nm as an electron transport layer. After that, an alkali metal halide, LiF was vacuum deposited as an electron injection layer to a thickness of 0.2 nm, and Al was deposited to a thickness of 150 nm to form a cathode to manufacture an OLED.

To the OLEDs which were manufactured by examples and comparative examples, a forward bias direct current voltage was applied, and electroluminescent (EL) properties were measured using PR-650 of Photoresearch Co., and T95 life was measured using a life measuring apparatus manufactured by McScience Inc. with a reference luminance of 2500 cd/m². In the following table, the manufacture of a device and the results of evaluation are shown.

[Comparative 3, 4, 5]

An organic electroluminescent device was fabricated in the same manner as in Example 2, except that Comparative Compound 1 was used as the emitting-auxiliary layer.

TABLE 7

| | Hole | Emitting | | | | | |
|---|---|---|---|---|---|---|---|
| | transport | auxiliary | | Current | Brightness | | Lifetime |
| | compound | compound | Voltage | Density | (cd/m2) | Efficiency | T(95) |
| comparative | Compound | Comparative | 5.7 | 27.2 | 2500.0 | 9.2 | 85.3 |
| example(3) | (5-17) | compound 1 | | | | | |
| comparative | Compound | Comparative | 5.2 | 23.1 | 2500.0 | 10.8 | 87.7 |
| example(4) | (5-32) | compound 1 | | | | | |
| comparative | Compound | Comparative | 5.4 | 25.5 | 2500.0 | 9.8 | 82.9 |
| example(5) | (5-61) | compound 1 | | | | | |
| example(21) | Compound | compound(1- | 5.1 | 20.7 | 2500.0 | 12.1 | 108.4 |
| | (5-17) | 1) | | | | | |
| example(22) | Compound | compound(1- | 5.3 | 24.1 | 2500.0 | 10.4 | 106.5 |
| | (5-17) | 2) | | | | | |
| example(23) | Compound | compound(1- | 5.4 | 21.8 | 2500.0 | 11.5 | 106.8 |
| | (5-17) | 3) | | | | | |
| example(24) | Compound | compound(1- | 5.1 | 19.2 | 2500.0 | 13.0 | 112.2 |
| | (5-17) | 4) | | | | | |
| example(25) | Compound | compound(1- | 5.2 | 18.2 | 2500.0 | 13.7 | 101.8 |
| | (5-17) | 6) | | | | | |
| example(26) | Compound | compound(1- | 5.4 | 24.7 | 2500.0 | 10.1 | 103.7 |
| | (5-17) | 7) | | | | | |
| example(27) | Compound | compound(1- | 5.4 | 23.7 | 2500.0 | 10.5 | 109.9 |
| | (5-17) | 8) | | | | | |
| example(28) | Compound | compound(1- | 5.4 | 21.1 | 2500.0 | 11.8 | 102.8 |
| | (5-17) | 13) | | | | | |
| example(29) | Compound | compound(1- | 5.2 | 19.1 | 2500.0 | 13.1 | 106.9 |
| | (5-17) | 14) | | | | | |
| example(30) | Compound | compound(1- | 5.1 | 17.9 | 2500.0 | 14.0 | 113.2 |
| | (5-17) | 15) | | | | | |
| example(31) | Compound | compound(2- | 5.1 | 18.3 | 2500.0 | 13.7 | 118.3 |
| | (5-17) | 1) | | | | | |
| example(32) | Compound | compound(2- | 5.4 | 22.2 | 2500.0 | 11.2 | 109.7 |
| | (5-17) | 2) | | | | | |
| example(33) | Compound | compound(2- | 5.3 | 23.4 | 2500.0 | 10.7 | 101.5 |
| | (5-17) | 3) | | | | | |
| example(34) | Compound | compound(2- | 5.1 | 18.7 | 2500.0 | 13.4 | 114.1 |
| | (5-17) | 4) | | | | | |
| example(35) | Compound | compound(2- | 5.2 | 18.1 | 2500.0 | 13.8 | 116.7 |
| | (5-17) | 6) | | | | | |
| example(36) | Compound | compound(2- | 5.5 | 24.7 | 2500.0 | 10.1 | 101.4 |
| | (5-17) | 7) | | | | | |
| example(37) | Compound | compound(2- | 5.4 | 24.2 | 2500.0 | 10.3 | 109.2 |
| | (5-17) | 8) | | | | | |
| example(38) | Compound | compound(2- | 5.3 | 21.5 | 2500.0 | 11.6 | 114.1 |
| | (5-17) | 13) | | | | | |
| example(39) | Compound | compound(2- | 5.3 | 18.1 | 2500.0 | 13.8 | 117.1 |
| | (5-17) | 14) | | | | | |
| example(40) | Compound | compound(2- | 5.2 | 18.8 | 2500.0 | 13.3 | 103.7 |
| | (5-17) | 15) | | | | | |
| example(41) | compound( | compound(1- | 4.5 | 15.1 | 2500.0 | 16.5 | 113.4 |
| | 5-32) | 1) | | | | | |
| example(42) | Compound | compound(1- | 5.0 | 19.1 | 2500.0 | 13.1 | 102.7 |
| | (5-32) | 2) | | | | | |
| example(43) | Compound | compound(1- | 5.0 | 17.2 | 2500.0 | 14.6 | 116.7 |
| | (5-32) | 3) | | | | | |
| example(44) | Compound | compound(1- | 4.8 | 14.9 | 2500.0 | 16.8 | 106.6 |
| | (5-32) | 4) | | | | | |
| example(45) | Compound | compound(1- | 4.8 | 15.1 | 2500.0 | 16.5 | 110.0 |
| | (5-32) | 6) | | | | | |
| example(46) | Compound | compound(1- | 4.9 | 18.3 | 2500.0 | 13.7 | 101.2 |
| | (5-32) | 7) | | | | | |
| example(47) | Compound | compound(1- | 5.0 | 16.9 | 2500.0 | 14.8 | 116.7 |
| | (5-32) | 8) | | | | | |
| example(48) | Compound | compound(1- | 5.0 | 18.0 | 2500.0 | 13.9 | 116.0 |
| | (5-32) | 13) | | | | | |
| example(49) | Compound | compound(1- | 4.6 | 15.7 | 2500.0 | 15.9 | 114.8 |
| | (5-32) | 14) | | | | | |
| example(50) | Compound | compound(1- | 4.7 | 14.9 | 2500.0 | 16.7 | 102.2 |
| | (5-32) | 15) | | | | | |
| example(51) | Compound | compound(2- | 4.5 | 15.2 | 2500.0 | 16.5 | 105.1 |
| | (5-32) | 1) | | | | | |
| example(52) | Compound | compound(2- | 5.0 | 18.1 | 2500.0 | 13.8 | 114.3 |
| | (5-32) | 2) | | | | | |
| example(53) | Compound | compound(2- | 4.9 | 18.1 | 2500.0 | 13.8 | 115.5 |
| | (5-32) | 3) | | | | | |
| example(54) | Compound | compound(2- | 4.8 | 15.8 | 2500.0 | 15.9 | 110.3 |
| | (5-32) | 4) | | | | | |
| example(55) | Compound | compound(2- | 4.6 | 15.1 | 2500.0 | 16.5 | 118.4 |
| | (5-32) | 6) | | | | | |
| example(56) | Compound | compound(2- | 5.0 | 17.7 | 2500.0 | 14.1 | 106.5 |
| | (5-32) | 7) | | | | | |
| example(57) | Compound | compound(2- | 4.9 | 19.2 | 2500.0 | 13.1 | 109.4 |
| | (5-32) | 8) | | | | | |
| example(58) | Compound | compound(2- | 4.9 | 19.2 | 2500.0 | 13.0 | 100.9 |
| | (5-32) | 13) | | | | | |
| example(59) | Compound | compound(2- | 4.6 | 15.7 | 2500.0 | 15.9 | 107.1 |
| | (5-32) | 14) | | | | | |
| example(60) | Compound | compound(2- | 4.8 | 15.5 | 2500.0 | 16.1 | 109.1 |
| | (5-32) | 15) | | | | | |
| example(61) | Compound | compound(1- | 4.8 | 17.1 | 2500.0 | 14.6 | 113.4 |
| | (5-61) | 1) | | | | | |
| example(62) | Compound | compound(1- | 5.0 | 20.1 | 2500.0 | 12.4 | 115.8 |
| | (5-61) | 2) | | | | | |
| example(63) | Compound | compound(1- | 5.1 | 19.7 | 2500.0 | 12.7 | 103.3 |
| | (5-61) | 3) | | | | | |
| example(64) | Compound | compound(1- | 4.8 | 16.8 | 2500.0 | 14.9 | 100.7 |
| | (5-61) | 4) | | | | | |
| example(65) | Compound | compound(1- | 4.8 | 17.0 | 2500.0 | 14.7 | 109.0 |
| | (5-61) | 6) | | | | | |
| example(66) | Compound | compound(1- | 4.9 | 20.6 | 2500.0 | 12.1 | 110.0 |
| | (5-61) | 7) | | | | | |
| example(67) | Compound | compound(1- | 5.0 | 19.7 | 2500.0 | 12.7 | 100.1 |
| | (5-61) | 8) | | | | | |
| example(68) | Compound | compound(1- | 5.1 | 18.1 | 2500.0 | 13.8 | 117.9 |
| | (5-61) | 13) | | | | | |
| example(69) | Compound | compound(1- | 4.6 | 16.3 | 2500.0 | 15.4 | 104.8 |
| | (5-61) | 14) | | | | | |
| example(70) | Compound | compound(1- | 4.6 | 16.2 | 2500.0 | 15.5 | 110.9 |
| | (5-61) | 15) | | | | | |
| example(71) | Compound | compound(2- | 4.6 | 17.3 | 2500.0 | 14.5 | 111.2 |
| | (5-61) | 1) | | | | | |
| example(72) | Compound | compound(2- | 5.0 | 20.1 | 2500.0 | 12.5 | 117.4 |
| | (5-61) | 2) | | | | | |
| example(73) | Compound | compound(2- | 4.9 | 18.9 | 2500.0 | 13.2 | 116.9 |
| | (5-61) | 3) | | | | | |
| example(74) | Compound | compound(2- | 4.8 | 17.0 | 2500.0 | 14.7 | 119.6 |
| | (5-61) | 4) | | | | | |
| example(75) | Compound | compound(2- | 4.6 | 17.3 | 2500.0 | 14.4 | 107.7 |
| | (5-61) | 6) | | | | | |
| example(76) | Compound | compound(2- | 5.1 | 17.9 | 2500.0 | 14.0 | 107.6 |
| | (5-61) | 7) | | | | | |
| example(77) | Compound | compound(2- | 4.9 | 20.8 | 2500.0 | 12.0 | 115.2 |
| | (5-61) | 8) | | | | | |
| example(78) | Compound | compound(2- | 5.0 | 18.1 | 2500.0 | 13.8 | 103.8 |
| | (5-61) | 13) | | | | | |
| example(79) | Compound | compound(2- | 4.7 | 17.3 | 2500.0 | 14.5 | 102.0 |
| | (5-61) | 14) | | | | | |
| example(80) | Compound | compound(2- | 4.8 | 15.7 | 2500.0 | 16.0 | 104.7 |
| | (5-61) | 15) | | | | | |

As can be seen from the results of Table 7, when the compound represented by the Formula (5) was used as a hole transport layer and the compound represented by the Formula (1) was used as an emitting auxiliary layer, the driving voltage and lifetime, particularly the luminous efficiency, of the organic electroluminescent device are remarkably improved as compared with the comparative example using the comparative compound 1 as the emitting auxiliary layer, although other conditions are the same. This is because when the physical and chemical properties of the inventive compound represented by Formula 1 described in Table 5 together with the compound represented by Formula 5 are constituted together with the compound, the combination has electrochemical synergistic action to improve the performance of the entire device.

Although exemplary embodiments of the present invention have been described for illustrative purposes, those skilled in the art will appreciate that various modifications, additions and substitutions are possible, without departing from the scope and spirit of the invention as disclosed in the accompanying claims. Therefore, the embodiment disclosed in the present invention is intended to illustrate the scope of the technical idea of the present invention, and the scope of the present invention is not limited by the embodiment. The scope of the present invention shall be construed on the basis of the accompanying claims, and it shall be construed that all of the technical ideas comprised within the scope equivalent to the claims belong to the present invention.

1. A compound represented by Formula (1) below: 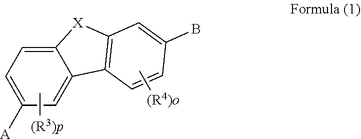
wherein,
1) A and B are each selected from the group consisting of Formula (1-1 and (1-2) and they are different, 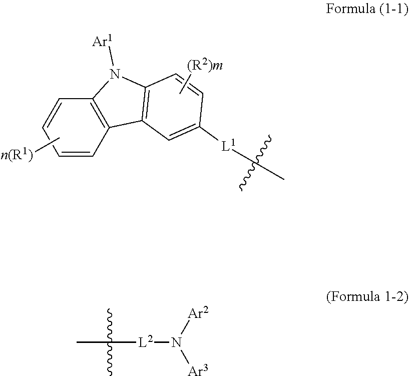
2) Ar¹, Ar² and Ar³ are each independently selected from the group consisting of a C₆-C₆₀ aryl group; a fluorenyl group; a C₂-C₆₀ heterocyclic group including at least one hetero atom of O, N, S, Si and P; a fused ring group of a C₃-C₆₀ aliphatic ring and a C₆-C₆₀ aromatic ring;
3) X is O or S,
4) L¹ and L² are independently selected from the group consisting of a single bond, a C₆-C₆₀ arylene group, and a fluorenylene group; a fused ring group of a C₃-C₆₀ aliphatic ring and a C₆-C₆₀ aromatic ring; and a C₂-C₆₀ heterocyclic group,
5) n is an integer of 0 to 4, and m, p and o are an integer of 0 to 3, when m, n, o or p are 1 or more, R¹, R², R³, and R⁴ are each independently selected from a hydrogen; a deuterium; a halogen; the group consisting of a C₆-C₆₀ aryl group; a fluorenyl group; a C₂-C₆₀ heterocyclic group including at least one hetero atom of O, N, S, Si and P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; and -L′-N(Rₐ)(R_{b})(wherein, L′ is selected from the group consisting of a single bond; a C₆-C₆₀ arylene group; a fluorenylene group; a fused ring group of a C₃-C₆₀ aliphatic ring and a C₆-C₆₀ aromatic ring; and a C₂-C₆₀ heterocyclic, and Rₐ and R_{b} are be independently selected from the group consisting of a C₆-C₆₀ aryl group; a fluorenyl group; a fused ring group of a C₃-C₆₀ aliphatic ring and a C₆-C₆₀ aromatic ring; and a C₂-C₆₀ heterocyclic group containing at least one hetero atom of O, N, S, Si, and P), or an adjacent plurality of R¹ or a plurality of R² or a plurality of R³ or a plurality of R⁴ may combine to each other to form an aromatic or a heteroaromatic ring,
wherein, the aryl group, fluorenyl group, arylene group, heterocyclic group, fused ring group, alkyl group, alkenyl group, alkoxy group and aryloxy group may be substituted with one or more substituents selected from the group consisting of deuterium; halogen; a silane group substituted or unsubstituted with C₁-C₂₀ alkyl group or C₆-C₂₀ aryl group; siloxane group; boron group; germanium group; cyano group; nitro group; -L′-N(Rₐ)(R_{b}); a C₁-C₂₀ alkylthio group; C₁-C₂₀ alkoxyl group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂-C₂₀ heterocyclic group; C₃-C₂₀ cycloalkyl group; C₇-C₂₀ arylalkyl group and C₈-C₂₀ arylalkenyl group, and also, these substituents may combine each other and form a saturated or unsaturated ring, wherein the term ‘ring’ means a C₃-C₆₀ aliphatic ring ora C₆-C₆₀ aromatic ring or a C₂-C₆₀ heterocyclic ring or a fused ring formed by the combination of thereof.

2. The compound according to claim 1, wherein the compound represented by Formula (1) is represented by Formulas (2) or (3): 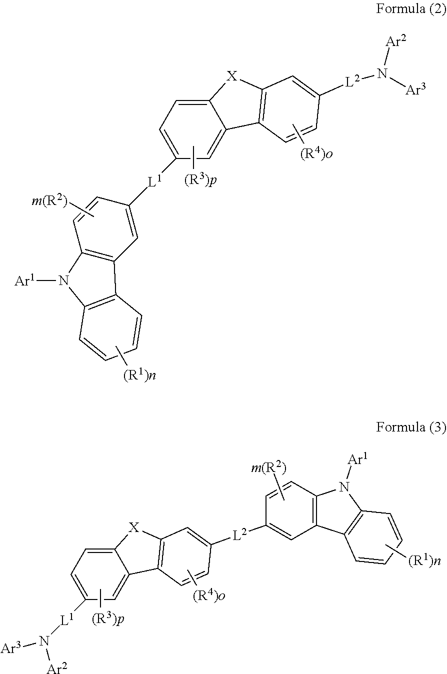
wherein
R¹, R², R³, R⁴, m, n, o, p, L¹, L², Ar¹, Ar², and Ar³ are the same as defined in claim 1.

3. The compound according to claim 1, wherein the compound represented by Formula (1) is represented by any one of the following Formulas:

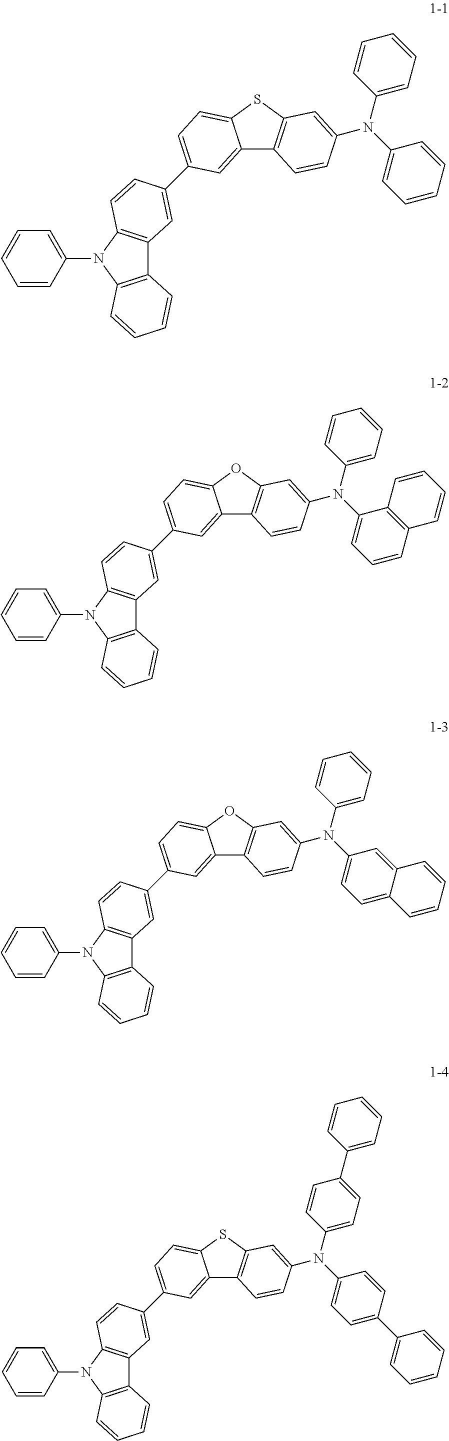 [SMILES: c1ccc(N(c2ccccc2)c2ccc3c(c2)sc2ccc(-c4ccc5c(c4)c4ccccc4n5-c4ccccc4)cc23)cc1 ; c1ccc(N(c2ccc3c(c2)oc2ccc(-c4ccc5c(c4)c4ccccc4n5-c4ccccc4)cc23)c2cccc3ccccc23)cc1 ; c1ccc(N(c2ccc3ccccc3c2)c2ccc3c(c2)oc2ccc(-c4ccc5c(c4)c4ccccc4n5-c4ccccc4)cc23)cc1 ; c1ccc(-c2ccc(N(c3ccc(-c4ccccc4)cc3)c3ccc4c(c3)sc3ccc(-c5ccc6c(c5)c5ccccc5n6-c5ccccc5)cc34)cc2)cc1]

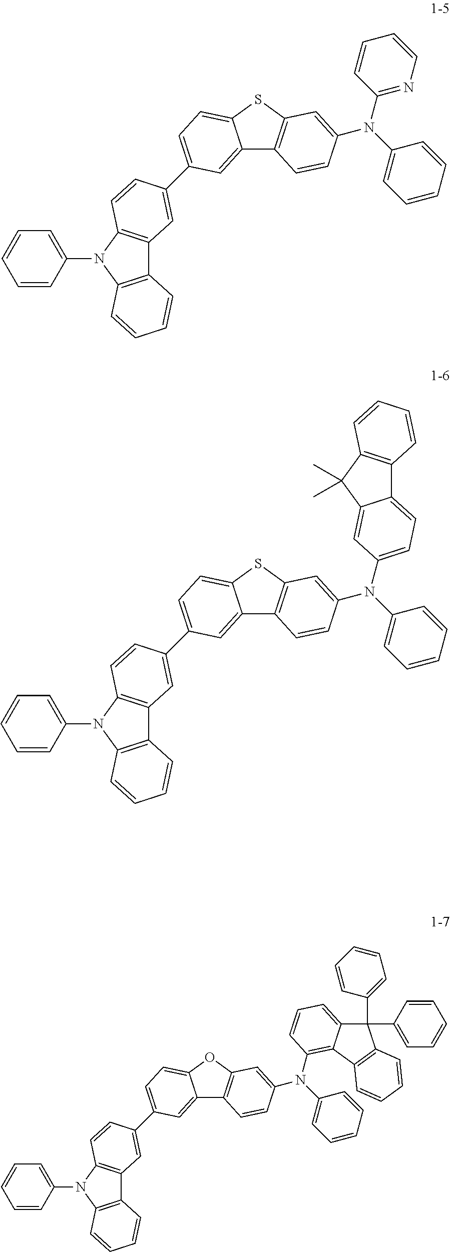 [SMILES: c1ccc(N(c2ccc3c(c2)sc2ccc(-c4ccc5c(c4)c4ccccc4n5-c4ccccc4)cc23)c2ccccn2)cc1 ; CC1(C)c2ccccc2-c2ccc(N(c3ccccc3)c3ccc4c(c3)sc3ccc(-c5ccc6c(c5)c5ccccc5n6-c5ccccc5)cc34)cc21 ; c1ccc(N(c2ccc3c(c2)oc2ccc(-c4ccc5c(c4)c4ccccc4n5-c4ccccc4)cc23)c2cccc3c2-c2ccccc2C3(c2ccccc2)c2ccccc2)cc1]

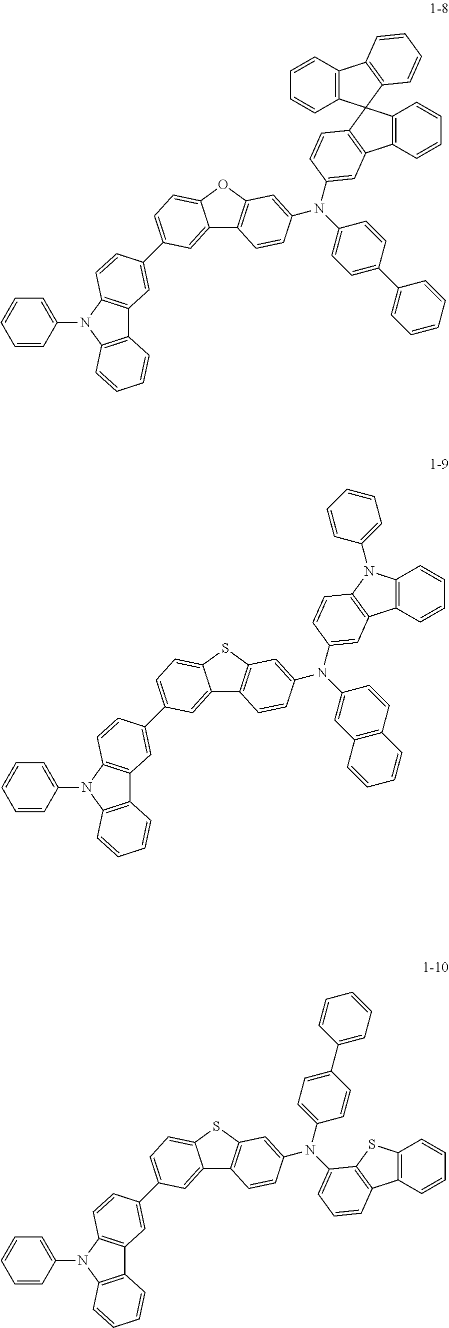 [SMILES: c1ccc(-c2ccc(N(c3ccc4c(c3)-c3ccccc3C43c4ccccc4-c4ccccc43)c3ccc4c(c3)oc3ccc(-c5ccc6c(c5)c5ccccc5n6-c5ccccc5)cc34)cc2)cc1 ; c1ccc(-n2c3ccccc3c3cc(-c4ccc5sc6cc(N(c7ccc8ccccc8c7)c7ccc8c(c7)c7ccccc7n8-c7ccccc7)ccc6c5c4)ccc32)cc1 ; c1ccc(-c2ccc(N(c3ccc4c(c3)sc3ccc(-c5ccc6c(c5)c5ccccc5n6-c5ccccc5)cc34)c3cccc4c3sc3ccccc34)cc2)cc1]

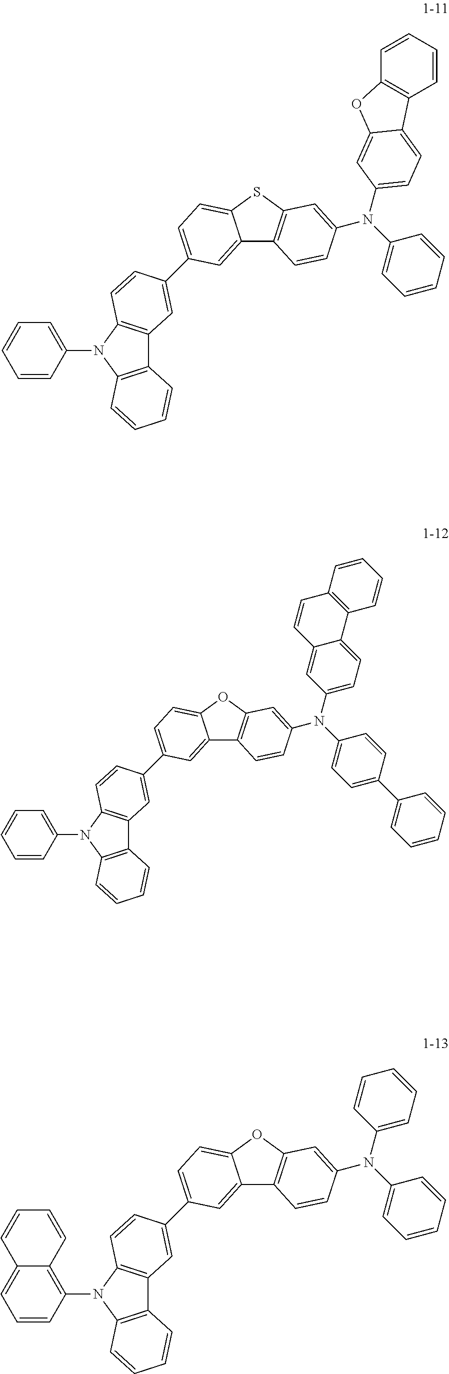 [SMILES: c1ccc(N(c2ccc3c(c2)oc2ccccc23)c2ccc3c(c2)sc2ccc(-c4ccc5c(c4)c4ccccc4n5-c4ccccc4)cc23)cc1 ; c1ccc(-c2ccc(N(c3ccc4c(ccc5ccccc54)c3)c3ccc4c(c3)oc3ccc(-c5ccc6c(c5)c5ccccc5n6-c5ccccc5)cc34)cc2)cc1 ; c1ccc(N(c2ccccc2)c2ccc3c(c2)oc2ccc(-c4ccc5c(c4)c4ccccc4n5-c4cccc5ccccc45)cc23)cc1]

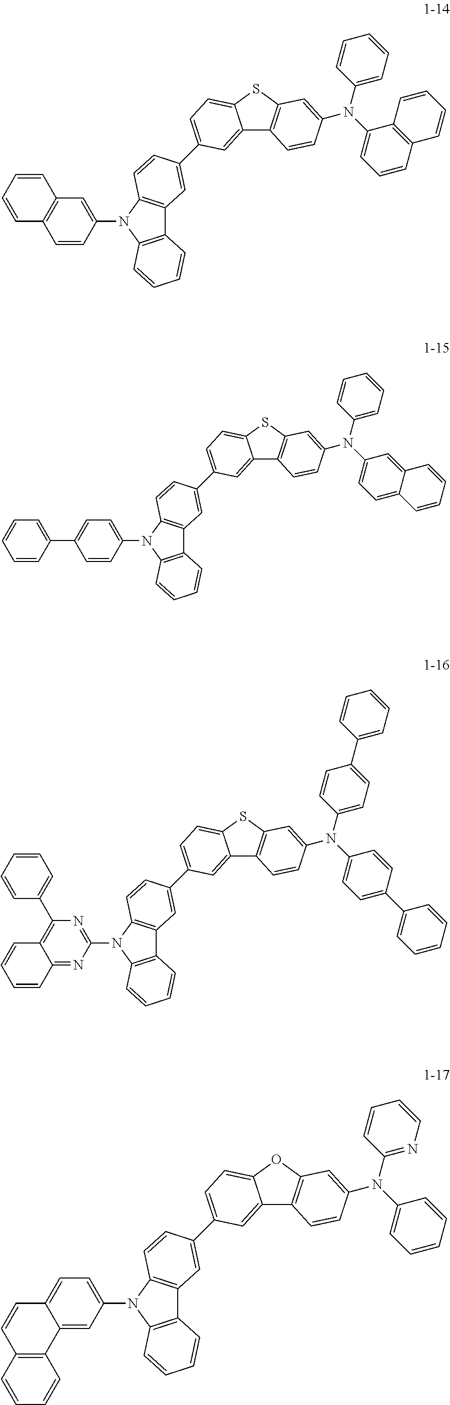 [SMILES: c1ccc(N(c2ccc3c(c2)sc2ccc(-c4ccc5c(c4)c4ccccc4n5-c4ccc5ccccc5c4)cc23)c2cccc3ccccc23)cc1 ; c1ccc(-c2ccc(-n3c4ccccc4c4cc(-c5ccc6sc7cc(N(c8ccccc8)c8ccc9ccccc9c8)ccc7c6c5)ccc43)cc2)cc1 ; c1ccc(-c2ccc(N(c3ccc(-c4ccccc4)cc3)c3ccc4c(c3)sc3ccc(-c5ccc6c(c5)c5ccccc5n6-c5nc(-c6ccccc6)c6ccccc6n5)cc34)cc2)cc1 ; c1ccc(N(c2ccc3c(c2)oc2ccc(-c4ccc5c(c4)c4ccccc4n5-c4ccc5ccc6ccccc6c5c4)cc23)c2ccccn2)cc1]

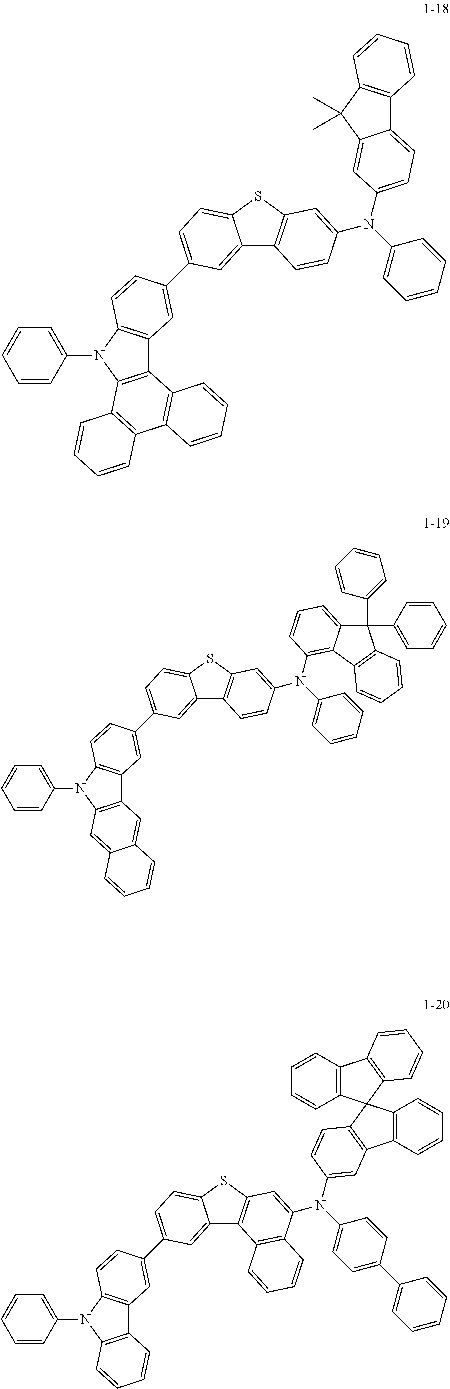 [SMILES: CC1(C)c2ccccc2-c2ccc(N(c3ccccc3)c3ccc4c(c3)sc3ccc(-c5ccc6c(c5)c5c7ccccc7c7ccccc7c5n6-c5ccccc5)cc34)cc21 ; c1ccc(N(c2ccc3c(c2)sc2ccc(-c4ccc5c(c4)c4cc6ccccc6cc4n5-c4ccccc4)cc23)c2cccc3c2-c2ccccc2C3(c2ccccc2)c2ccccc2)cc1 ; c1ccc(-c2ccc(N(c3ccc4c(c3)-c3ccccc3C43c4ccccc4-c4ccccc43)c3cc4sc5ccc(-c6ccc7c(c6)c6ccccc6n7-c6ccccc6)cc5c4c4ccccc34)cc2)cc1]

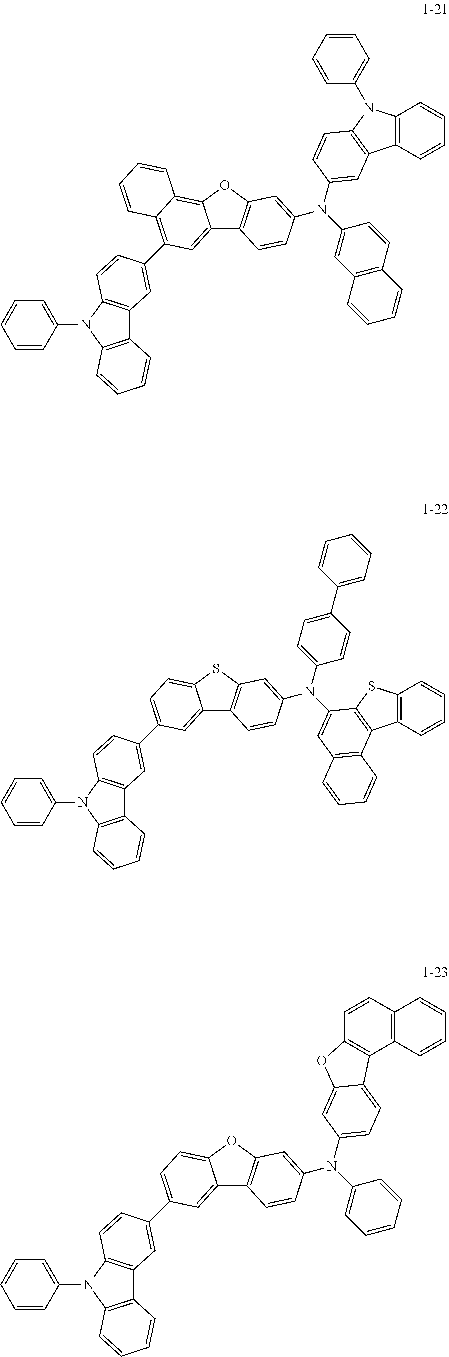 [SMILES: c1ccc(-n2c3ccccc3c3cc(-c4cc5c6ccc(N(c7ccc8ccccc8c7)c7ccc8c(c7)c7ccccc7n8-c7ccccc7)cc6oc5c5ccccc45)ccc32)cc1 ; c1ccc(-c2ccc(N(c3ccc4c(c3)sc3ccc(-c5ccc6c(c5)c5ccccc5n6-c5ccccc5)cc34)c3cc4ccccc4c4c3sc3ccccc34)cc2)cc1 ; c1ccc(N(c2ccc3c(c2)oc2ccc(-c4ccc5c(c4)c4ccccc4n5-c4ccccc4)cc23)c2ccc3c(c2)oc2ccc4ccccc4c23)cc1]

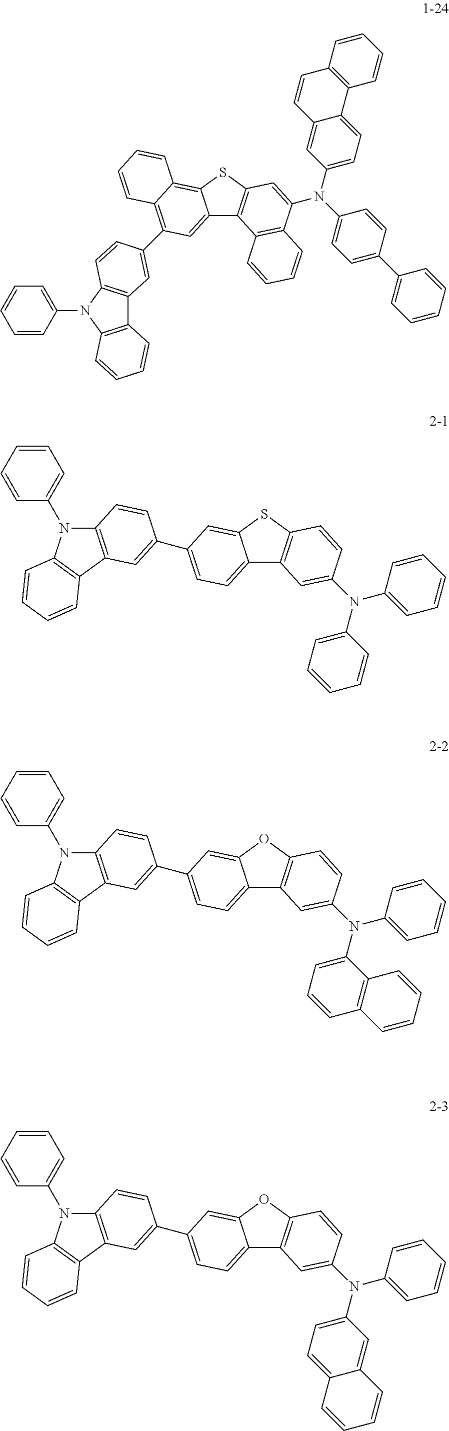 [SMILES: c1ccc(-c2ccc(N(c3ccc4c(ccc5ccccc54)c3)c3cc4sc5c6ccccc6c(-c6ccc7c(c6)c6ccccc6n7-c6ccccc6)cc5c4c4ccccc34)cc2)cc1 ; c1ccc(N(c2ccccc2)c2ccc3sc4cc(-c5ccc6c(c5)c5ccccc5n6-c5ccccc5)ccc4c3c2)cc1 ; c1ccc(N(c2ccc3oc4cc(-c5ccc6c(c5)c5ccccc5n6-c5ccccc5)ccc4c3c2)c2cccc3ccccc23)cc1 ; c1ccc(N(c2ccc3ccccc3c2)c2ccc3oc4cc(-c5ccc6c(c5)c5ccccc5n6-c5ccccc5)ccc4c3c2)cc1]

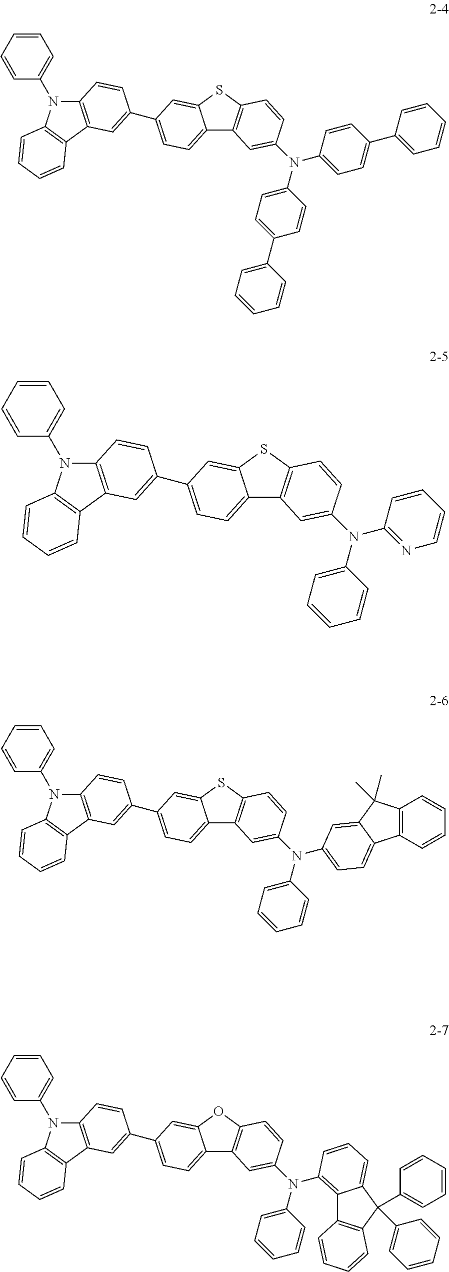 [SMILES: c1ccc(-c2ccc(N(c3ccc(-c4ccccc4)cc3)c3ccc4sc5cc(-c6ccc7c(c6)c6ccccc6n7-c6ccccc6)ccc5c4c3)cc2)cc1 ; c1ccc(N(c2ccc3sc4cc(-c5ccc6c(c5)c5ccccc5n6-c5ccccc5)ccc4c3c2)c2ccccn2)cc1 ; CC1(C)c2ccccc2-c2ccc(N(c3ccccc3)c3ccc4sc5cc(-c6ccc7c(c6)c6ccccc6n7-c6ccccc6)ccc5c4c3)cc21 ; c1ccc(N(c2ccc3oc4cc(-c5ccc6c(c5)c5ccccc5n6-c5ccccc5)ccc4c3c2)c2cccc3c2-c2ccccc2C3(c2ccccc2)c2ccccc2)cc1]

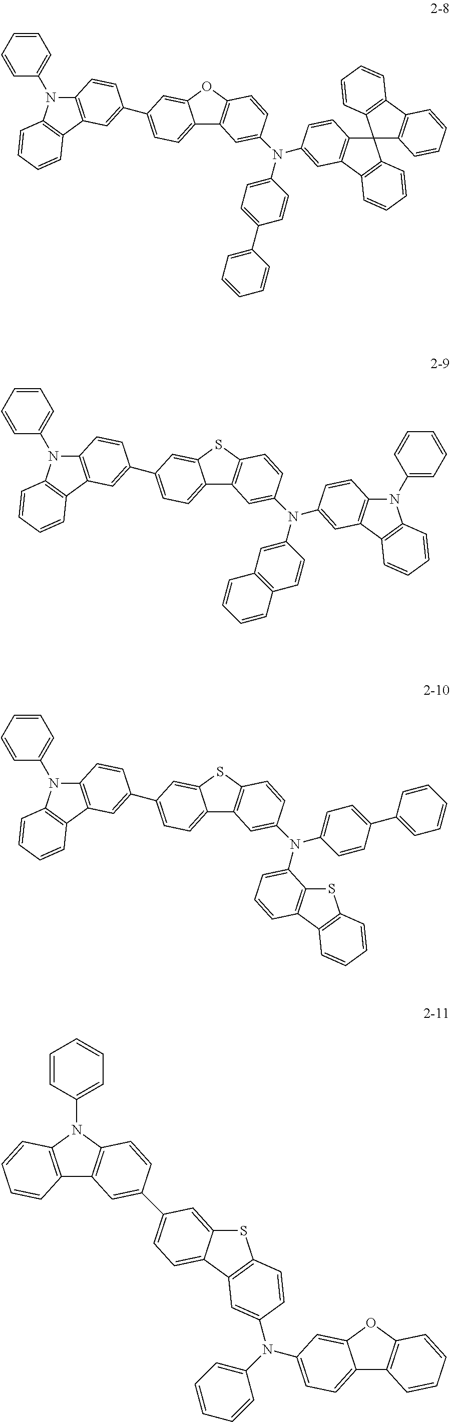 [SMILES: c1ccc(-c2ccc(N(c3ccc4c(c3)-c3ccccc3C43c4ccccc4-c4ccccc43)c3ccc4oc5cc(-c6ccc7c(c6)c6ccccc6n7-c6ccccc6)ccc5c4c3)cc2)cc1 ; c1ccc(-n2c3ccccc3c3cc(-c4ccc5c(c4)sc4ccc(N(c6ccc7ccccc7c6)c6ccc7c(c6)c6ccccc6n7-c6ccccc6)cc45)ccc32)cc1 ; c1ccc(-c2ccc(N(c3ccc4sc5cc(-c6ccc7c(c6)c6ccccc6n7-c6ccccc6)ccc5c4c3)c3cccc4c3sc3ccccc34)cc2)cc1 ; c1ccc(N(c2ccc3c(c2)oc2ccccc23)c2ccc3sc4cc(-c5ccc6c(c5)c5ccccc5n6-c5ccccc5)ccc4c3c2)cc1]

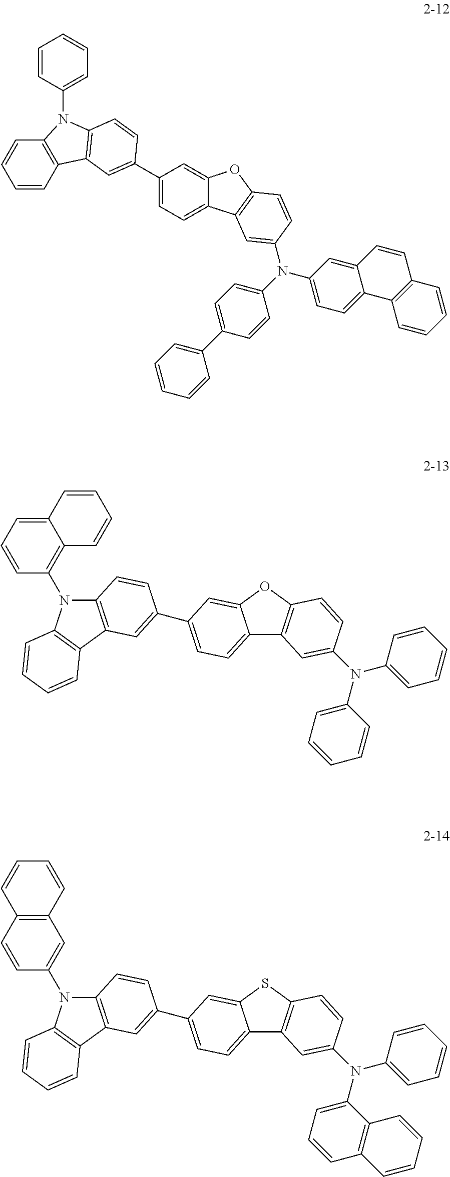 [SMILES: c1ccc(-c2ccc(N(c3ccc4c(ccc5ccccc54)c3)c3ccc4oc5cc(-c6ccc7c(c6)c6ccccc6n7-c6ccccc6)ccc5c4c3)cc2)cc1 ; c1ccc(N(c2ccccc2)c2ccc3oc4cc(-c5ccc6c(c5)c5ccccc5n6-c5cccc6ccccc56)ccc4c3c2)cc1 ; c1ccc(N(c2ccc3sc4cc(-c5ccc6c(c5)c5ccccc5n6-c5ccc6ccccc6c5)ccc4c3c2)c2cccc3ccccc23)cc1]

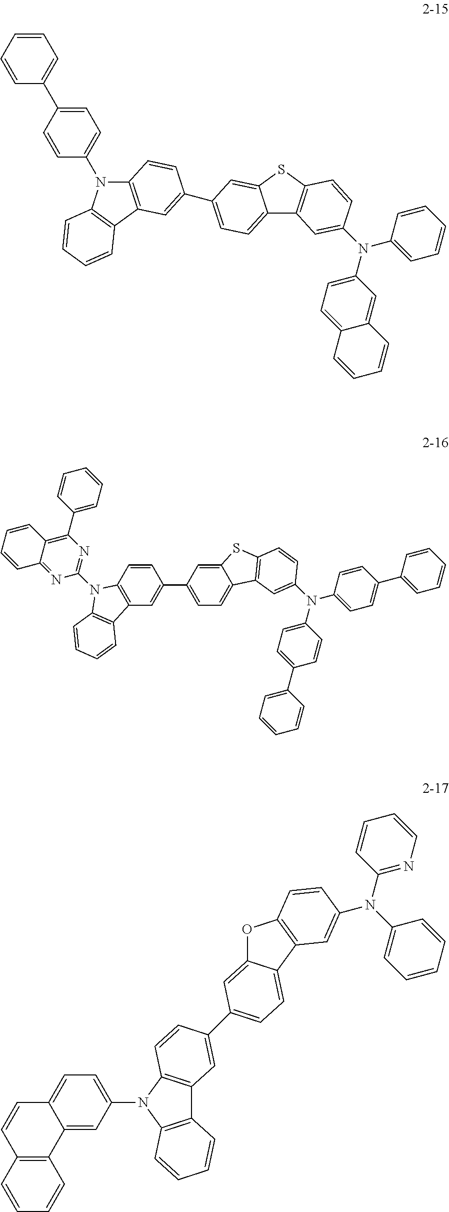 [SMILES: c1ccc(-c2ccc(-n3c4ccccc4c4cc(-c5ccc6c(c5)sc5ccc(N(c7ccccc7)c7ccc8ccccc8c7)cc56)ccc43)cc2)cc1 ; c1ccc(-c2ccc(N(c3ccc(-c4ccccc4)cc3)c3ccc4sc5cc(-c6ccc7c(c6)c6ccccc6n7-c6nc(-c7ccccc7)c7ccccc7n6)ccc5c4c3)cc2)cc1 ; c1ccc(N(c2ccc3oc4cc(-c5ccc6c(c5)c5ccccc5n6-c5ccc6ccc7ccccc7c6c5)ccc4c3c2)c2ccccn2)cc1]

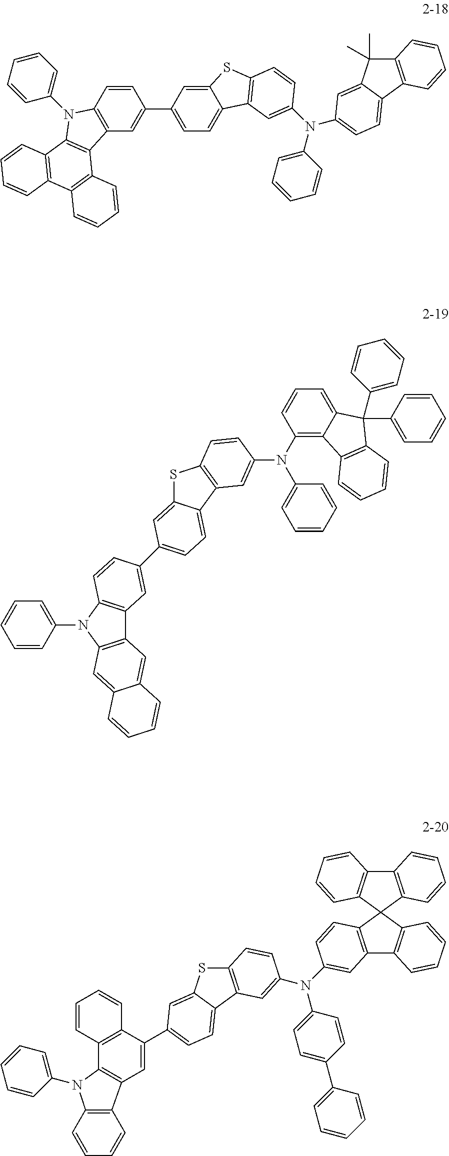 [SMILES: CC1(C)c2ccccc2-c2ccc(N(c3ccccc3)c3ccc4sc5cc(-c6ccc7c(c6)c6c8ccccc8c8ccccc8c6n7-c6ccccc6)ccc5c4c3)cc21 ; c1ccc(N(c2ccc3sc4cc(-c5ccc6c(c5)c5cc7ccccc7cc5n6-c5ccccc5)ccc4c3c2)c2cccc3c2-c2ccccc2C3(c2ccccc2)c2ccccc2)cc1 ; c1ccc(-c2ccc(N(c3ccc4c(c3)-c3ccccc3C43c4ccccc4-c4ccccc43)c3ccc4sc5cc(-c6cc7c8ccccc8n(-c8ccccc8)c7c7ccccc67)ccc5c4c3)cc2)cc1]

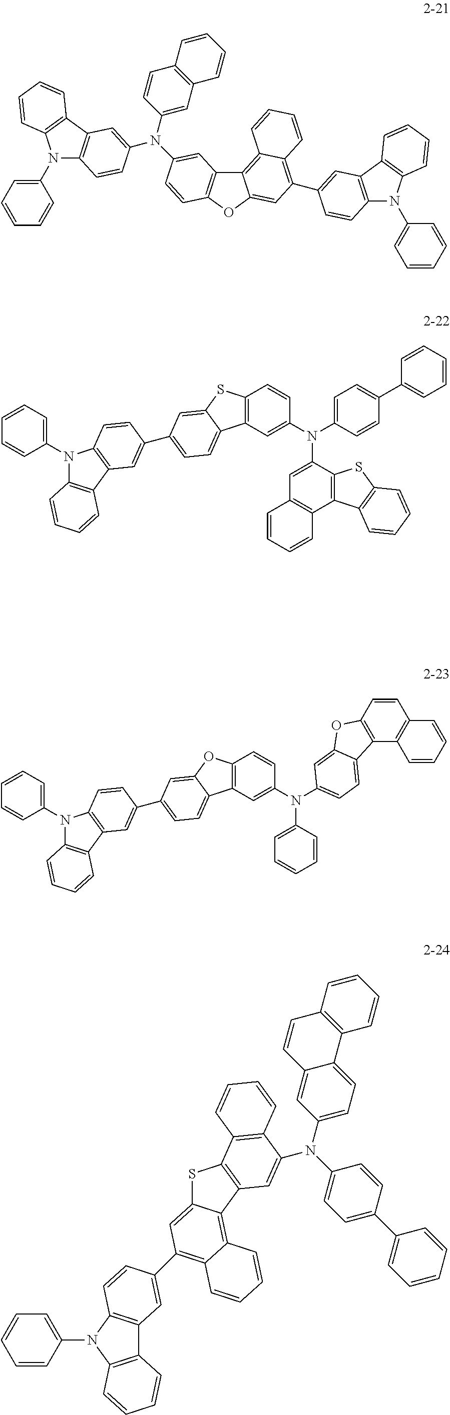 [SMILES: c1ccc(-n2c3ccccc3c3cc(-c4cc5oc6ccc(N(c7ccc8ccccc8c7)c7ccc8c(c7)c7ccccc7n8-c7ccccc7)cc6c5c5ccccc45)ccc32)cc1 ; c1ccc(-c2ccc(N(c3ccc4sc5cc(-c6ccc7c(c6)c6ccccc6n7-c6ccccc6)ccc5c4c3)c3cc4ccccc4c4c3sc3ccccc34)cc2)cc1 ; c1ccc(N(c2ccc3c(c2)oc2ccc4ccccc4c23)c2ccc3oc4cc(-c5ccc6c(c5)c5ccccc5n6-c5ccccc5)ccc4c3c2)cc1 ; c1ccc(-c2ccc(N(c3ccc4c(ccc5ccccc54)c3)c3cc4c(sc5cc(-c6ccc7c(c6)c6ccccc6n7-c6ccccc6)c6ccccc6c54)c4ccccc34)cc2)cc1]

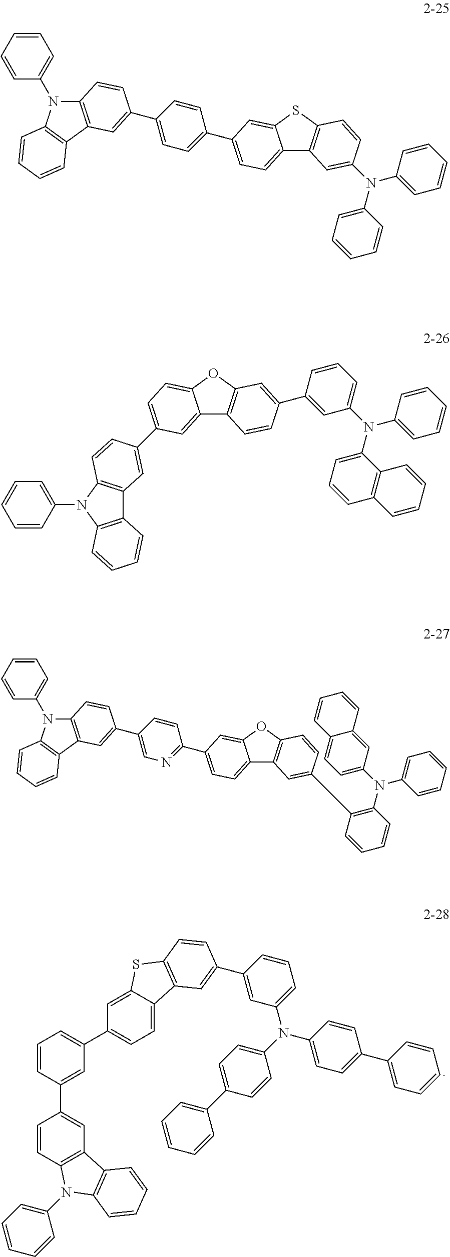 [SMILES: c1ccc(N(c2ccccc2)c2ccc3sc4cc(-c5ccc(-c6ccc7c(c6)c6ccccc6n7-c6ccccc6)cc5)ccc4c3c2)cc1 ; c1ccc(N(c2cccc(-c3ccc4c(c3)oc3ccc(-c5ccc6c(c5)c5ccccc5n6-c5ccccc5)cc34)c2)c2cccc3ccccc23)cc1 ; c1ccc(N(c2ccc3ccccc3c2)c2ccccc2-c2ccc3oc4cc(-c5ccc(-c6ccc7c(c6)c6ccccc6n7-c6ccccc6)cn5)ccc4c3c2)cc1 ; c1ccc(-c2ccc(N(c3ccc(-c4ccccc4)cc3)c3cccc(-c4ccc5sc6cc(-c7cccc(-c8ccc9c(c8)c8ccccc8n9-c8ccccc8)c7)ccc6c5c4)c3)cc2)cc1]

4. An organic electric element comprising a first electrode; a second electrode; and an organic material layer formed between the first electrode and the second electrode; wherein the organic material layer comprises an hole injection layer, an hole transport layer, an emitting auxiliary layer and an emitting layer, wherein the organic material layer comprises the compound of claim 1.

5. The organic electric element according to claim 4, wherein the organic electric element comprises the compound as a single compound or a mixture of two or more of the compounds having different structures in at least one of the hole injection layer, the hole transport layer, the emitting auxiliary layer and the emitting layer.

6. The organic electric element according to claim 5, wherein the hole transport layer or the emitting auxiliary layer comprises the compound alonc as a single compound or a mixture of two or more of the compounds having different structures.

7. The organic electric element according to claim 4, wherein the emitting auxiliary layer comprises the compound, and the hole transport layer comprises a compound represented by Formula (5): 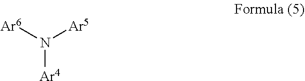
wherein,
1) Ar⁴ and Ar⁵ are each independently selected from the group consisting of a C₆-C₆₀ aryl group; a fluorenyl group; a C₂-C₆₀ heterocyclic group including at least one hetero atom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; and -L′-N(Rₐ)(R_{b})(where, L′ may be selected from the group consisting of a single bond; a C₆-C₆₀ arylene group; a fluorenylene group; a fused ring group of a C₃-C₆₀ aliphatic ring and a C₆-C₆₀ aromatic ring; and a C₂-C₆₀ heterocyclic, and the Rₐ and R_{b} may be independently selected from the group consisting of a C₆-C₆₀ aryl group; a fluorenyl group; a fused ring group of a C₃-C₆₀ aliphatic ring and a C₆-C₆₀ aromatic ring; and a C₂-C₆₀ heterocyclic group containing at least one hetero atom of O, N, S, Si, or P), or Ar⁴ and Ar⁵ may be bonded to each other to form a ring,
2) Ar⁶ is selected from any one of the following Formulas (5-a), (5-b) and (5-c): 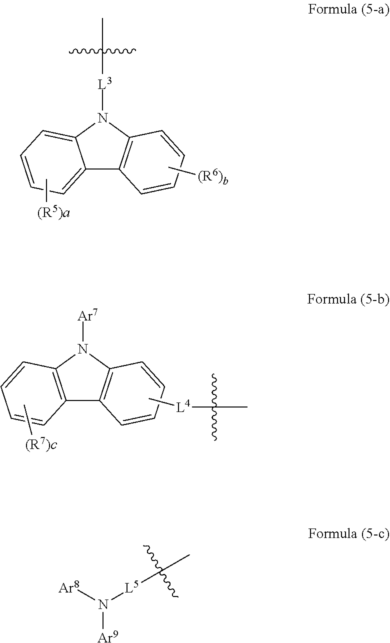
3) a, b, and c are integers of 0 to 4, and R⁵, R⁶ and R⁷ are the same or different and are each independently selected from a deuterium; a halogen; the group consisting of a C₆-C₆₀ aryl group; a fluorenyl group; a C₂-C₆₀ heterocyclic group including at least one hetero atom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; and -L′-N(Rₐ)(R_{b}); or in case a, b and c are 2 or more, and R⁵, R⁶ and R⁷ are each in plural and are the same or different, or a plurality of R⁵ or a plurality of R⁶ or a plurality of R⁷ may be bonded to each other to form a ring,
4) L³ and L⁵ are each independently selected from the group consisting of a C₆-C₆₀ aryl group; a fluorenylene group; a fused ring group of a C₃-C₆₀ aliphatic ring and a C₆-C₆₀ aromatic ring; and a C₂-C₆₀ heterocyclic group including at least one hetero atom of O, N, S, Si or P;
5) L⁴ is selected from a single bond of C₆-C₆₀; an arylene group; a fluorenylene group; a fused ring group of a C₃-C₆₀ aliphatic ring and a C₆-C₆₀ aromatic ring; and a C₂-C₆₀ heterocyclic group including at least one hetero atom of O, N, S, Si or P;
6) Ar⁷, Ar⁸ and Ar⁹ are each independently selected from the group consisting of a C₆-C₆₀ aryl group; a fluorenyl group; a C₂-C₆₀ heterocyclic group including at least one hetero atom of O, N, S, Si or P; a fused ring group of a C₃-C₆₀ aliphatic ring and a C₆-C₆₀ aromatic ring;
wherein the aryl group, the fluorenyl group, the arylene group, the heterocyclic group, the fused ring group, the alkyl group, the alkenyl group, the alkoxy group and the aryloxy group may be each substituted with one or more substituents selected from the group consisting of a deuterium; halogen; a silane group substituted or unsubstituted with a C₁-C₂₀ alkyl group ora C₆-C₆₀ aryl group; a siloxane group; a boron group; a germanium group; a cyano; a nitro group; -L′-N(Rₐ)(R_{b}); a C₁-C₂₀ alkylthio group; C₁-C₂₀ alkoxyl group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂-C₂₀ heterocyclic group; C₃-C₂₀ cycloalkyl group; C₇-C₂₀ arylalkyl group and C₈-C₂₀ arylalkenyl group, and also, these substituents may be bonded to each other to form a ring, wherein the term ‘ring’ means C₃-C₆₀ aliphatic ring or C₆-C₆₀ aromatic ring or a C₂-C₆₀ heterocyclic ring or a fused ring formed by the combination of them, and includes a saturated or unsaturated ring.

8. The organic electric element according to claim 7,
wherein the compound represented by Formula (5) is any one of compounds represented by the following Formulas (5-1) to (5-71):

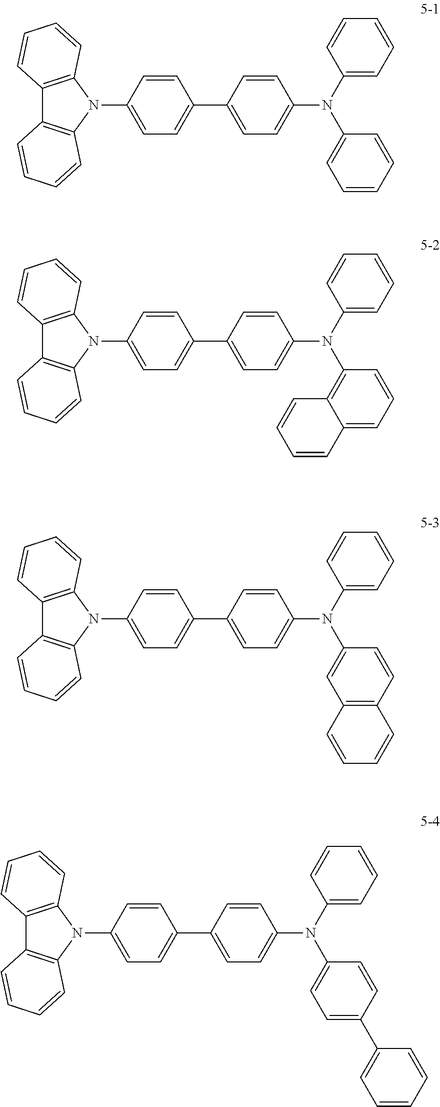 [SMILES: c1ccc(N(c2ccccc2)c2ccc(-c3ccc(-n4c5ccccc5c5ccccc54)cc3)cc2)cc1 ; c1ccc(N(c2ccc(-c3ccc(-n4c5ccccc5c5ccccc54)cc3)cc2)c2cccc3ccccc23)cc1 ; c1ccc(N(c2ccc(-c3ccc(-n4c5ccccc5c5ccccc54)cc3)cc2)c2ccc3ccccc3c2)cc1 ; c1ccc(-c2ccc(N(c3ccccc3)c3ccc(-c4ccc(-n5c6ccccc6c6ccccc65)cc4)cc3)cc2)cc1]

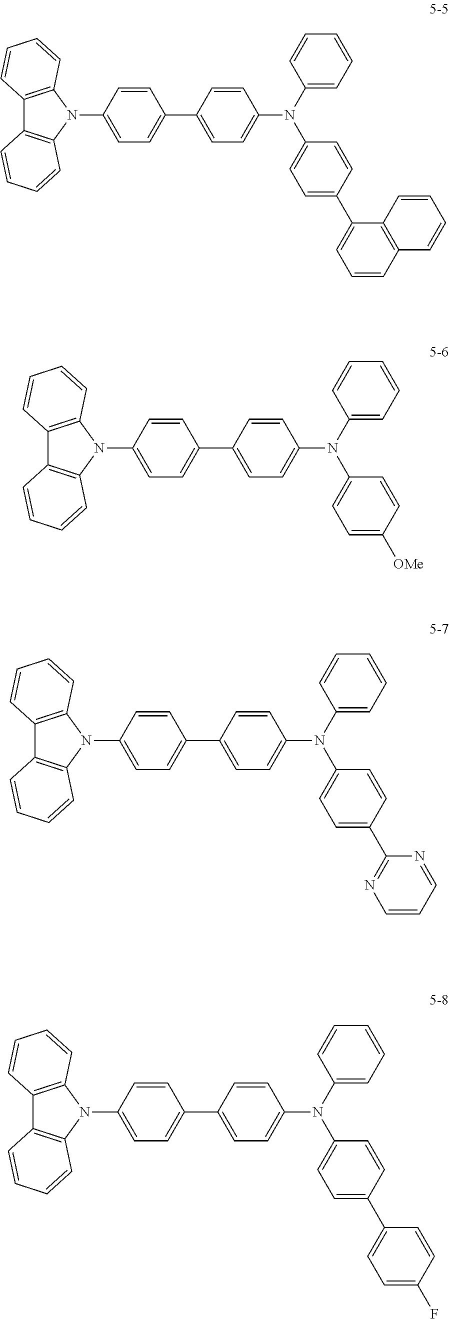 [SMILES: c1ccc(N(c2ccc(-c3ccc(-n4c5ccccc5c5ccccc54)cc3)cc2)c2ccc(-c3cccc4ccccc34)cc2)cc1 ; COc1ccc(N(c2ccccc2)c2ccc(-c3ccc(-n4c5ccccc5c5ccccc54)cc3)cc2)cc1 ; c1ccc(N(c2ccc(-c3ccc(-n4c5ccccc5c5ccccc54)cc3)cc2)c2ccc(-c3ncccn3)cc2)cc1 ; Fc1ccc(-c2ccc(N(c3ccccc3)c3ccc(-c4ccc(-n5c6ccccc6c6ccccc65)cc4)cc3)cc2)cc1]

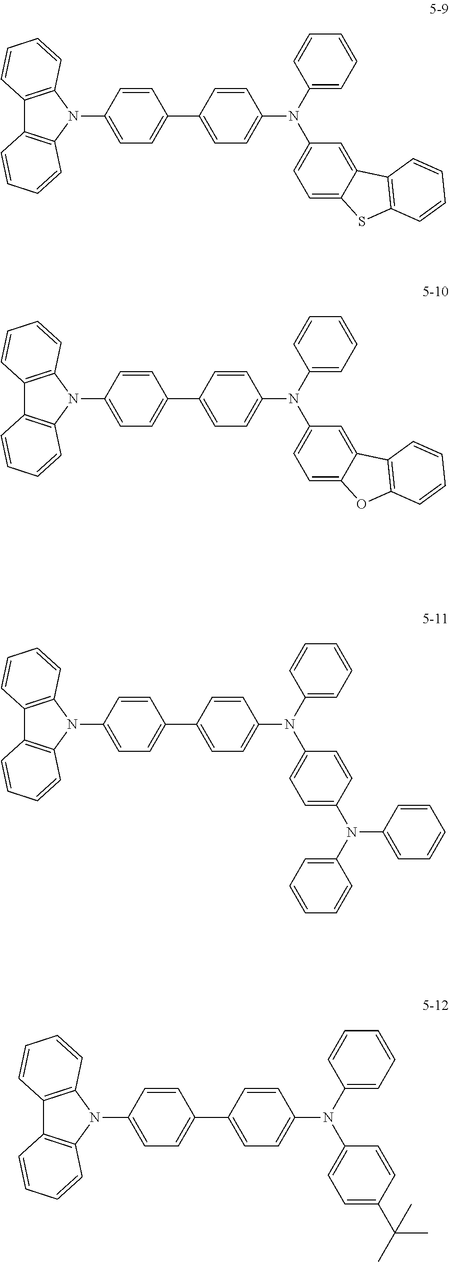 [SMILES: c1ccc(N(c2ccc(-c3ccc(-n4c5ccccc5c5ccccc54)cc3)cc2)c2ccc3sc4ccccc4c3c2)cc1 ; c1ccc(N(c2ccc(-c3ccc(-n4c5ccccc5c5ccccc54)cc3)cc2)c2ccc3oc4ccccc4c3c2)cc1 ; c1ccc(N(c2ccccc2)c2ccc(N(c3ccccc3)c3ccc(-c4ccc(-n5c6ccccc6c6ccccc65)cc4)cc3)cc2)cc1 ; CC(C)(C)c1ccc(N(c2ccccc2)c2ccc(-c3ccc(-n4c5ccccc5c5ccccc54)cc3)cc2)cc1]

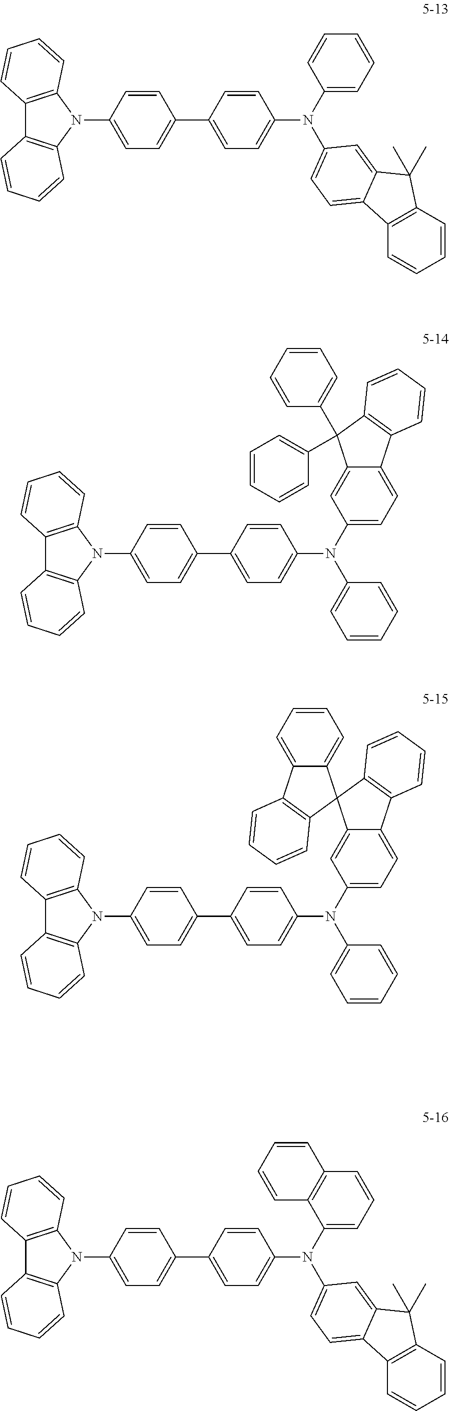 [SMILES: CC1(C)c2ccccc2-c2ccc(N(c3ccccc3)c3ccc(-c4ccc(-n5c6ccccc6c6ccccc65)cc4)cc3)cc21 ; c1ccc(N(c2ccc(-c3ccc(-n4c5ccccc5c5ccccc54)cc3)cc2)c2ccc3c(c2)C(c2ccccc2)(c2ccccc2)c2ccccc2-3)cc1 ; c1ccc(N(c2ccc(-c3ccc(-n4c5ccccc5c5ccccc54)cc3)cc2)c2ccc3c(c2)C2(c4ccccc4-c4ccccc42)c2ccccc2-3)cc1 ; CC1(C)c2ccccc2-c2ccc(N(c3ccc(-c4ccc(-n5c6ccccc6c6ccccc65)cc4)cc3)c3cccc4ccccc34)cc21]

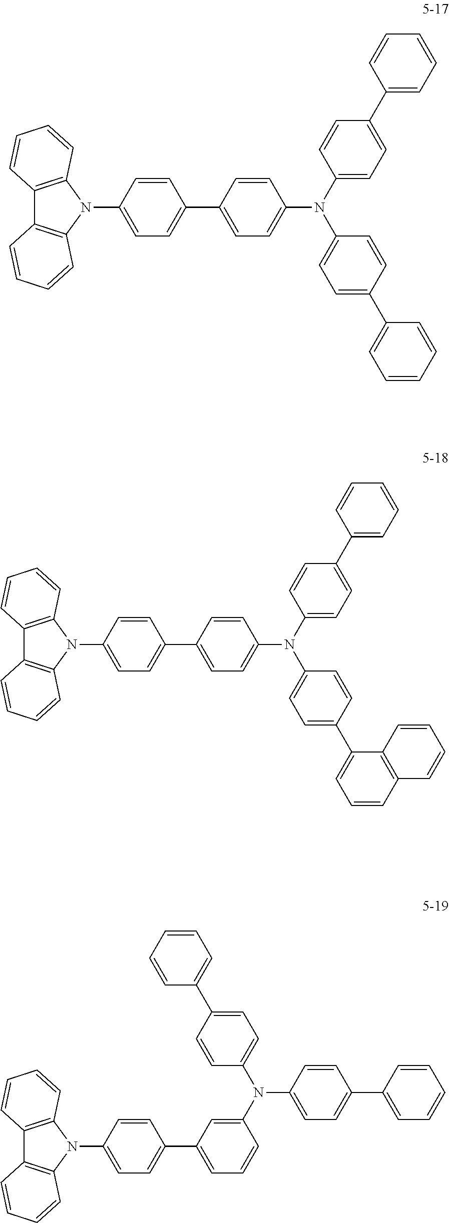 [SMILES: c1ccc(-c2ccc(N(c3ccc(-c4ccccc4)cc3)c3ccc(-c4ccc(-n5c6ccccc6c6ccccc65)cc4)cc3)cc2)cc1 ; c1ccc(-c2ccc(N(c3ccc(-c4ccc(-n5c6ccccc6c6ccccc65)cc4)cc3)c3ccc(-c4cccc5ccccc45)cc3)cc2)cc1 ; c1ccc(-c2ccc(N(c3ccc(-c4ccccc4)cc3)c3cccc(-c4ccc(-n5c6ccccc6c6ccccc65)cc4)c3)cc2)cc1]

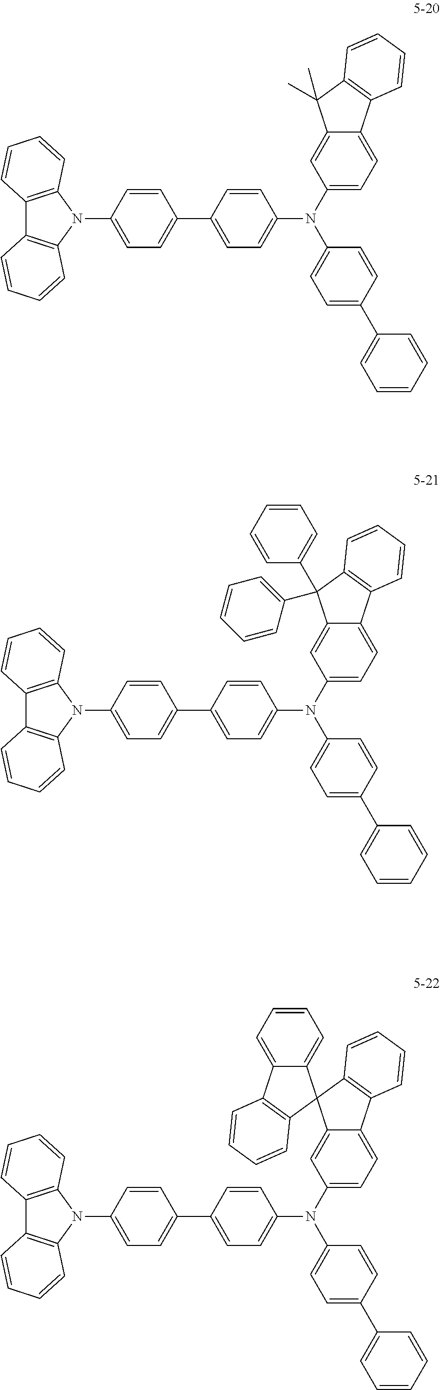 [SMILES: CC1(C)c2ccccc2-c2ccc(N(c3ccc(-c4ccccc4)cc3)c3ccc(-c4ccc(-n5c6ccccc6c6ccccc65)cc4)cc3)cc21 ; c1ccc(-c2ccc(N(c3ccc(-c4ccc(-n5c6ccccc6c6ccccc65)cc4)cc3)c3ccc4c(c3)C(c3ccccc3)(c3ccccc3)c3ccccc3-4)cc2)cc1 ; c1ccc(-c2ccc(N(c3ccc(-c4ccc(-n5c6ccccc6c6ccccc65)cc4)cc3)c3ccc4c(c3)C3(c5ccccc5-c5ccccc53)c3ccccc3-4)cc2)cc1]

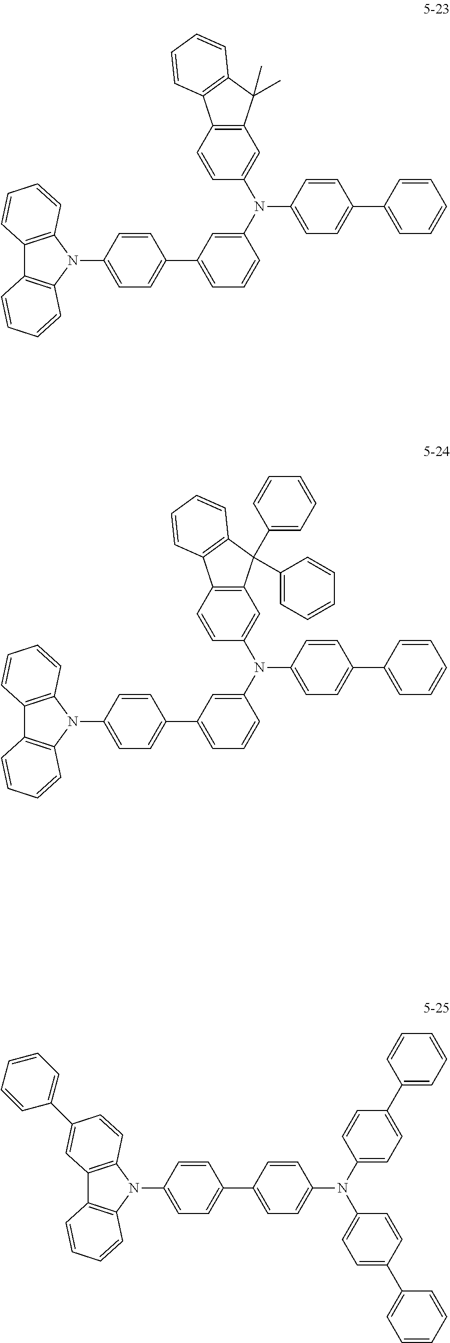 [SMILES: CC1(C)c2ccccc2-c2ccc(N(c3ccc(-c4ccccc4)cc3)c3cccc(-c4ccc(-n5c6ccccc6c6ccccc65)cc4)c3)cc21 ; c1ccc(-c2ccc(N(c3cccc(-c4ccc(-n5c6ccccc6c6ccccc65)cc4)c3)c3ccc4c(c3)C(c3ccccc3)(c3ccccc3)c3ccccc3-4)cc2)cc1 ; c1ccc(-c2ccc(N(c3ccc(-c4ccccc4)cc3)c3ccc(-c4ccc(-n5c6ccccc6c6cc(-c7ccccc7)ccc65)cc4)cc3)cc2)cc1]

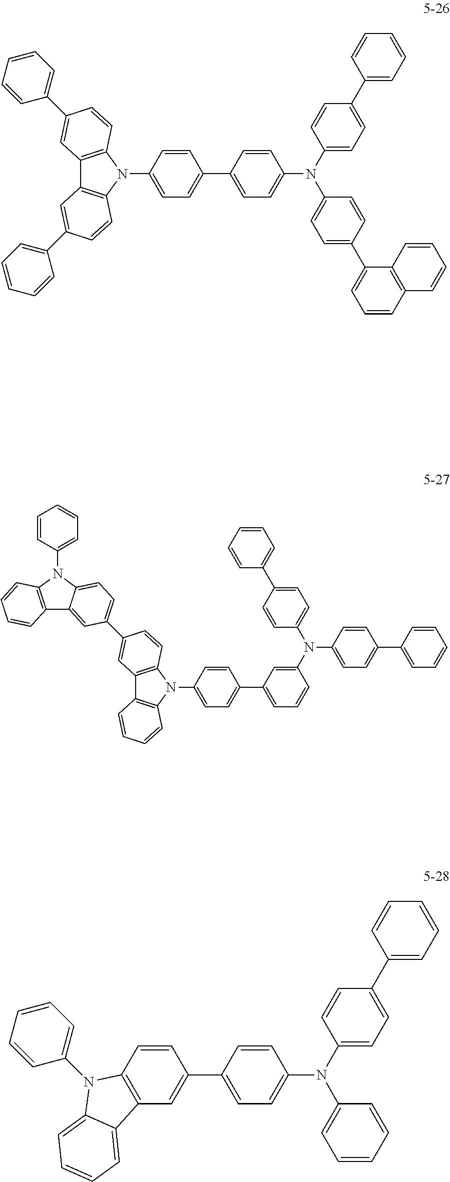 [SMILES: c1ccc(-c2ccc(N(c3ccc(-c4ccc(-n5c6ccc(-c7ccccc7)cc6c6cc(-c7ccccc7)ccc65)cc4)cc3)c3ccc(-c4cccc5ccccc45)cc3)cc2)cc1 ; c1ccc(-c2ccc(N(c3ccc(-c4ccccc4)cc3)c3cccc(-c4ccc(-n5c6ccccc6c6cc(-c7ccc8c(c7)c7ccccc7n8-c7ccccc7)ccc65)cc4)c3)cc2)cc1 ; c1ccc(-c2ccc(N(c3ccccc3)c3ccc(-c4ccc5c(c4)c4ccccc4n5-c4ccccc4)cc3)cc2)cc1]

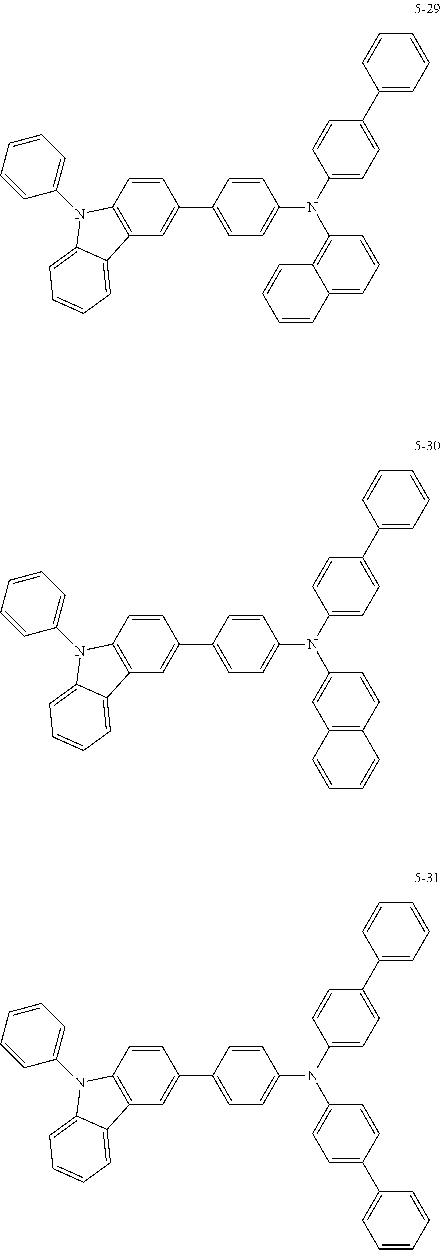 [SMILES: c1ccc(-c2ccc(N(c3ccc(-c4ccc5c(c4)c4ccccc4n5-c4ccccc4)cc3)c3cccc4ccccc34)cc2)cc1 ; c1ccc(-c2ccc(N(c3ccc(-c4ccc5c(c4)c4ccccc4n5-c4ccccc4)cc3)c3ccc4ccccc4c3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1]

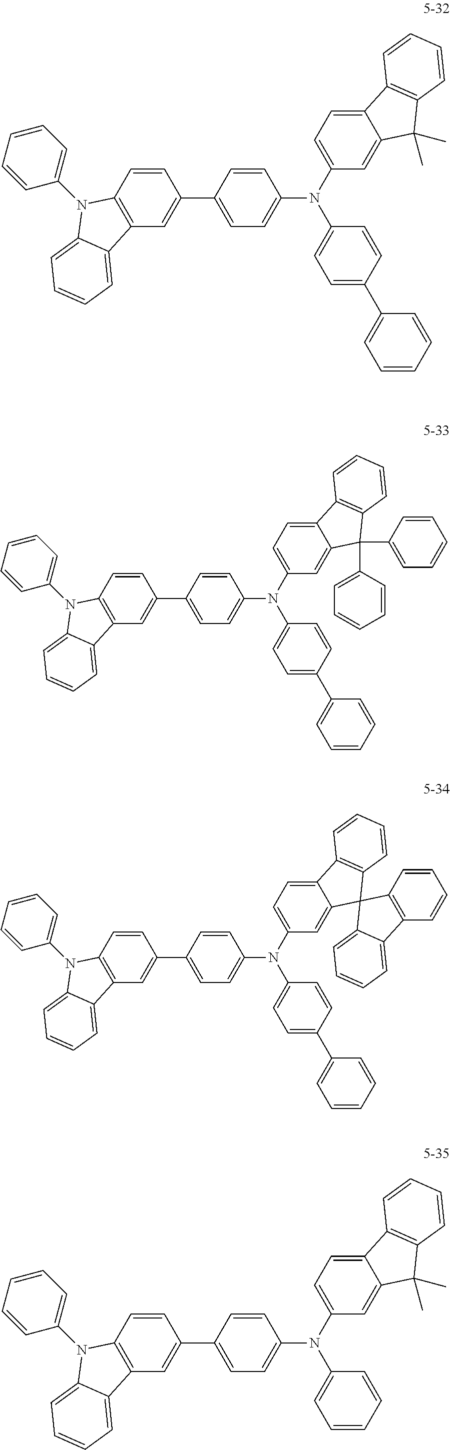 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1 ; CC1(C)c2ccccc2-c2ccc(N(c3ccccc3)c3ccc(-c4ccc5c(c4)c4ccccc4n5-c4ccccc4)cc3)cc21]

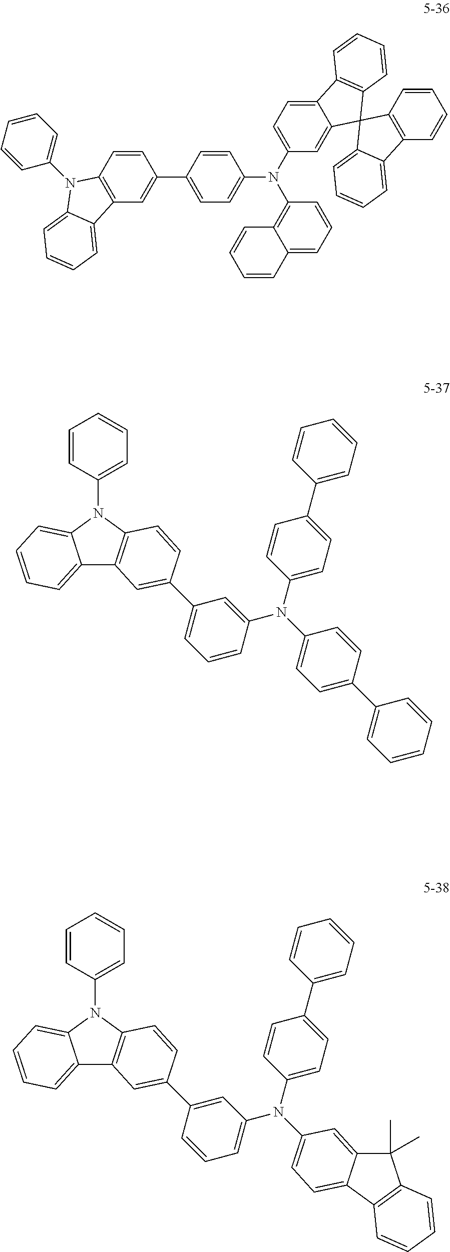 [SMILES: c1ccc(-n2c3ccccc3c3cc(-c4ccc(N(c5ccc6c(c5)C5(c7ccccc7-c7ccccc75)c5ccccc5-6)c5cccc6ccccc56)cc4)ccc32)cc1 ; c1ccc(-c2ccc(N(c3ccc(-c4ccccc4)cc3)c3cccc(-c4ccc5c(c4)c4ccccc4n5-c4ccccc4)c3)cc2)cc1 ; CC1(C)c2ccccc2-c2ccc(N(c3ccc(-c4ccccc4)cc3)c3cccc(-c4ccc5c(c4)c4ccccc4n5-c4ccccc4)c3)cc21]

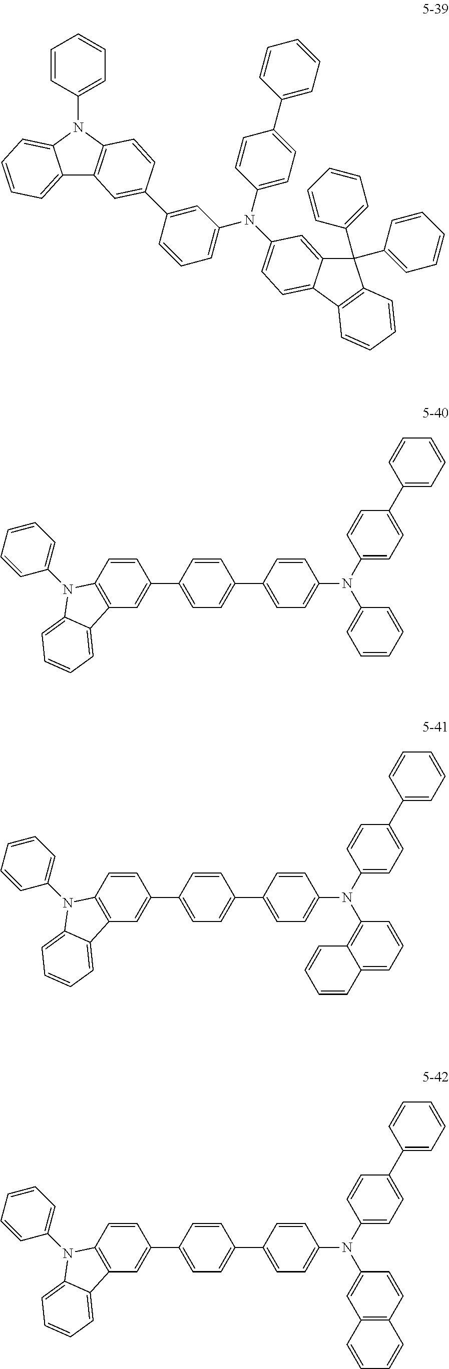 [SMILES: c1ccc(-c2ccc(N(c3cccc(-c4ccc5c(c4)c4ccccc4n5-c4ccccc4)c3)c3ccc4c(c3)C(c3ccccc3)(c3ccccc3)c3ccccc3-4)cc2)cc1 ; c1ccc(-c2ccc(N(c3ccccc3)c3ccc(-c4ccc(-c5ccc6c(c5)c5ccccc5n6-c5ccccc5)cc4)cc3)cc2)cc1 ; c1ccc(-c2ccc(N(c3ccc(-c4ccc(-c5ccc6c(c5)c5ccccc5n6-c5ccccc5)cc4)cc3)c3cccc4ccccc34)cc2)cc1 ; c1ccc(-c2ccc(N(c3ccc(-c4ccc(-c5ccc6c(c5)c5ccccc5n6-c5ccccc5)cc4)cc3)c3ccc4ccccc4c3)cc2)cc1]

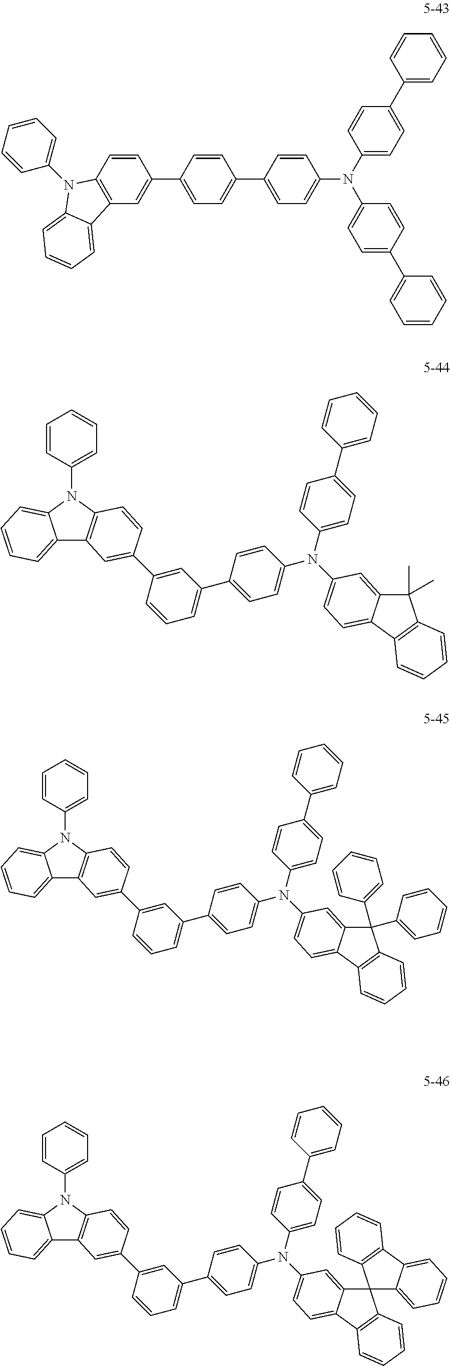 [SMILES: c1ccc(-c2ccc(N(c3ccc(-c4ccccc4)cc3)c3ccc(-c4ccc(-c5ccc6c(c5)c5ccccc5n6-c5ccccc5)cc4)cc3)cc2)cc1 ; CC1(C)c2ccccc2-c2ccc(N(c3ccc(-c4ccccc4)cc3)c3ccc(-c4cccc(-c5ccc6c(c5)c5ccccc5n6-c5ccccc5)c4)cc3)cc21 ; c1ccc(-c2ccc(N(c3ccc(-c4cccc(-c5ccc6c(c5)c5ccccc5n6-c5ccccc5)c4)cc3)c3ccc4c(c3)C(c3ccccc3)(c3ccccc3)c3ccccc3-4)cc2)cc1 ; c1ccc(-c2ccc(N(c3ccc(-c4cccc(-c5ccc6c(c5)c5ccccc5n6-c5ccccc5)c4)cc3)c3ccc4c(c3)C3(c5ccccc5-c5ccccc53)c3ccccc3-4)cc2)cc1]

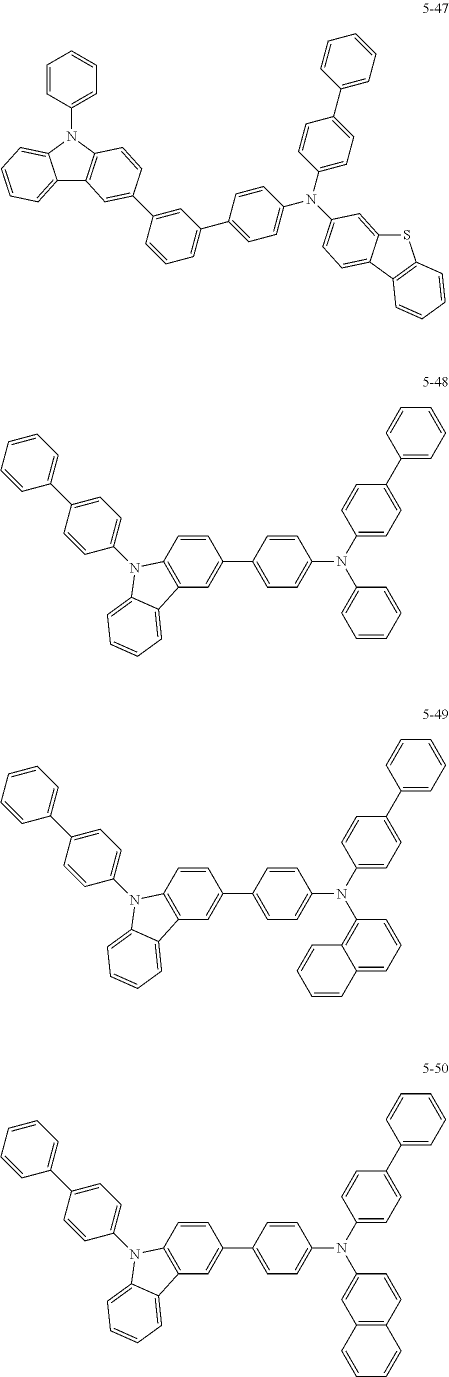 [SMILES: c1ccc(-c2ccc(N(c3ccc(-c4cccc(-c5ccc6c(c5)c5ccccc5n6-c5ccccc5)c4)cc3)c3ccc4c(c3)sc3ccccc34)cc2)cc1 ; c1ccc(-c2ccc(N(c3ccccc3)c3ccc(-c4ccc5c(c4)c4ccccc4n5-c4ccc(-c5ccccc5)cc4)cc3)cc2)cc1 ; c1ccc(-c2ccc(N(c3ccc(-c4ccc5c(c4)c4ccccc4n5-c4ccc(-c5ccccc5)cc4)cc3)c3cccc4ccccc34)cc2)cc1 ; c1ccc(-c2ccc(N(c3ccc(-c4ccc5c(c4)c4ccccc4n5-c4ccc(-c5ccccc5)cc4)cc3)c3ccc4ccccc4c3)cc2)cc1]

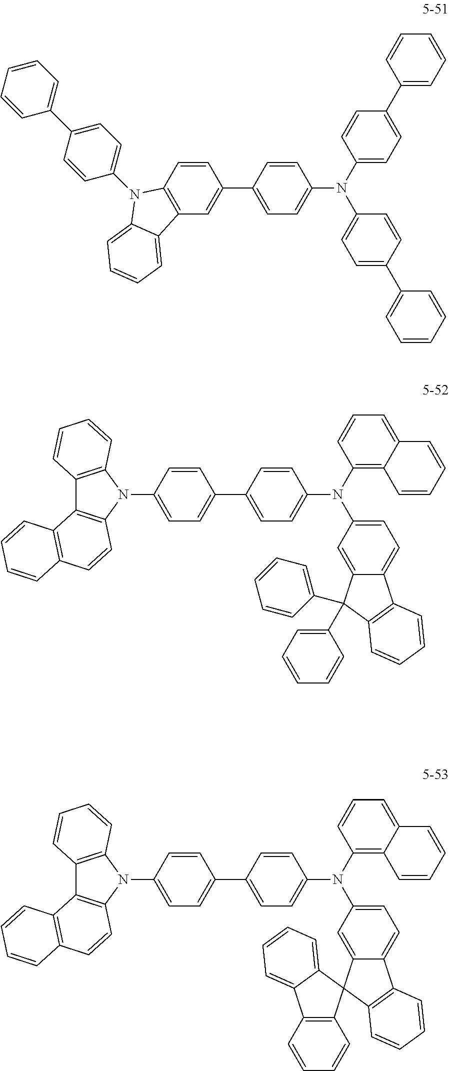 [SMILES: c1ccc(-c2ccc(N(c3ccc(-c4ccccc4)cc3)c3ccc(-c4ccc5c(c4)c4ccccc4n5-c4ccc(-c5ccccc5)cc4)cc3)cc2)cc1 ; c1ccc(C2(c3ccccc3)c3ccccc3-c3ccc(N(c4ccc(-c5ccc(-n6c7ccccc7c7c8ccccc8ccc76)cc5)cc4)c4cccc5ccccc45)cc32)cc1 ; c1ccc2c(c1)-c1ccccc1C21c2ccccc2-c2ccc(N(c3ccc(-c4ccc(-n5c6ccccc6c6c7ccccc7ccc65)cc4)cc3)c3cccc4ccccc34)cc21]

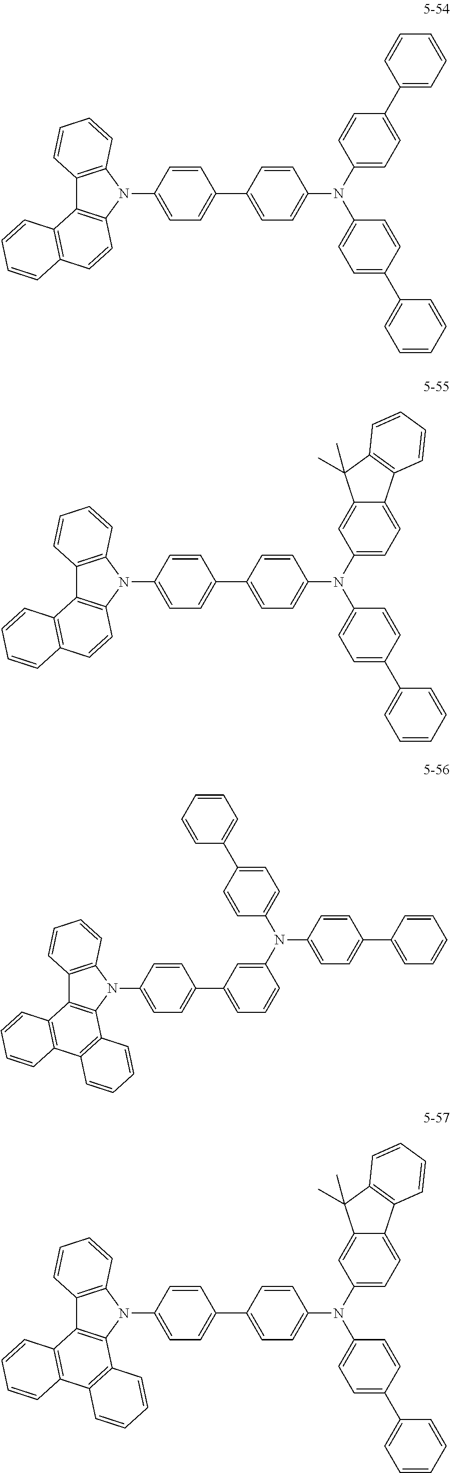 [SMILES: c1ccc(-c2ccc(N(c3ccc(-c4ccccc4)cc3)c3ccc(-c4ccc(-n5c6ccccc6c6c7ccccc7ccc65)cc4)cc3)cc2)cc1 ; CC1(C)c2ccccc2-c2ccc(N(c3ccc(-c4ccccc4)cc3)c3ccc(-c4ccc(-n5c6ccccc6c6c7ccccc7ccc65)cc4)cc3)cc21 ; c1ccc(-c2ccc(N(c3ccc(-c4ccccc4)cc3)c3cccc(-c4ccc(-n5c6ccccc6c6c7ccccc7c7ccccc7c65)cc4)c3)cc2)cc1 ; CC1(C)c2ccccc2-c2ccc(N(c3ccc(-c4ccccc4)cc3)c3ccc(-c4ccc(-n5c6ccccc6c6c7ccccc7c7ccccc7c65)cc4)cc3)cc21]

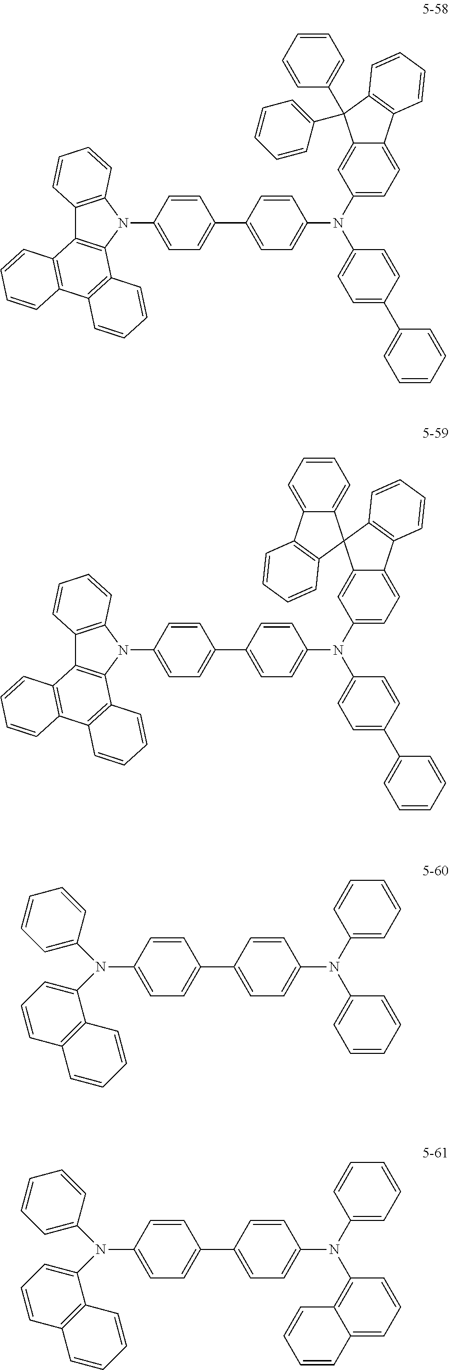 [SMILES: c1ccc(-c2ccc(N(c3ccc(-c4ccc(-n5c6ccccc6c6c7ccccc7c7ccccc7c65)cc4)cc3)c3ccc4c(c3)C(c3ccccc3)(c3ccccc3)c3ccccc3-4)cc2)cc1 ; c1ccc(-c2ccc(N(c3ccc(-c4ccc(-n5c6ccccc6c6c7ccccc7c7ccccc7c65)cc4)cc3)c3ccc4c(c3)C3(c5ccccc5-c5ccccc53)c3ccccc3-4)cc2)cc1 ; c1ccc(N(c2ccccc2)c2ccc(-c3ccc(N(c4ccccc4)c4cccc5ccccc45)cc3)cc2)cc1 ; c1ccc(N(c2ccc(-c3ccc(N(c4ccccc4)c4cccc5ccccc45)cc3)cc2)c2cccc3ccccc23)cc1]

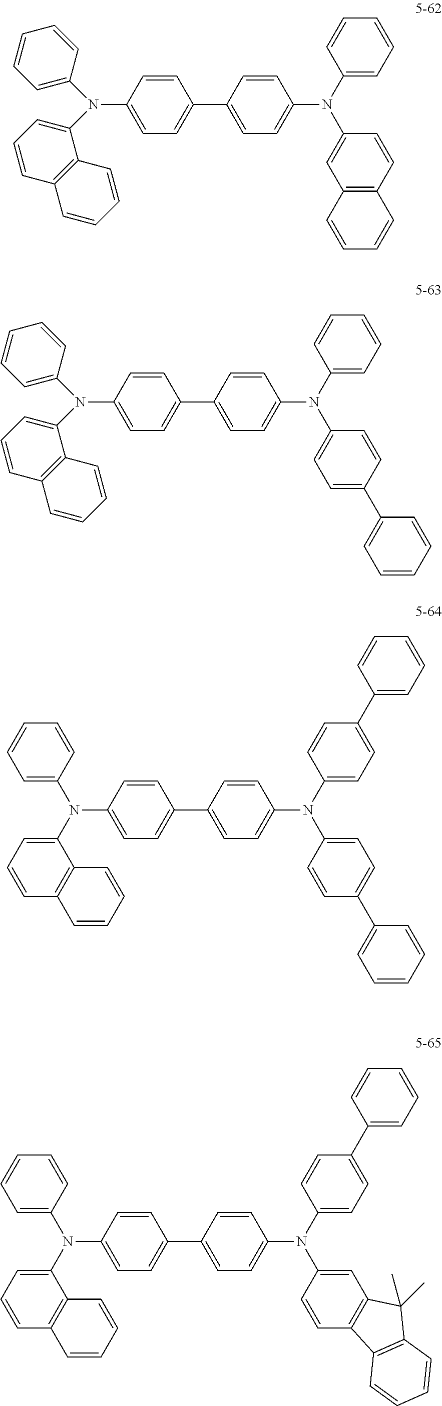 [SMILES: c1ccc(N(c2ccc(-c3ccc(N(c4ccccc4)c4cccc5ccccc45)cc3)cc2)c2ccc3ccccc3c2)cc1 ; c1ccc(-c2ccc(N(c3ccccc3)c3ccc(-c4ccc(N(c5ccccc5)c5cccc6ccccc56)cc4)cc3)cc2)cc1 ; c1ccc(-c2ccc(N(c3ccc(-c4ccccc4)cc3)c3ccc(-c4ccc(N(c5ccccc5)c5cccc6ccccc56)cc4)cc3)cc2)cc1 ; CC1(C)c2ccccc2-c2ccc(N(c3ccc(-c4ccccc4)cc3)c3ccc(-c4ccc(N(c5ccccc5)c5cccc6ccccc56)cc4)cc3)cc21]

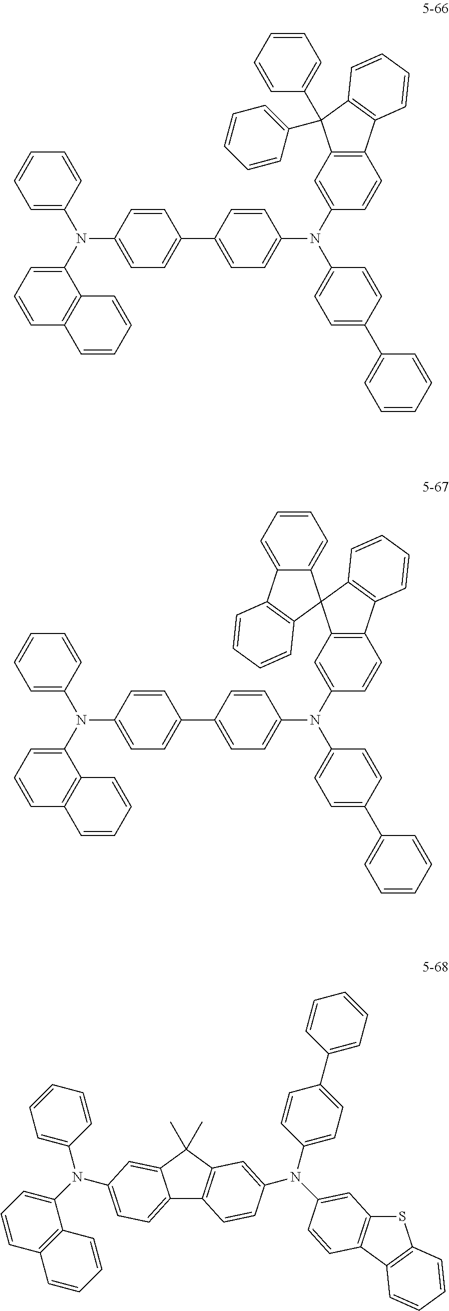 [SMILES: c1ccc(-c2ccc(N(c3ccc(-c4ccc(N(c5ccccc5)c5cccc6ccccc56)cc4)cc3)c3ccc4c(c3)C(c3ccccc3)(c3ccccc3)c3ccccc3-4)cc2)cc1 ; c1ccc(-c2ccc(N(c3ccc(-c4ccc(N(c5ccccc5)c5cccc6ccccc56)cc4)cc3)c3ccc4c(c3)C3(c5ccccc5-c5ccccc53)c3ccccc3-4)cc2)cc1 ; CC1(C)c2cc(N(c3ccc(-c4ccccc4)cc3)c3ccc4c(c3)sc3ccccc34)ccc2-c2ccc(N(c3ccccc3)c3cccc4ccccc34)cc21]

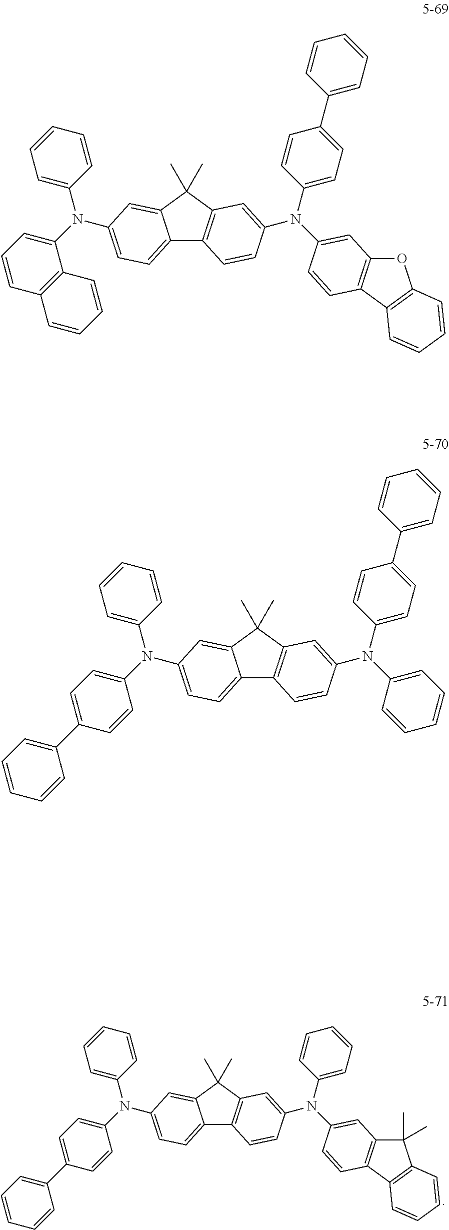 [SMILES: CC1(C)c2cc(N(c3ccc(-c4ccccc4)cc3)c3ccc4c(c3)oc3ccccc34)ccc2-c2ccc(N(c3ccccc3)c3cccc4ccccc34)cc21 ; CC1(C)c2cc(N(c3ccccc3)c3ccc(-c4ccccc4)cc3)ccc2-c2ccc(N(c3ccccc3)c3ccc(-c4ccccc4)cc3)cc21 ; CC1(C)c2ccccc2-c2ccc(N(c3ccccc3)c3ccc4c(c3)C(C)(C)c3cc(N(c5ccccc5)c5ccc(-c6ccccc6)cc5)ccc3-4)cc21]

9. The organic electric element according to claim 7, wherein the emitting auxiliary layer comprises the compound as a single compound or a mixture of two or more of the compounds having different structures.

10. The organic electric element according to claim 4, wherein the organic electric element comprises a light efficiency enhancing layer formed on at least one of the opposite sides to the organic material layer among one side of the first electrode, or one of the opposite sides to the organic material layer among one side of the second electrode.

11. The organic electric element according to claim 4, wherein the organic electric element is formed by one of a spin coating process, a nozzle printing process, an inkjet printing process, a slot coating process, a dip coating process and a roll-to-roll process.

12. An electronic device comprising a display device including the organic electric element according to claim 4; and a control unit for driving the display device.

13. The electronic device according to claim 12, wherein the organic electric element is at least one of an OLED, an organic solar cell, an organic photo conductor, an organic transistor and an element for monochromic or white illumination.
